(19) United States
(12) Patent Application Publication
(10) Pub. No.: US 2023/0340555 A1
(43) Pub. Date: Oct. 26, 2023

(54) PRODUCTION OF GLYCOSYLATED CANNABINOIDS

(71) Applicants: WILLOW BIOSCIENCES, INC., Calagary (CA); EPIMERON USA, INC., Mountain View, CA (US)

(72) Inventors: Mathias Schuetz, Calagary (CA); Gisele Passaia Prietsch, Calagary (CA)

(73) Assignees: WILLOW BIOSCIENCES, INC., Calagary (CA); EPIMERON USA, INC., Mountain View, CA (US)

(21) Appl. No.: 18/311,327

(22) Filed: May 3, 2023

Related U.S. Application Data
(63) Continuation of application No. PCT/US2021/058342, filed on Nov. 5, 2021.
(60) Provisional application No. 63/111,005, filed on Nov. 7, 2020.

(51) Int. Cl. C12N 15/81; C12P 19/46; C12N 9/02; C12N 9/10; C12N 15/52

(52) U.S. Cl. CPC C12P 19/46; C12N 9/0004; C12N 9/1051; C12N 15/52; C12N 15/81; C12Y 121/03007; C12Y 121/03008

(57) ABSTRACT

The present disclosure provides methods for making glycosylated cannabinoids including methods using recombinant host cells comprising a pathway capable of producing a cannabinoid and a heterologous nucleic acid that encodes a UDP-glycosyl transferase. The disclosure also provides compositions of recombinant host cells capable of producing glycosylated cannabinoids, and compositions and uses of the glycosylated cannabinoids.

### CROSS REFERENCE TO RELATED APPLICATIONS

This application is continuation of International Application Number PCT/US2021/058342, filed Nov. 5, 2021, which claims priority of U.S. Provisional Pat. Application No. 63/111,005, filed Nov. 7, 2020, the entirety of each of which is hereby incorporated by reference herein.

### FIELD

The present disclosure provides compositions, methods, and systems related to glycosylated cannabinoids and methods for their preparation.

### REFERENCE TO SEQUENCE LISTING

The official copy of the Sequence Listing is submitted concurrently with the specification via USPTO Patent Center as an WIPO Standard ST26 formatted XML file with file name “13421-005PV1.xml”, a creation date of May 2, 2023, and a size of 149,750 bytes. This Sequence Listing filed via USPTO Patent Center is part of the specification and is incorporated in its entirety by reference herein. This ST26 formatted copy of the Sequence Listing submitted herewith corresponds to the original ST25 formatted Sequence Listing with a file name of “13421-005PV1_SeqList_ST25.txt”, a creation date of Nov. 7, 2020, and a size of 167,669 bytes, that was filed concurrently with U.S. Provisional Pat. Application No. 63/111,005, on Nov. 7, 2020.

### BACKGROUND

The interest of the art in cannabinoids is well established. Thus, for example, the cannabinoid, Δ⁹-tetrahydrocannabinol (Δ⁹-THC) is a psychoactive compound and is therefore used as a recreational drug. Δ⁹-THC can also be employed in the treatment of pain and other medical conditions. Furthermore, it is well known that cannabinoids can be prepared by extraction from plants naturally capable of producing these compounds, such as Cannabis sativa. However, one significant drawback associated with the natural cannabinoid containing plant extracts known to the art is that they contain a variety of chemically similar, but nevertheless distinct, chemical species, which together, in general terms, can be said to constitute the cannabinoid profile of a plant extract. Thus, plant extracts may contain varying relative amounts of Δ⁹-THC, cannabidiol (CBD), and a variety of other cannabinoid compounds. Moreover, and importantly, it is frequently difficult to consistently produce plant extracts comprising chemically identical cannabinoid profiles. In the absence of chemical identity, different cannabinoid preparation batches exhibit different physiological and pharmacological effects when administered to a subject. While recreational cannabinoid users may be prepared to tolerate a certain degree of variation in physiological effects, variation in physiological and pharmacological outcomes resulting from cannabinoid profile differences between preparation batches of a clinically administered drug is generally not acceptable. Furthermore, the production of plant extracts requires the growth and cultivation of Cannabis plants. The cultivation of Cannabis crops is subject to risks and uncertainties associated with climate and weather. In addition, there are commonly known legal and social challenges associated with the cultivation of Cannabis plants.

In response to the inherent shortcomings associated with plant sourced cannabinoid extracts, more recently, systems for the biosynthetic production of cannabinoid compounds in microorganisms and other cultured host cells have evolved. Several biosynthetic systems for cannabinoid compound have been reported (see e.g., WO2019071000, WO2018200888, WO2018148849, WO2019014490, US20180073043, US20180334692, and WO2019046941). Such biosynthetic systems can potentially avoid the need to grow a Cannabis crop, and provide more control over the produced cannabinoid profile and purity. Thus, biosynthetic production systems are more suitable for pharmaceutical production of cannabinoid compounds.

There remain, however, significant shortcomings associated with biosynthetic production systems for cannabinoid compounds. Notably, one limitation arises from the fact that cannabinoid compounds can be classified as lipophilic compounds, imparting, as will be understood by those of skill in the art, poor solubility in aqueous solutions. Thus, the solubility of CBD in water is less than 0.1 mg/ml, and the solubility of Δ⁹-THC is less than 0.01 mg/ml. Accordingly, it has been observed that in the operation of biosynthetic production systems, the cannabinoids synthesized by the cultured cells are generally poorly distributed within aqueous cellular environments, for example, the cellular cytosol, and instead, preferably associate with the lipidic cellular constituents of the cultured cells, including with the cellular or subcellular membranes, for example. The association of the biosynthesized cannabinoid compounds with the cellular membrane constituents is deemed to be particular undesirable, since the presence of cannabinoids within cellular or subcellular membranes can interfere with normal physiological membrane function of the cultured cells, and thereby induce cellular toxicity. In turn, this can substantially constrain growth of the cultured cells and their biosynthetic cannabinoid production capacity. The limited solubility in aqueous cell culture media may further also negatively impact the cannabinoid titer levels that can be achieved within culture media.

Furthermore, the lipophilic nature of cannabinoid compounds impedes the formulation of finished formulations containing cannabinoids. In particular, the lipophilic nature of cannabinoids represents a drawback in the preparation of cannabinoid containing finished formulations in which the cannabinoid compounds are homogenously dispersed. Thus, for example, due to the poor solubility of cannabinoid compounds, existing cannabinoid containing beverages frequently require shaking before use. In this respect, cannabinoid containing beverages can be said to compare unfavorably to alcohol containing beverages.

WO2017053574A1 (Vitality Biopharma, Inc.) discloses methods for preparing cannabinoid glycoside prodrugs through in vitro glycosyltransferase mediated glycosylation of cannabinoid molecules, specifically glycosylation mediated by the UDP-glycosyltransferases, UGT76G1 from Stevia rebaudiana, and Os03g0702000, from Oryza sativa.

WO2019014395A1 (Trait Biosciences, Inc.) discloses methods for preparing water soluble cannabinoids by contacting the cannabinoid with a suspension culture of genetically modified yeast cells that include a heterologous glycosyltransferase from Nicotiana tabacum (NtGT1; NtGT2; NtGT3; NtGT4; and NtGT5), Stevia rebaudiana (UGT76G1), or Arabidopsis thaliana. The reference does not disclose glycosyltransferase derived from Arabidopsis thaliana, or generation of a glycosylated cannabinoid generated in vivo by a yeast that includes a cannabinoid pathway.

There remains therefore a need in the art for improved processes to produce cannabinoid compounds, including, in particular, processes for the biosynthetic production of cannabinoid compounds. There also remains a need in the art for compounds and methods which can address the shortcomings associated with the lipophilic nature of cannabinoid compounds.

### SUMMARY

The following paragraphs are intended to introduce the detailed description and not intended to define or limit the subject matter of the present disclosure.

In at least one embodiment, the present disclosure provides methods for producing a glycosylated cannabinoid or a glycosylated cannabinoid precursor, the method comprising contacting under suitable reaction conditions: (a) a UDP-glycosyl transferase derived from Arabidopsis thaliana or Helianthus annuus; (b) a UDP-glycosyl substrate comprising a glycosyl group; and (c) a cannabinoid or a cannabinoid precursor comprising a hydroxyl group; whereby the glycosyl group is transferred to the hydroxyl group to form the glycosylated cannabinoid or the glycosylated cannabinoid precursor. In at least one embodiment, the UDP-glycosyl transferase comprises an amino acid sequence having at least 90% identity to a sequence selected from SEQ ID NO: 2, 4, 6, 8, 10, 12, 14, 16, or 18.

In at least one embodiment of the methods of the present disclosure, the cannabinoid is selected from cannabigerolic acid (CBGA), cannabigerol (CBG), cannabidiolic acid (CBDA), cannabidiol (CBD), Δ9-tetrahydrocannabinolic acid (Δ9-THCA), Δ9-tetrahydrocannabinol (Δ9-THC), Δ8-tetrahydrocannabinolic acid (Δ8-THCA), Δ8-tetrahydrocannabinol (Δ8-THC), cannabichromenic acid (CBCA), cannabichromene (CBC), cannabinolic acid (CBNA), cannabinol (CBN), cannabidivarinic acid (CBDVA), cannabidivarin (CBDV), Δ9-tetrahydrocannabivarinic acid (Δ9-THCVA), Δ9-tetrahydrocannabivarin (Δ9-THCV), cannabidibutolic acid (CBDBA), cannabidibutol (CBDB), Δ9-tetrahydrocannabutolic acid (Δ9-THCBA), Δ9-tetrahydrocannabutol (Δ9-THCB), cannabidiphorolic acid (CBDPA), cannabidiphorol (CBDP), Δ9-tetrahydrocannabiphorolic acid (Δ9-THCPA), Δ9-tetrahydrocannabiphorol (Δ9-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), and cannabielsoin (CBE).

In at least one embodiment of the methods of the present disclosure, the cannabinoid precursor is selected from olivetolic acid, divarinic acid, 2-heptyl-4,6-dihydroxybenzoic acid, and 2-butyl-4,6-dihydroxybenzoic acid.

In at least one embodiment of the methods of the present disclosure, the cannabinoid comprises at least two hydroxyl groups.

In at least one embodiment of the methods of the present disclosure, the glycosylated cannabinoid comprises at least two glycosyl groups.

In at least one embodiment of the methods of the present disclosure, the glycosylated cannabinoid is a compound of structural formula (I):

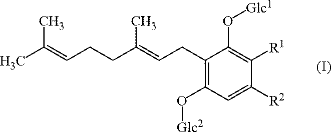

wherein,
R¹ is H or COOH;
R² is a C2-C7 alkyl chain; and
at least one of Glc¹ and Glc² is the glycosyl group, and if either of Glc¹ or Glc² is not a glycosyl group then it is H.

In at least one embodiment of the methods of the present disclosure, the glycosylated cannabinoid is a compound of structural formula (ll):

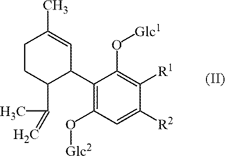

wherein,
R¹ is H or COOH;
R² is a C2-C7 alkyl chain; and
at least one of Glc¹ and Glc² is the glycosyl group, and if either of Glc¹ or Glc² is not a glycosyl group then it is H.

In at least one embodiment of the methods of the present disclosure, the glycosylated cannabinoid is a compound of structural formula (III):

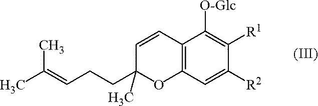

wherein,
R¹ is H or COOH;
R² is a C2-C7 alkyl chain; and
GIc is the glycosyl group.

In at least one embodiment of the methods of the present disclosure, the glycosylated cannabinoid is a compound of structural formula (IV):

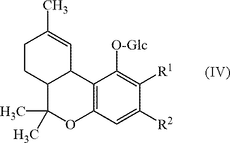

wherein,
R¹ is H or COOH;
R² is a C2-C7 alkyl chain; and
GIc is the glycosyl group.

In at least one embodiment of the methods of the present disclosure, the glycosylated cannabinoid precursor is a compound of structural formula (V):

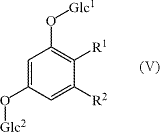

wherein,
R¹ is H or COOH;
R² is a C2-C7 alkyl chain; and
at least one of Glc¹ and Glc² is a glycosyl group, and if either of Glc¹ or Glc² is not a glycosyl group then it is H.

In at least one embodiment of the methods of the present disclosure, the glycosyl group, GIc, is a moiety of structural formula (VI):

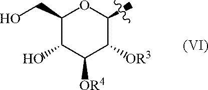

wherein, R³ is H, β-D-glucopyranosyl, or 3-O-β-D-glucopyranosyl-β-D-glucopyranosyl; and R⁴ is H, β-D-glucopyranosyl, or 3-O-β-D-glucopyranosyl-β-D-glucopyranosyl.

In at least one embodiment of the methods of the present disclosure, the glycosyl group (Glc) of the glycosylated cannabinoid is selected from a mono-saccharide, a disaccharide, and a tri-saccharide.

In at least one embodiment of the methods of the present disclosure, the UDP-glycosyl substrate is selected from UDP-glucose, UDP-galactose, UDP-xylose, UDP-glucuronic acid, UDP-N-acetylglucosamine, UDP-N-acetylgalactosamine, GDP-fucose, GDP-mannose, CMP-sialic acid, and a mixture thereof.

In at least one embodiment of the methods of the present disclosure, the glycosyl group comprises a glucosyl group, a galactosyl group, a xylosyl group, a glucuronic acid group, an N-acetylglucosyl group, an N-acetylgalactosyl group, a fucosyl group, a mannosyl group, a sialic acid group, an arabinosyl group, a rhamnosyl group, or a combination thereof.

In one embodiment, the method can comprise contacting the cannabinoid compound with the glycosyl group containing compound and the glycosyl transferase under in vitro conditions.

In at least one embodiment of the methods of the present disclosure, the contacting under suitable reaction conditions comprises in vivo conditions, wherein the in vivo conditions comprise growing a recombinant host cell comprising a heterologous nucleic acid that encodes the UDP-glycosyl transferase under conditions in which the cell expresses the UDP-glycosyl transferase. In at least one embodiment, the heterologous nucleic acid encodes an amino acid sequence having at least 90% identity to a sequence selected from SEQ ID NO: 2, 4, 6, 8, 10, 12, 14, 16, or 18. In at least one embodiment, the heterologous nucleic acid comprises a sequence having at least 90% identity to a sequence selected from SEQ ID NO: 1, 3, 5, 7, 9, 11, 13, 15, or 17.

In at least one embodiment of the method, wherein the method comprises growing a recombinant host cell, the recombinant host cell further comprises a pathway capable of producing the cannabinoid or the cannabinoid precursor; optionally, wherein the pathway comprises enzymes capable of converting hexanoic acid to olivetolic acid. In at least one embodiment, the pathway further comprises an enzyme capable of converting olivetolic acid and geranyldiphosphate to CBGA.

In at least one embodiment of the method comprising a recombinant host cell with a pathway capable of producing the cannabinoid or the cannabinoid precursor, the pathway comprises enzymes capable of catalyzing reactions (i) - (iii):

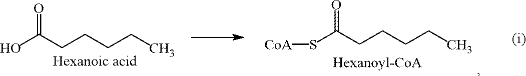

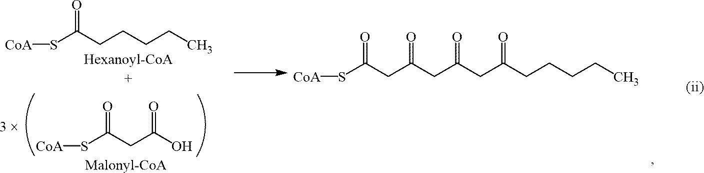

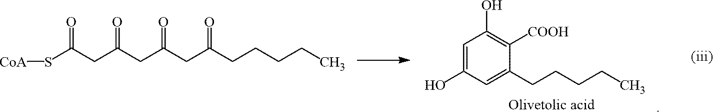

In at least one embodiment of the method comprising a recombinant host cell with a pathway capable of producing the cannabinoid or the cannabinoid precursor, the pathway further comprises and enzyme capable of catalyzing reaction (iv):

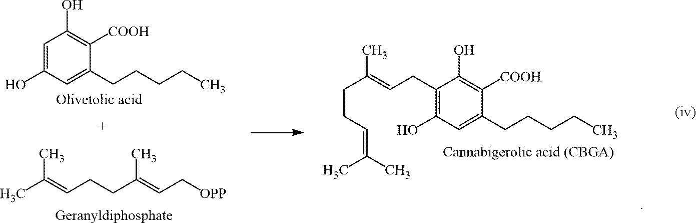

In at least one embodiment of the method comprising a recombinant host cell with a pathway capable of producing the cannabinoid or the cannabinoid precursor, the pathway comprises at least the following enzymes: AAE, OLS, and OAC; optionally, wherein the enzymes AAE, OLS, and OAC have an amino acid sequence of at least 90% identity to SEQ ID NO: 82 (AAE), SEQ ID NO: 84 (OLS), and SEQ ID NO: 86 (OAC), respectively. In at least one embodiment, the pathway further comprises the enzyme PT4; optionally, wherein the enzyme PT4 has an amino acid sequence of at least 90% identity to SEQ ID NO: 88 or 90.

In at least one embodiment of the method comprising a recombinant host cell with a pathway capable of producing the cannabinoid or the cannabinoid precursor, the pathway further comprises an enzyme capable of catalyzing the conversion of CBGA to Δ⁹-THCA, CBDA, and/or CBCA.

In at least one embodiment of the method comprising a recombinant host cell with a pathway capable of producing the cannabinoid or the cannabinoid precursor, the pathway further comprises an enzyme capable of catalyzing a reaction (v), (vi), and/or (vii):

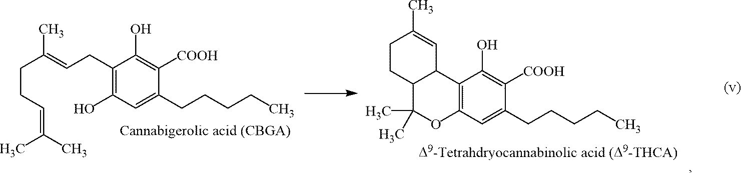

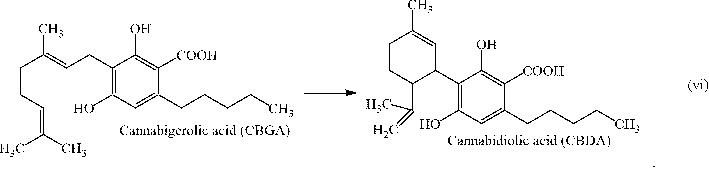

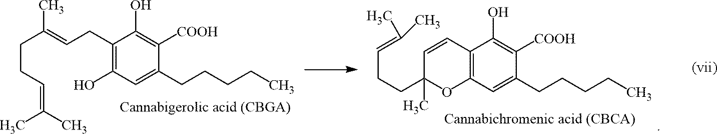

In at least one embodiment of the method comprising a recombinant host cell with a pathway capable of producing the cannabinoid or the cannabinoid precursor, the pathway further comprises: THCA synthase, CBDA synthase, and/or CBCA synthase; optionally, wherein the pathway comprises a CBDA synthase having an amino acid sequence of at least 90% identity to SEQ ID NO: 92 or 94.

In at least one embodiment of the method, wherein the method comprises growing a recombinant host cell with a pathway capable of producing the cannabinoid or the cannabinoid precursor, the method further comprises recovering the glycosylated cannabinoid or glycosylated precursor.

In at least one embodiment of the method comprising growing a recombinant host cell with a pathway capable of producing the cannabinoid or the cannabinoid precursor, the host cell is a microbial cell; optionally, the host cell is a cell derived from a source selected from: Saccharomyces cerevisiae, Escherichia coli, Yarrowia lipolytica, and Pichia pastoris.

In at least one embodiment, the present disclosure provide a recombinant host cell comprising: (a) a pathway capable of producing a cannabinoid or a cannabinoid precursor; and (b) a heterologous nucleic acid that encodes a UDP-glycosyl transferase derived from Arabidopsis thaliana or Helianthus annuus; wherein the host cell is capable of producing a glycosylated cannabinoid and/or a glycosylated cannabinoid precursor. In at least one embodiment, the heterologous nucleic acid encodes an amino acid sequence having at least 90% identity to a sequence selected from SEQ ID NO: 2, 4, 6, 8, 10, 12, 14, 16, or 18. In at least one embodiment, the heterologous nucleic acid comprises a sequence having at least 90% identity to a sequence selected from SEQ ID NO: 1, 3, 5, 7, 9, 11, 13, 15, or 17.

In at least one embodiment of the recombinant host cell, the pathway comprises enzymes capable of converting hexanoic acid to olivetolic acid. In at least one embodiment, the pathway further comprises an enzyme capable of converting olivetolic acid and geranyldiphosphate to CBGA.

In at least one embodiment of the recombinant host cell, the pathway comprises enzymes capable of catalyzing reactions (i) - (iii):

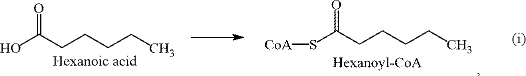

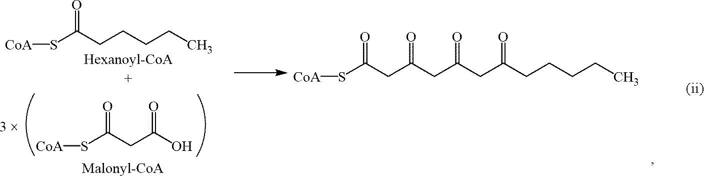

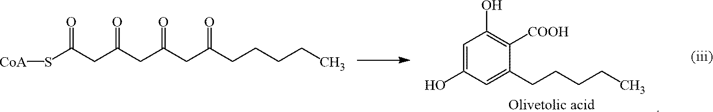

In at least one embodiment, the pathway further comprises and enzyme capable of catalyzing reaction (iv):

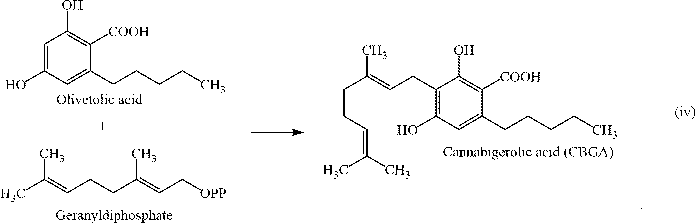

In at least one embodiment of the recombinant host cell, the pathway comprises at least the following enzymes: AAE, OLS, and OAC; optionally, wherein the enzymes AAE, OLS, and OAC have an amino acid sequence of at least 90% identity to SEQ ID NO: 82 (AAE), SEQ ID NO: 84 (OLS), and SEQ ID NO: 86 (OAC), respectively. In at least one embodiment, the pathway further comprises the enzyme PT4; optionally, wherein the enzyme PT4 has an amino acid sequence of at least 90% identity to SEQ ID NO: 88 or 90.

In at least one embodiment of the recombinant host cell, the pathway further comprises an enzyme capable of catalyzing the conversion of CBGA to Δ⁹-THCA,CBDA, and/or CBCA.

In at least one embodiment of the recombinant host cell, the pathway further comprises an enzyme capable of catalyzing a reaction (v), (vi), and/or (vii):

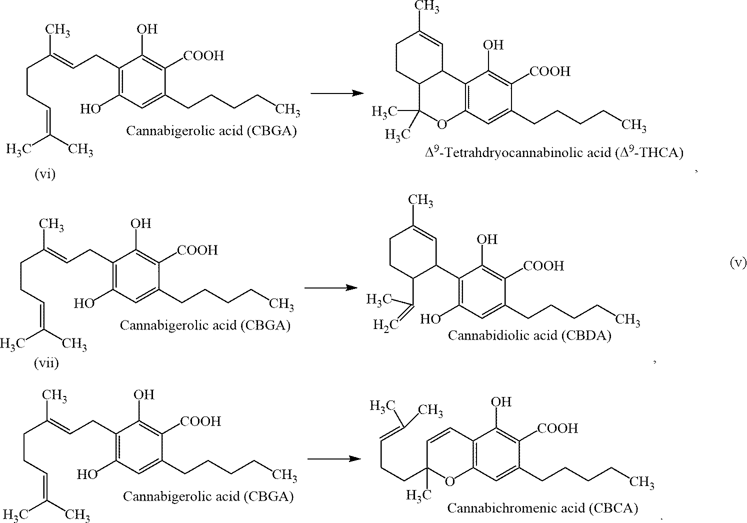

In at least one embodiment of the recombinant host cell, the pathway further comprises: THCA synthase, CBDA synthase, and/or CBCA synthase; optionally, wherein the pathway comprises a CBDA synthase having an amino acid sequence of at least 90% identity to SEQ ID NO: 92 or 94.

In at least one embodiment of the recombinant host cell, the cell is capable of producing a glycosylated cannabinoid of any one of structural formulae (I), (II), (III), and/or (IV), or a glycosylated cannabinoid precursor of structural formula (V), as those formulae are described elsewhere herein.

In at least one embodiment, the present disclosure also provides a composition comprising a glycosylated cannabinoid compound produced in accordance with any one of the methods of the present disclosure. Accordingly, the present disclosure provides a composition comprising a glycosylated cannabinoid of any one of structural formulae (I), (II), (III), and/or (IV), or a glycosylated cannabinoid precursor of structural formula (V), as those formulae are described elsewhere herein. In at least one embodiment, the composition comprising a glycosylated cannabinoid compound produced in accordance with any one of the methods of the present disclosure is a pharmaceutical composition.

In at least one embodiment, the present disclosure also provides a use of a glycosylated cannabinoid compound produced in accordance with any one of the methods of the present disclosure in as an ingredient in a cosmetic, food, beverage, or pharmaceutical composition.

Other features and advantages will become apparent from the following detailed description. It should be understood, however, that the detailed description, while indicating preferred implementations of the disclosure, are given by way of illustration only, since various changes and modifications within the spirit and scope of the disclosure will become apparent to those of skill in the art from the detailed description.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts an exemplary UDP-glycosylase catalyzed cannabinoid glycosylation reaction, the enzymatic glycosylation of the cannabinoid, CBD with the substrate, UDP-glucose to produce mono-glucosylated CBD.

FIG. 2 depicts an exemplary pathway capable of converting hexanoic acid to CBGA. The four enzymes catalyzing the steps in the biosynthetic pathway, AAE, OLS, OAC, PT, are indicated.

FIG. 3 depicts an exemplary pathway capable of catalyzing the conversion of CBGA to Δ⁹-THCA,CBDA, and/or CBCA. The various enzymes, CBDAs, THCAs and CBCAs, capable of catalyzing the conversions in the biosynthetic pathway are indicated.

FIG. 4A and FIG. 4B are images of agarose gels showing expression of heterologous UGT genes transformed in recombinant yeast host cells cDNAs as described in Example 1. FIG. 4A gel lanes: (1) Empty vector control, (2) AtUGT73C6, (3) AtUGT73B4, (4) AtUGT71D1, (5) HaUGT76G1-L, (6) AtUGT76E12, (7) AtUGT88A1, (8) At5g49690, (9) AtUGT76C4, (10) negative control. FIG. 4B gel lanes: (1) Empty vector control, (2) SrUGT76G1, (3) AtUGT85A3, (4) AtUGT79B1, (5) At5g65550, (6) AtUGT76B1, (7) AtUGT76D1, (8) CsUGT75B2, (9) CsUGT73B4, (10) CsUGT73B1, (11) CsUGT71D1_DN11028, (12) CsUGT71D1_DN4828, (13) negative control, (14) CsUGT73C6.

FIG. 5 depicts plots showing reduction in the amount of CBDA in nine different strains BL21 (DE3) expressing different UDP-glycosyl transferases (UGTs) as described in Example 3. The values shown are averages from triplicates, and the error bars represent standard deviations. * indicates p<0.05 (T-test).

### DETAILED DESCRIPTION

Various methods, compositions, and systems of the present disclosure are described in greater detail below to provide exemplary embodiments of the claimed subject matter. None of the exemplary embodiments described herein are intended to limit the claimed subject matter and any claimed subject matter may cover methods, compositions, and systems that differ from those described below. The claimed subject matter is not limited to compositions, processes or systems having all of the features of any one composition, system or process described below or to features common to multiple or all of the methods, compositions, or systems described below. The detailed description may also include methods, compositions, or systems that are not within the claimed subject matter. Any subject matter disclosed herein and not within the subject matter of the claims of the present disclosure may be within the claimed subject matter of, for example, a continuing patent application, and the applicant(s), inventor(s) or owner(s) do not intend to abandon, disclaim or dedicate to the public any such subject matter by its disclosure in this document.

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.” Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. For example, a range of 1 to 5 includes 1, 1.5, 2, 2.75, 3, 3.90, 4, and 5. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by context. Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Generally, the nomenclature used herein and the techniques and procedures described herein include those that are well understood and commonly employed by those of ordinary skill in the art, such as the common techniques and methodologies described in Sambrook et al., Molecular Cloning-A Laboratory Manual (2nd Ed.), Vols. 1-3, Cold Spring Harbor Laboratory, Cold Spring Harbor, N.Y., 1989 (hereinafter “Sambrook”); Current Protocols in Molecular Biology, F. M. Ausubel et al., eds., Current Protocols, a joint venture between Greene Publishing Associates, Inc. and John Wiley & Sons, Inc. (supplemented through 2011) (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### Definitions

“Cannabinoid” refers to a compound that acts on cannabinoid receptor, and is intended to include the endocannabinoid compounds that are produced naturally in animals, the phytocannabinoid compounds produced naturally in Cannabis plants, and the synthetic cannabinoids compounds. Exemplary cannabinoids of the present disclosure include those compounds listed in Table 1 (below).

“Cannabinoid precursor compound”, as used herein, refers to a chemical compound that may serve as a chemical precursor, including cyclic carboxylic acid compounds, which upon chemical conversion thereof form a cannabinoid compound. Cannabinoid precursor compounds include without limitation hexanoic acid, hexanoyl-CoA, C₁₂-tetraketide, and olivetolic acid.

“Glycosyl group,” or “glycosyl moiety,” as used herein, refers to a saccharide group, such as a mono-, di-, tri- oligo-, or a poly-saccharide group, which is bonded to a compound through its anomeric carbon in either the a- or the β-conformation. Exemplary glycosyl groups include monosaccharide groups of various ring structures, including pentosyl, hexosyl, and heptosyl groups, and can include well-known saccharide groups such as glucosyl, glucuronic acid, galactosyl, fucosyl, xylose, arabinose, and rhamnose groups. A glycosyl group can be unsubstituted or optionally substituted with various groups. Exemplary optional substitutions of glycosyl groups may include lower alkyl, lower alkoxy, acyl, carboxy, carboxyamino, amino, acetamido, halo, thio, nitro, keto, and phosphatyl groups, wherein the substitution may be at one or more positions on the saccharide. Also included within the term glycosyl group are further stereoisomers, optical isomers, anomers, and epimers of the glycosyl group. Thus, a hexose group, for example, can be either an aldose or a ketose group, can be of D- or L-configuration, can assume either an a or β conformation, and can be a dextro- or levo-rotatory with respect to plane-polarized light.

“Glycosylated cannabinoid,” as used herein, refers to a cannabinoid compound bonded to a glycosyl group through a glycosidic bond. Exemplary glycosylated cannabinoids of the present disclosure include, but are not limited to, the compounds of structural formulas (I), (Ia), (Ib), (II), (IIa), (IIb), (III), (IIIa), (IV), and (IVa), as disclosed herein.

“Glycosylated cannabinoid precursor,” as used herein, refers to a cannabinoid precursor compound bonded to a glycosyl group through a glycosidic bond. Exemplary glycosylated cannabinoid precursors of the present disclosure include, but are not limited to, the compounds of structural formulas (V), (Va) and (Vb) as disclosed herein

“UDP glycosyl transferase,” or “UGT” as used herein, refers to an enzyme having uridine 5′-diphospho glycosyl transferase activity, and can comprise a sequence of amino acid residues which is (i) substantially identical to the amino acid sequences constituting any UDP transferase polypeptide set forth herein, including, but not limited to, polypeptides having an amino acid sequence of any one of SEQ ID NO: 2, 4, 6, 8, 10, 12, 14, 16, or 18, or (ii) encoded by a nucleic acid sequence capable of hybridizing under at least moderately stringent conditions to any nucleic acid sequence encoding any UDP glycosyl set forth herein, but for the use of synonymous codons.

The terms “nucleic acid sequence encoding a UDP glycosyl transferase”, as used herein, refers to any and all nucleic acid sequences encoding a UDP glycosyl transferase polypeptide, including, for example, a nucleotide sequence of any one of SEQ ID NO: 1, 3, 5, 7, 9, 11, 13, 15, or 17. Nucleic acid sequences encoding a UDP glycosyl transferase polypeptide further include any and all nucleic acid sequences which (i) encode polypeptides that are substantially identical to the UDP glycosyl transferase polypeptide sequences set forth herein; or (ii) hybridize to any UDP glycosyl transferase nucleic acid sequences set forth herein under at least moderately stringent hybridization conditions or which would hybridize thereto under at least moderately stringent conditions but for the use of synonymous codons.

“Pathway” refers an ordered sequence of enzymes that act in a linked series to convert an initial substrate molecule into final product molecule. As used herein, “pathway” is intended to encompass naturally-occurring pathways and non-naturally occurring, recombinant pathways. Accordingly, a pathway of the present disclosure can include a series of enzymes that are naturally-occurring and/or non-naturally occurring, and can include a series of enzymes that act in vivo or in vitro.

“Pathway capable of producing a cannabinoid” refers to a pathway that can convert an initial substrate molecule, such as hexanoic acid, into a final product molecule that is a cannabinoid, such as cannabigerolic acid (CBGA). For example, the four enzymes AAE, OLS, OAC, and PT4 which convert hexanoic acid to CBGA, form a pathway capable of producing a cannabinoid.

“Conversion” as used herein refers to the enzymatic conversion of the substrate(s) to the corresponding product(s). “Percent conversion” refers to the percent of the substrate that is converted to the product within a period of time under specified conditions. Thus, the “enzymatic activity” or “activity” of an enzymatic conversion can be expressed as “percent conversion” of the substrate to the product.

“Substrate” as used herein in the context of an enzyme mediated process refers to the compound or molecule acted on by the enzyme.

“Product” as used herein in the context of an enzyme mediated process refers to the compound or molecule resulting from the activity of the enzyme.

“Host cell” as used herein refers to a cell capable of being functionally modified with recombinant nucleic acids and functioning to express recombinant products, including polypeptides and compounds produced by activity of the polypeptides.

“Nucleic acid,” or “polynucleotide” as used herein interchangeably to refer to two or more nucleosides that are covalently linked together. The nucleic acid may be wholly comprised ribonucleosides (e.g., RNA), wholly comprised of 2′-deoxyribonucleotides (e.g., DNA) or mixtures of ribo- and 2′-deoxyribonucleosides. The nucleoside units of the nucleic acid can be linked together via phosphodiester linkages (e.g., as in naturally occurring nucleic acids), or the nucleic acid can include one or more non-natural linkages (e.g., phosphorothioester linkage). Nucleic acid or polynucleotide is intended to include single-stranded or double-stranded molecules, or molecules having both single-stranded regions and double-stranded regions. Nucleic acid or polynucleotide is intended to include molecules composed of the naturally occurring nucleobases (i.e., adenine, guanine, uracil, thymine and cytosine), or molecules comprising that include one or more modified and/or synthetic nucleobases, such as, for example, inosine, xanthine, hypoxanthine, etc.

“Protein,” “polypeptide,” and “peptide” are used herein interchangeably to denote a polymer of at least two amino acids covalently linked by an amide bond, regardless of length or post-translational modification (e.g., glycosylation, phosphorylation, lipidation, myristilation, ubiquitination, etc.). As used herein “protein” or “polypeptide” or “peptide” polymer can include D- and L-amino acids, and mixtures of D- and L-amino acids.

“Naturally-occurring” or “wild-type” as used herein refers to the form as found in nature. For example, a naturally occurring nucleic acid sequence is the sequence present in an organism that can be isolated from a source in nature and which has not been intentionally modified by human manipulation.

“Recombinant,” “engineered,” or “non-naturally occurring” when used herein with reference to, e.g., a cell, nucleic acid, or polypeptide, refers to a material, or a material corresponding to the natural or native form of the material, that has been modified in a manner that would not otherwise exist in nature, or is identical thereto but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Nucleic acid derived from” as used herein refers to a nucleic acid having a sequence at least substantially identical to a sequence of found in naturally in an organism. For example, cDNA molecules prepared by reverse transcription of mRNA isolated from an organism, or nucleic acid molecules prepared synthetically to have a sequence at least substantially identical to, or which hybridizes to a sequence at least substantially identical to a nucleic sequence found in an organism.

“Coding sequence” refers to that portion of a nucleic acid (e.g., a gene) that encodes an amino acid sequence of a protein.

“Heterologous nucleic acid” as used herein refers to any polynucleotide that is introduced into a host cell by laboratory techniques, and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism’s genome. In some embodiments, the polynucleotides encoding the imine reductase enzymes may be codon optimized for optimal production from the host organism selected for expression.

“Preferred, optimal, high codon usage bias codons” refers to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; CodonW, John Peden, University of Nottingham; McInemey, J. O, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Control sequence” as used herein refers to all sequences, which are necessary or advantageous for the expression of a polynucleotide and/or polypeptide as used in the present disclosure. Each control sequence may be native or foreign to the nucleic acid sequence encoding a polypeptide. Such control sequences include, but are not limited to, a leader, a promoter, a polyadenylation sequence, a pro-peptide sequence, a signal peptide sequence, and a transcription terminator. At a minimum, control sequences typically include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” as used herein refers to a configuration in which a control sequence is appropriately placed (e.g., in a functional relationship) at a position relative to a polynucleotide sequence or polypeptide sequence of interest such that the control sequence directs or regulates the expression of the sequence of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Percentage of sequence identity,” “percent sequence identity,” “percentage homology,” or “percent homology” are used interchangeably herein to refer to values quantifying comparisons of the sequences of polynucleotides or polypeptides, and are determined by comparing two optimally aligned sequences over a comparison window, wherein the portion of the polynucleotide or polypeptide sequence in the comparison window may comprise additions or deletions (or gaps) as compared to the reference sequence for optimal alignment of the two sequences. The percentage values may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=-4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys, Madison Wis.), using default parameters provided.

“Reference sequence” refers to a defined sequence used as a basis for a sequence comparison. A reference sequence may be a subset of a larger sequence, for example, a segment of a full-length nucleic acid or polypeptide sequence. A reference sequence typically is at least 20 nucleotide or amino acid residue units in length, but can also be the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (or gaps) of 20 percent or less as compared to the reference sequence (which does not comprise additions or deletions) for optimal alignment of the two sequences.

“Substantial identity” or “substantially identical” refers to a polynucleotide or polypeptide sequence that has at least 70% sequence identity, at least 80% sequence identity, at least 85% sequence identity, at least 90% sequence identity, at least 95 % sequence identity, or at least 99% sequence identity, as compared to a reference sequence over a comparison window of at least 20 nucleoside or amino acid residue positions, frequently over a window of at least 30-50 positions, wherein the percentage of sequence identity is calculated by comparing the reference sequence to a sequence that includes deletions or additions which total 20 percent or less of the reference sequence over the window of comparison.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered imine reductase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Isolated” as used herein in reference to a molecule means that the molecule (e.g., cannabinoid, polynucleotide, polypeptide) is substantially separated from other compounds that naturally accompany it, e.g., protein, lipids, and polynucleotides. The term embraces nucleic acids which have been removed or purified from their naturally-occurring environment or expression system (e.g., host cell or in vitro synthesis).

“Substantially pure” refers to a composition in which a desired molecule is the predominant species present (i.e., on a molar or weight basis it is more abundant than any other individual macromolecular species in the composition), and is generally a substantially purified composition when the object species comprises at least about 50 percent of the macromolecular species present by mole or % weight.

“Recovered” as used herein in relation to an enzyme, protein, or cannabinoid compound, refers to a more or less pure form of the enzyme, protein, or cannabinoid.

The term “functional variant”, as used herein in reference to polynucleotides or polypeptides, refers to polynucleotides or polypeptides capable of performing the same function as a noted reference polynucleotide or polypeptide. Thus, for example, a functional variant of the polypeptide set forth in SEQ ID NO: 2, refers to a polypeptide capable of performing the same function as the polypeptide set forth in SEQ ID NO: 2. Functional variants include modified a polypeptide wherein, relative to a noted reference polypeptide, the modification includes a substitution, deletion or addition of one or more amino acids. In some embodiments, substitutions are those that result in a replacement of one amino acid with an amino acid having similar characteristics. Such substitutions include, without limitation (i) glutamic acid and aspartic acid; (i) alanine, serine, and threonine; (iii) isoleucine, leucine and valine, (iv) asparagine and glutamine, and (v) tryptophan, tyrosine and phenylalanine. Functional variants further include polypeptides having retained or exhibiting an enhanced cannabinoid biosynthetic bioactivity.

The term “chimeric”, as used herein in the context of nucleic acids, refers to at least two linked nucleic acids which are not naturally linked. Chimeric nucleic acids include linked nucleic acids of different natural origins. For example, a nucleic acid constituting a microbial promoter linked to a nucleic acid encoding a plant polypeptide is considered chimeric. Chimeric nucleic acids also may comprise nucleic acids of the same natural origin, provided they are not naturally linked. For example, a nucleic acid constituting a promoter obtained from a particular cell-type may be linked to a nucleic acid encoding a polypeptide obtained from that same cell-type, but not normally linked to the nucleic acid constituting the promoter. Chimeric nucleic acids also include nucleic acids comprising any naturally occurring nucleic acids linked to any non-naturally occurring nucleic acids.

The terms “substantially pure” and “isolated”, as may be used interchangeably herein describe a compound, e.g., a cannabinoid, polynucleotide or a polypeptide, which has been separated from components that naturally accompany it. Typically, a compound is substantially pure when at least 60%, more preferably at least 75%, more preferably at least 90%, 95%, 96%, 97%, or 98%, and most preferably at least 99% of the total material (by volume, by wet or dry weight, or by mole percent or mole fraction) in a sample is the compound of interest. Purity can be measured by any appropriate method, e.g., in the case of polypeptides, by chromatography, gel electrophoresis or HPLC analysis.

The term “in vivo”, as used herein, means within a cell, for example, within a microbial host cell, and can refer to a location for the performance of a reaction.

The term “in vitro”, as used herein, means outside a cell, for example, in a tube, a bottle, a dish, a microtiter plate, and the like, and can refer to a location for the performance of a reaction.

The term “recovered” as used herein in association with an enzyme, protein, a secondary metabolite or a cannabinoid, refers to a more or less pure form of the enzyme, protein, secondary metabolite, or cannabinoid.

### Methods of Preparing Glycosylated Cannabinolds and Glycosylated Cannabinoid Precursors Using UDP-Glycosyltransferases

The present disclosure relates to glycosylated cannabinoid and glycosylated cannabinoid precursor compounds and in vitro and in vivo methods for their preparation using recombinant glycosyltransferases derived from plant sources other than Stevia rebaudiana or Cannabis sativa. A surprising and unexpected technical effect of the present disclosure is that certain recombinant UDP-glycosyltransferases (UGTs) derived from Arabidopsis thaliana or Helianthus annuus can catalyze the transfer of a glycosyl group from a UDP-glycosyl substrate to a hydroxyl group of a cannabinoid or cannabinoid precursor to produce the corresponding glycosylated compounds. In particular, the UGTs derived from Arabidopsis thaliana or Helianthus annuus having an amino acid sequence of SEQ ID NO: 2, 4, 6, 8, 10, 12, 14, 16, or 18, when expressed recombinantly in eukaryotic (e.g., S. cerevisiae) or prokaryotic cells (e.g., E. coli) in the presence of cannabinoids or cannabinoid precursors resulted in production of the glycosylated compounds (e.g., mono- and di-glucosylated-olivetolic acid, mono- and di-glucosylated-CBGA, mono- and di-glucosylated-CBD). As described elsewhere herein, not all tested recombinant UGTs derived from A. thaliana (or S. rebaudiana, or C. sativa) are capable of producing glycosylated cannabinoids or cannabinoid precursors.

Accordingly, in at least one embodiment, the present disclosure provides a method of producing a glycosylated cannabinoid or a glycosylated cannabinoid precursor, the method comprising contacting under suitable reaction conditions: (a) a UDP-glycosyl transferase derived from Arabidopsis thaliana or Helianthus annuus; (b) a UDP-glycosyl substrate comprising a glycosyl group; and (c) a cannabinoid or a cannabinoid precursor comprising a hydroxyl group; whereby the glycosyl group is transferred to the hydroxyl group to form the glycosylated cannabinoid or the glycosylated cannabinoid precursor. In at least one embodiment, the UDP-glycosyl transferase comprises an amino acid sequence having at least 90% identity to a sequence selected from SEQ ID NO: 2, 4, 6, 8, 10, 12, 14, 16, or 18.

In general, the methods and compositions provided herein are useful in that they facilitate a efficient means for producing glycosylated cannabinoid and glycosylated precursor compounds. Such glycosylated compounds can avoid certain drawbacks associated with the corresponding non-glycosylated compounds. For example, the glycosylated cannabinoid compounds are useful in for preparing aqueous cannabinoids formulations, such as beverages, with improved solubility profiles. Additionally, the recombinant in vitro and in vivo methods of the present disclosure can avoid drawbacks associated with the production of glycosylated cannabinoid or glycosylated cannabinoid precursor compounds from natural plant extracts which often contain a mixture of components. Thus, the methods of the present disclosure can provide cannabinoid preparations with a superior cannabinoid profile. In particular, the methods of the present disclosure permit much tighter control over the cannabinoid profiles of different production batches. Therefore, comparative cannabinoid profiles of production batches can be much more similar, if not identical, than the cannabinoid profiles obtained when batches are prepared from plant extracts.

Furthermore, the methods of the present disclosure for preparation of glycosylated cannabinoids can avoid challenges associated with the lipophilic nature of cannabinoid compounds produced by known biosynthetic methods. For example, the methods of the present disclosure that produce glycosylated cannabinoids and cannabinoid precursors can reduce or avoid the cytotoxic effects often associated with the biosynthetic production of cannabinoid compounds in host cells. This in turn, can result in overall increased cannabinoid production capacity and yield of biosynthetic cannabinoid production systems.

Generally, the glycosylated cannabinoid and cannabinoid precursor compounds produced according to the methods of the present disclosure are useful inter alia as ingredients in the manufacture of cannabinoid containing formulations, including pharmaceutical, nutraceutical, cosmetic, food, or beverage compositions.

A wide range of cannabinoid and cannabinoid precursor compounds are suitable for glycosylation in accordance with the methods of the present disclosure, including those compounds having at least one hydroxyl group available for glycosylation. Accordingly, exemplary suitable cannabinoids and cannabinoid precursors for glycosylation include those provided in Table 1 below.

TABLE 1

Cannabinoid and cannabinoid precursor compounds

| | | |
|---|---|---|
| Compound Name | Abbrev. Name | Chemical Structure |
| cannabigerolic acid | CBGA | 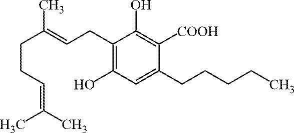 [SMILES: CCCCCc1cc(O)c(C/C=C(\C)CCC=C(C)C)c(O)c1C(=O)O] |
| cannabigerol | CBG | 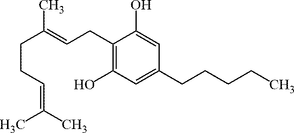 [SMILES: CCCCCc1cc(O)c(C/C=C(\C)CCC=C(C)C)c(O)c1] |
| Δ⁹-tetrahydrocannabinolic acid | Δ⁹-THCA | 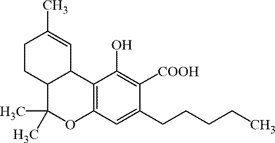 [SMILES: CCCCCc1cc2c(c(O)c1C(=O)O)C1C=C(C)CCC1C(C)(C)O2] |
| Δ⁹-tetrahydrocannabinol | Δ⁹-THC | 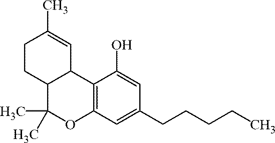 [SMILES: CCCCCc1cc(O)c2c(c1)OC(C)(C)C1CCC(C)=CC21] |
| Δ⁸-tetrahydrocannabinolic acid | Δ⁸-THCA | 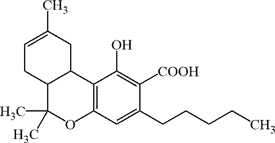 [SMILES: CCCCCc1cc2c(c(O)c1C(=O)O)C1CC(C)=CCC1C(C)(C)O2] |
| A⁸-tetrahydrocannabinol | Δ⁸-THC | 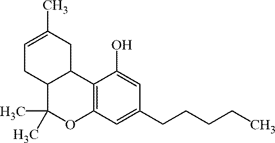 [SMILES: CCCCCc1cc(O)c2c(c1)OC(C)(C)C1CC=C(C)CC21] |
| cannabidiolic acid | CBDA | 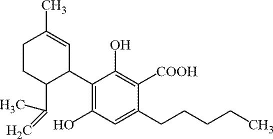 [SMILES: C=C(C)C1CCC(C)=CC1c1c(O)cc(CCCCC)c(C(=O)O)c1O] |
| cannabidiol | CBD | 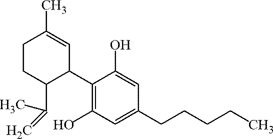 [SMILES: C=C(C)C1CCC(C)=CC1c1c(O)cc(CCCCC)cc1O] |
| cannabichromenic acid | CBCA | 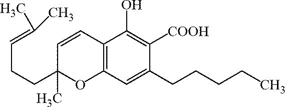 [SMILES: CCCCCc1cc2c(c(O)c1C(=O)O)C=CC(C)(CCC=C(C)C)O2] |
| cannabichromene | CBC | 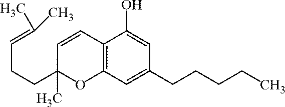 [SMILES: CCCCCc1cc(O)c2c(c1)OC(C)(CCC=C(C)C)C=C2] |
| cannabinolic acid | CBNA | 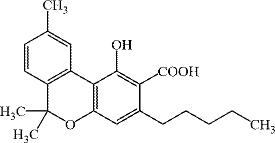 [SMILES: CCCCCc1cc2c(c(O)c1C(=O)O)-c1cc(C)ccc1C(C)(C)O2] |
| cannabinol | CBN | 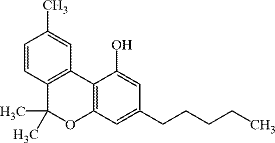 [SMILES: CCCCCc1cc(O)c2c(c1)OC(C)(C)c1ccc(C)cc1-2] |
| cannabidivarinic acid | CBDVA | 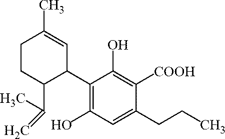 [SMILES: C=C(C)C1CCC(C)=CC1c1c(O)cc(CCC)c(C(=O)O)c1O] |
| cannabidivarin | CBDV | 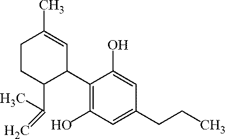 [SMILES: C=C(C)C1CCC(C)=CC1c1c(O)cc(CCC)cc1O] |
| Δ⁹-tetrahydrocannabivarinic acid | Δ⁹- THCVA | 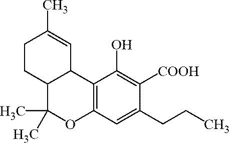 [SMILES: CCCc1cc2c(c(O)c1C(=O)O)C1C=C(C)CCC1C(C)(C)O2] |
| Δ⁹-tetrahydrocannabivarin | Δ⁹-THCV | 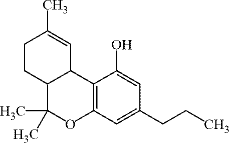 [SMILES: CCCc1cc(O)c2c(c1)OC(C)(C)C1CCC(C)=CC21] |
| Cannabidibutolic acid | CBDBA | 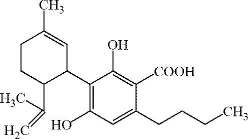 [SMILES: C=C(C)C1CCC(C)=CC1c1c(O)cc(CCCC)c(C(=O)O)c1O] |
| Cannabidibutol | CBDB | 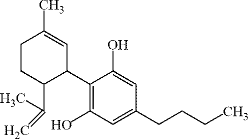 [SMILES: C=C(C)C1CCC(C)=CC1c1c(O)cc(CCCC)cc1O] |
| Δ⁹- tetrahydrocannabutolic acid | Δ⁹- THCBA | 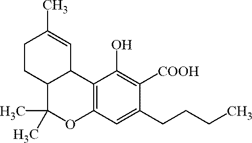 [SMILES: CCCCc1cc2c(c(O)c1C(=O)O)C1C=C(C)CCC1C(C)(C)O2] |
| Δ⁹- tetrahydrocannabutol | Δ⁹-THCB | 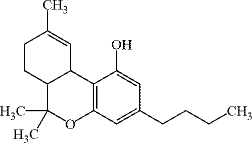 [SMILES: CCCCc1cc(O)c2c(c1)OC(C)(C)C1CCC(C)=CC21] |
| Cannabidiphorolic acid | CBDPA | 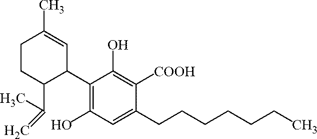 [SMILES: C=C(C)C1CCC(C)=CC1c1c(O)cc(CCCCCCC)c(C(=O)O)c1O] |
| Cannabidiphorol | CBDP | 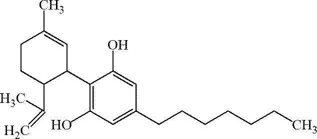 [SMILES: C=C(C)C1CCC(C)=CC1c1c(O)cc(CCCCCCC)cc1O] |
| Δ⁹- tetrahydrocannabiphorolic acid | Δ⁹- THCPA | 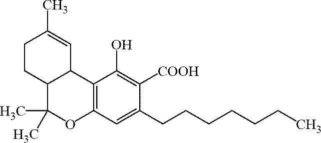 [SMILES: CCCCCCCc1cc2c(c(O)c1C(=O)O)C1C=C(C)CCC1C(C)(C)O2] |
| A⁹- tetrahydrocannabiphorol | Δ⁹-THCP | 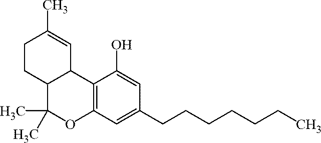 [SMILES: CCCCCCCc1cc(O)c2c(c1)OC(C)(C)C1CCC(C)=CC21] |
| cannabichromevarinic acid | CBCVA | 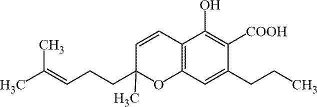 [SMILES: CCCc1cc2c(c(O)c1C(=O)O)C=CC(C)(CCC=C(C)C)O2] |
| cannabichromevarin | CBCV | 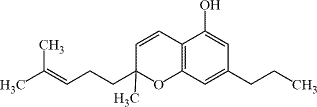 [SMILES: CCCc1cc(O)c2c(c1)OC(C)(CCC=C(C)C)C=C2] |
| cannabigerovarinic acid | CBGVA | 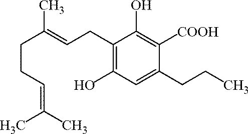 [SMILES: CCCc1cc(O)c(C/C=C(\C)CCC=C(C)C)c(O)c1C(=O)O] |
| cannabigerovarin | CBGV | 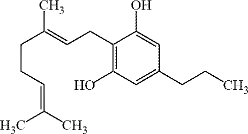 [SMILES: CCCc1cc(O)c(C/C=C(\C)CCC=C(C)C)c(O)c1] |
| cannabicyclolic acid | CBLA | 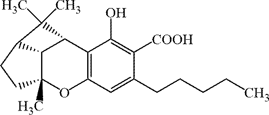 |
| cannabicyclol | CBL | 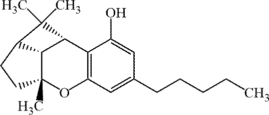 |
| cannabielsoinic acid | CBEA | 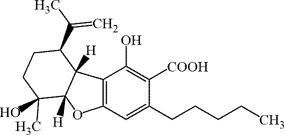 [SMILES: C=C(C)[C@@H]1CC[C@](C)(O)[C@H]2Oc3cc(CCCCC)c(C(=O)O)c(O)c3[C@@H]12] |
| cannabielsoin | CBE | 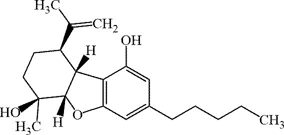 [SMILES: C=C(C)[C@@H]1CC[C@](C)(O)[C@H]2Oc3cc(CCCCC)cc(O)c3[C@@H]12] |
| 2-heptyl-4,6-dihydroxybenzoic acid | | 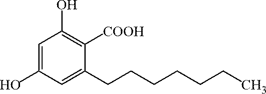 [SMILES: CCCCCCCc1cc(O)cc(O)c1C(=O)O] |
| olivetolic acid | OA | 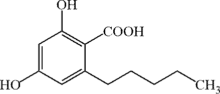 [SMILES: CCCCCc1cc(O)cc(O)c1C(=O)O] |
| 2-butyl-4,6-dihydroxybenzoic acid | | 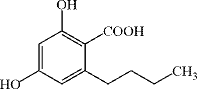 |
| divarinic acid | DA | 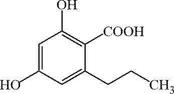 |

Accordingly, in at least one embodiment, the cannabinoid glycosylated according in the methods of the present disclosure can include a cannabinoid selected from cannabigerolic acid (CBGA), cannabigerol (CBG), cannabidiolic acid (CBDA), cannabidiol (CBD), Δ9-tetrahydrocannabinolic acid (Δ9-THCA), Δ9-tetrahydrocannabinol (Δ9-THC), Δ8-tetrahydrocannabinolic acid (Δ8-THCA), Δ8-tetrahydrocannabinol (Δ8-THC), cannabichromenic acid (CBCA), cannabichromene (CBC), cannabinolic acid (CBNA), cannabinol (CBN), cannabidivarinic acid (CBDVA), cannabidivarin (CBDV), Δ9-tetrahydrocannabivarinic acid (Δ9-THCVA), Δ9-tetrahydrocannabivarin (Δ9-THCV), cannabidibutolic acid (CBDBA), cannabidibutol (CBDB), Δ9-tetrahydrocannabutolic acid (Δ9-THCBA), Δ9-tetrahydrocannabutol (Δ9-THCB), cannabidiphorolic acid (CBDPA), cannabidiphorol (CBDP), Δ9-tetrahydrocannabiphorolic acid (Δ9-THCPA), Δ9-tetrahydrocannabiphorol (Δ9-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), and cannabielsoin (CBE).

Further, in at least one embodiment, the cannabinoid precursor glycosylated according to the methods of the present disclosure can include a cannabinoid precursor selected from olivetolic acid, divarinic acid, 2-heptyl-4,6-dihydroxybenzoic acid, and 2-butyl-4,6-dihydroxybenzoic acid.

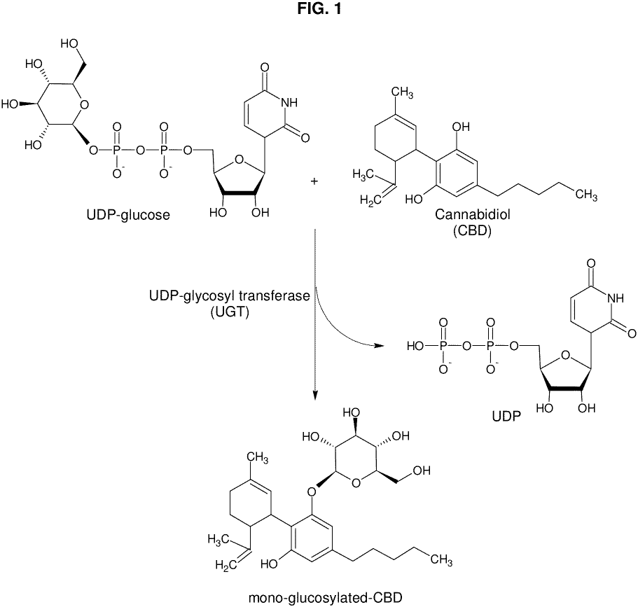

UDP-glycosyl substrates that may be used in accordance with the methods and compositions of the present disclosure can include any UDP-glycosyl compound which can be accepted as a substrate by a UDP glycosyl transferase. As shown by the exemplary reaction depicted in FIG. 1, the UDP-glycosyl transferase (UGT) enzyme catalyzes transfer of the glycosyl group of a UDP-glycosyl substrate (e.g., UDP-glucose) to a cannabinoid acceptor substrate (e.g., CBD) via formation of a glycosidic bond to at least one hydroxyl group.

Referring further to FIG. 1, it should also be noted that the cannabinoid, CBD, represents an exemplary cannabinoid compound only. Other cannabinoid or cannabinoid precursor compounds that may be glycosylated using a UDP-glycosyl transferase according to the methods of the present disclosure can include any of the cannabinoids shown in Table 1.

As noted elsewhere herein, the suitable cannabinoid substrate comprises at least one hydroxyl residue that is available to accept the catalytic transfer of the glycosyl group of the substrate via formation of a glycosidic bond, however, in some embodiments where the cannabinoid comprises two free hydroxyl groups it is possible that the UGT can catalyze the transfer of two glycosyl groups to the cannabinoid.

As illustrated by the exemplary cannabinoid and cannabinoid precursor structures of Table 1, it is contemplated that the methods of the present disclosure can be used to glycosylate a range of compound structures at one or two free hydroxyl positions with a range of glycosyl groups. For example, in at least one embodiment of the methods of the present disclosure, the glycosylated cannabinoid is a compound having structural formula (I):

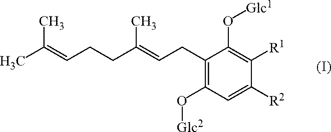

wherein, R¹ is H or COOH; R² is a C2-C7 alkyl chain; and at least one of two chemical groups denoted as Glc¹ and Glc² is a glycosyl group, and if either of Glc¹ or Glc² is not a glycosyl group then it is H. For example, glycosylated cannabinoids within this structural formula can include the mono-glucosylated CBGA and di-glucosylated CBGA compounds of structures (Ia) and (Ib) as shown below.

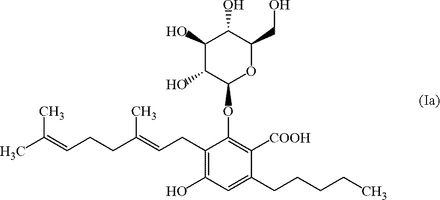 [SMILES: CCCCCc1cc(O)c(C/C=C(\C)CCC=C(C)C)c(O[C@@H]2O[C@H](CO)[C@@H](O)[C@H](O)[C@H]2O)c1C(=O)O]

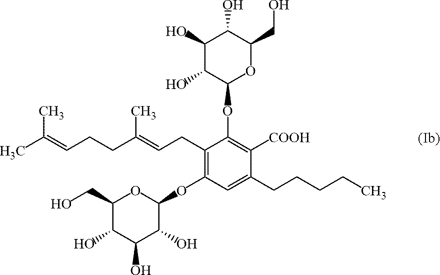 [SMILES: CCCCCc1cc(O[C@@H]2O[C@H](CO)[C@@H](O)[C@H](O)[C@H]2O)c(C/C=C(\C)CCC=C(C)C)c(O[C@@H]2O[C@H](CO)[C@@H](O)[C@H](O)[C@H]2O)c1C(=O)O]

In another example, in at least one embodiment of the methods of the present disclosure using UGT catalyzed glycosyl group transfer, the glycosylated cannabinoid prepared is a compound having structural formula (II):

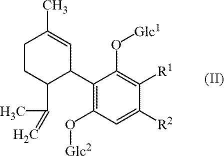

wherein, R¹ is H or COOH; R² is a C2-C7 alkyl chain; and wherein at least one of groups denoted as Glc¹ and Glc² is a glycosyl group. In embodiments, where only one of Glc¹ or Glc² is a glycosyl group then the other group denoted by Glc is a hydrogen (H). For example, glycosylated cannabinoids within this structural formula can include the mono-glucosylated CBD and di-glucosylated CBD compounds of structures (IIa) and (IIb) as shown below.

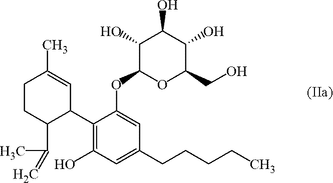 [SMILES: C=C(C)C1CCC(C)=CC1c1c(O)cc(CCCCC)cc1O[C@@H]1O[C@H](CO)[C@@H](O)[C@H](O)[C@H]1O]

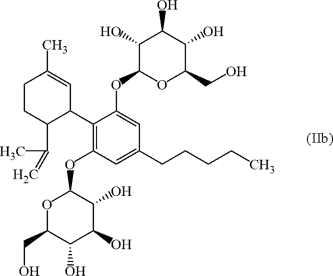 [SMILES: C=C(C)C1CCC(C)=CC1c1c(O[C@@H]2O[C@H](CO)[C@@H](O)[C@H](O)[C@H]2O)cc(CCCCC)cc1O[C@@H]1O[C@H](CO)[C@@H](O)[C@H](O)[C@H]1O]

In an example using a cannabinoid having only a single free hydroxyl group, in at least one embodiment of the methods of the present disclosure, the glycosylated cannabinoid prepared using UGT catalyzed glycosyl group transfer is a compound of structural formula (III):

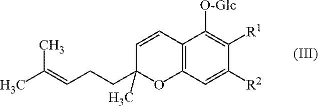

wherein, R¹ is H or COOH; R² is a C2-C7 alkyl chain; and the group denoted by GIc is a glycosyl group, such as a glucosyl moiety. For example, a glycosylated cannabinoid within this structural formula (III) can include glucosylated-CBCVA of structure (IIIa) below.

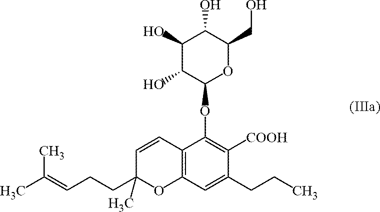 [SMILES: CCCc1cc2c(c(O[C@@H]3O[C@H](CO)[C@@H](O)[C@H](O)[C@H]3O)c1C(=O)O)C=CC(C)(CCC=C(C)C)O2]

In another example of using a cannabinoid having only a single free hydroxyl group, in at least one embodiment of the methods of the present disclosure, the glycosylated cannabinoid prepared using UGT catalyzed glycosyl group transfer is a compound of structural formula (IV):

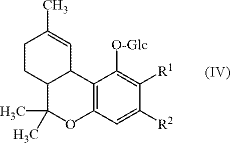

wherein, R¹ is H or COOH; R² is a C2-C7 alkyl chain; and Glc denotes a glycosyl group. For example, a glycosylated cannabinoid within this structural formula (IV) can include glucosylated-THC of structure (IVa) below.

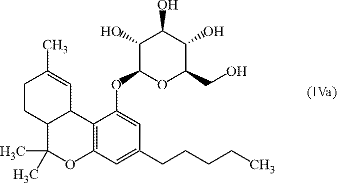 [SMILES: CCCCCc1cc(O[C@@H]2O[C@H](CO)[C@@H](O)[C@H](O)[C@H]2O)c2c(c1)OC(C)(C)C1CCC(C)=CC21]

In an example of using a cannabinoid precursor compound having two free hydroxyl groups, in at least one embodiment of the methods of the present disclosure, the glycosylated cannabinoid precursor prepared using UGT catalyzed glycosyl group transfer is a compound of structural formula (V):

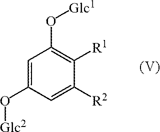

wherein, R¹ is H or COOH; R² is a C2-C7 alkyl chain; and at least one of groups denoted as Glc¹ and Glc² is a glycosyl group, and if either of Glc¹ or Glc² is not a glycosyl group then it is a hydrogen, H. For example, glycosylated cannabinoid precursor compounds within this structural formula can include the mono- and di-glucosylated olivetolic acid compounds of structures (Va) and (Vb) as shown below.

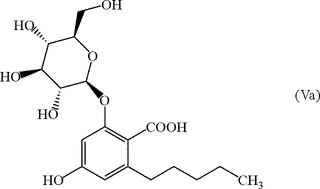 [SMILES: CCCCCc1cc(O)cc(O[C@@H]2O[C@H](CO)[C@@H](O)[C@H](O)[C@H]2O)c1C(=O)O]

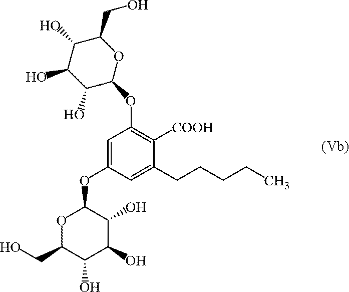 [SMILES: CCCCCc1cc(O[C@@H]2O[C@H](CO)[C@@H](O)[C@H](O)[C@H]2O)cc(O[C@@H]2O[C@H](CO)[C@@H](O)[C@H](O)[C@H]2O)c1C(=O)O]

The above shown exemplary mono- and di-glycosylated cannabinoid and cannabinoid precursor compounds of structures (Ia), (Ib), (IIa), (IIb), (IIIa), (IVa), (Va), and (Vb), comprise a glucosyl group that can be prepared in the methods of the present disclosure using a UGT enzyme as disclosed herein together with a UDP-glucose substrate. However, as is disclosed elsewhere herein and is known in the art, UGT enzymes are capable of catalyzing glycosyl group transfer from a range of UDP-glycosyl substrates to a cannabinoid or cannabinoid precursor as acceptor substrate. Accordingly, it is contemplated that in at least one embodiment of the methods of the present disclosure, the UDP-glycosyl substrate used is selected from UDP-glucose, UDP-galactose, UDP-xylose, UDP-glucuronic acid, UDP-N-acetylglucosamine, UDP-N-acetylgalactosamine, GDP-fucose, GDP-mannose, CMP-sialic acid, and a mixture thereof. Furthermore, in at least one embodiment of the methods of the present disclosure, the glycosyl group transferred to the cannabinoid or cannabinoid precursor acceptor substrate can include a glucosyl group, a galactosyl group, a xylosyl group, a glucuronic acid group, an N-acetylglucosyl group, an N-acetylgalactosyl group, a fucosyl group, a mannosyl group, a sialic acid group, an arabinosyl group, a rhamnosyl group, or a combination thereof.

In at least one embodiment of the methods of the present disclosure, the glycosyl group (Glc) of the glycosylated cannabinoid is selected from a mono-saccharide, a di-saccharide, and a tri-saccharide. For example, in at least one embodiment of the methods, the glycosyl group, Glc, of the glycosylated cannabinoid or glycosylated cannabinoid precursor is a moiety of structural formula (VI):

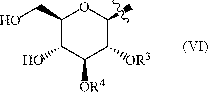

wherein, R³ is H, β-D-glucopyranosyl, or 3-O-β-D-glucopyranosyl-β-D-glucopyranosyl; and R⁴ is H or β-D-glucopyranosyl, or 3-O-β-D-glucopyranosyl-β-D-glucopyranosyl.

As noted elsewhere herein, the present disclosure provides methods for making glycosylated cannabinoids and glycosylated cannabinoid precursor compounds in vitro and in vivo using UDP-glycosyl transferases (UGTs) derived from the plants Arabidopsis thaliana and Helianthus annuus. Exemplary UGTs useful in the methods of the present disclosure comprise a polypeptide having any one of the amino acid sequences set forth in SEQ ID NO: 2, 4, 6, 8, 10, 12, 14, 16, or 18, or an amino acid sequence that is substantial identical thereto, for example at least 80%, at 85%, at least 90%, at least 95%, at least 96%, at least 97%, at least 98%, or at least 99% identical thereto; or a functional variant of any one of the amino acid sequences set forth in SEQ ID NO: 2, 4, 6, 8, 10, 12, 14, 16, or 18. UDP glycosyl transferase (UGT) polypeptide sequences of the present disclosure are summarized in Table 2 below and the accompanying Sequence Listing.

TABLE 2

UGT sequences of the present disclosure

| Source Organism | Annotation | Sequence | SEQ ID NO: |
|---|---|---|---|
| Helianthus annuus | HaUGT76G 1L | ATGGAGACCCAAACAGAAACCACCAACACCGTTCGCCGGAACCAGA GAATAATATTCTTCCCGTTACCATATCAAGGCCACATAAACCCAAT GCTCCAACTTGCCAATCTACTCTACTCCAAAGGCTTCAGTATCACC ATCCTCCACACCAACTTCAACAAGCCCAAAACATCCAACTACCCTC ACTTCACTTTCAAATTCATCCTGGACAACGATCCACACGACGAACG CTATTCCAATCTACCGTTACATGGCATGGGCGCTTTTAACCGCCTT TTCGTGTTCAACGAAGATGGTGCAGATGAATTGCGCCATGAACTTG AACTGTTAATGTTAGCTTCGAAAGAAGATGACGAACATGTATCGTG TTTAATCACCGATGCGCTTTGGCACTTCACGCAATCAGTCGCTGAC AGCCTTAACCTCCCACGGCTTGTTTTGAGGACAAGCAGCTTGTTTT GTTTTCTTGCTTATGCTTCATTTCCTGTTTTTGATGATCTTGGTTA CCTTAATCTTGCTGATCAAACACGTCTGGACGAACAAGTGGCTGAG TTTCCTATGTTGAAAGTGAGAGATATTATAAAGTTGGGCTTTAAGA GCTCGAAAGATTCTATTGGAATGATGCTTGGTAATATGGTGAAACA AACGAAAGCGTCTTTGGGTATTATCTTTAACTCGTTTAAGGAACTC GAAGAGCCGGAGGTTGAAACTGTTATCCGTGATATTCTGGCACCGA GTTTTCTGATACCATTTCCAAAGCATTTCACAGCGTCATCCAGCAG CTTACTAGACCAAGATCGAACCGTTTTTCCATGGTTAGACCAACAG CCGCCTAATTCCGTTTTGTATGTTAGTTTTGGTAGCACGACTGAAG TGGATGAGAAAGATTTCTTGGAAATAGCTCATGGGTTGGTTGATAG CGAGCAGACGTTTTTATGGGTGGTTCGACCTGGGTATGTCAAGGGT CCGATTTGGATCGAACTGTTGGATGATGGGTTCGTTGGTGAAAAGG | 1 |
| | | GGCGTATTGTGAAATGGGCTCCTCAGCAAGAAGTGCTAGCTCATGA AGCAATAGGTGCGTTTTGGACTCATAGTGGATGGAACTCGACATTG GAAAGCGTTTGTGAAGGTGTTCCTATGATAATGTCGCCTTTTATGG GCGATCAAGCGTTGAACGCTAGATACATGAGTGATGTTTCCAAGGT AGGGGTGTATTTGGGAAACGGGTGGGAAAGACGAGAGATAGCGAGT GCCATAAGGAAAGTAATGGTGGATGAAGAAGGAGAACACATTAGAG AGAATGCAAGAGATTTGAAACAAAAGGCAGATGATTCTTTAGTGAA GGGTGGGTCTTCCTATGAGTCATTAGAGTCTCTAGTTGCTTATATT TCTTCCTTTTAG | |
| Helianthus annuus | HaUGT76G 1L | METQTETTNTVRRNRRIIFFPLPYQGHINPMLQLANLLYSKGFSIT ILHTNFNKPKTSNYPHFTFKFILDNDPHDGRYSNLPLHGMGAFNRV FVFNEDGADELRHELELLMLASKEDDEHVSCLITDALWHFTQSVAD SLSLPRLVLRTSSLFSFLIYASIPLLDDRGYLSLSDNTMALILDGL GYHDLSDNKRLEEQVEEFPMLKVKDIVKMGFKREKDGAGGMIDNMV KQTKTSSGIIWNSFKELEESELETIRRDIPAPSFPIPFAKHFTASS SSLLEHDRSFFPWLDQQPPKSVVYVSFGSVAQVEEKHFMEMVHGLV DSKQLFLWVVRPGFVSGSTWLEPLPDGFPGERGRIVKWAPQQEVLG HEAIGAFWTHGGWNSTLESVCEGVPMICSPFWGDQPLDARYVSDVW KVGVYLENGWKREEITGAIRRVMTDEEMRERARVLKQKLDVSLMKG GSSYESVESLVAYVSSF | 2 |
| Arabidopsis thaliana | AtUGT73C6 | ATGGCTTTCGAAAAAAACAACGAACCTTTTCCTCTTCACTTTGTTC TCTTCCCTTTCATGGCTCAAGGCCACATGATTCCCATGGTTGATAT TGCAAGGCTCTTGGCTCAGCGAGGTGTGCTTATAACAATTGTCACG ACGCCTCACAATGCAGCAAGGTTCAAGAATGTCCTAAACCGTGCCA TTGAGTCTGGTTTGCCCATCAACCTAGTGCAAGTCAAGTTTCCATA TCAAGAAGCTGGTCTGCAAGAAGGACAAGAAAATATGGATTTGCTT ACCACGATGGAGCAGATAACATCTTTCTTTAAAGCGGTTAACTTAC TCAAAGAACCAGTCCAGAACCTTATTGAAGAGATGAGCCCGCGACC AAGCTGTCTAATCTCTGATATGTGTTTGTCGTATACAAGCGAAATC GCCAAGAAGTTCAAAATACCAAAGATCCTCTTCCATGGCATGGGTT GCTTTTGTCTTCTGTGTGTTAACGTTCTGCGCAAGAACCGTGAGAT CTTGGACAATTTAAAGTCTGATAAGGAGTACTTCATTGTTCCTTAT TTTCCTGATAGAGTTGAATTCACAAGACCTCAAGTTCCGGTGGAAA CATATGTTCCTGCAGGCTGGAAAGAGATCTTGGAGGATATGGTAGA AGCGGATAAGACATCTTATGGTGTTATAGTCAACTCATTTCAAGAG CTCGAACCTGCGTATGCCAAAGACTTCAAGGAGGCAAGGTCTGGTA AAGCATGGACCATTGGACCTGTTTCCTTGTGCAACAAGGTAGGAGT AGACAAAGCAGAGAGGGGAAACAAATCAGATATTGATCAAGATGAG TGCCTTGAATGGCTCGATTCTAAGGAACCGGGATCTGTGCTCTACG TTTGCCTTGGAAGTATTTGTAATCTTCCTCTGTCTCAGCTCCTTGA GCTGGGACTAGGCCTAGAGGAATCCCAAAGACCTTTCATCTGGGTC ATAAGAGGTTGGGAGAAATACAAAGAGTTAGTTGAGTGGTTCTCGG AAAGCGGCTTTGAAGATAGAATCCAAGATAGAGGACTTCTCATCAA AGGATGGTCCCCTCAAATGCTTATCCTTTCACATCCTTCTGTTGGA GGGTTCTTAACGCACTGCGGATGGAACTCGACTCTTGAGGGGATAA CTGCTGGTCTACCAATGCTTACATGGCCACTATTTGCAGACCAATT CTGCAACGAGAAACTGGTCGTACAAATACTAAAAGTCGGTGTAAGT GCCGAGGTTAAAGAGGTCATGAAATGGGGAGAAGAAGAGAAGATAG GAGTGTTGGTGGATAAAGAAGGAGTGAAGAAGGCAGTGGAAGAACT AATGGGTGAGAGTGATGATGCAAAAGAGAGAAGAAGAAGAGCCAAA GAGCTTGGAGAATCAGCTCACAAGGCTGTGGAAGAAGGAGGCTCCT CTCATTCTAATATCACTTTCTTGCTACAAGACATAATGCAACTAGC ACAGTCCAATAATTGA | 3 |
| Arabidopsis thaliana | AtUGT73C6 | MAFEKNNEPFPLHFVLFPFMAQGHMIPMVDIARLLAQRGVLITIVT TPHNAARFKNVLNRAIESGLPINLVQVKFPYQEAGLQEGQENMDLL TTMEQITSFFKAVNLLKEPVQNLIEEMSPRPSCLISDMCLSYTSEI AKKFKIPKILFHGMGCFCLLCVNVLRKNREILDNLKSDKEYFIVPY FPDRVEFTRPQVPVETYVPAGWKEILEDMVEADKTSYGVIVNSFQE LEPAYAKDFKEARSGKAWTIGPVSLCNKVGVDKAERGNKSDIDQDE CLEWLDSKEPGSVLYVCLGSICNLPLSQLLELGLGLEESQRPFIWV IRGWEKYKELVEWFSESGFEDRIQDRGLLIKGWSPQMLILSHPSVG GFLTHCGWNSTLEGITAGLPMLTWPLFADQFCNEKLVVQILKVGVS | 4 |
| | | AEVKEVMKWGEEEKIGVLVDKEGVKKAVEELMGESDDAKERRRRAK ELGESAHKAVEEGGSSHSNITFLLQDIMQLAQSNN | |
| Arabidopsis thaliana | AtUGT88A1 | ATGGGTGAAGAAGCTATAGTTCTGTATCCTGCACCACCAATAGGTC ACTTAGTGTCCATGGTTGAGTTAGGTAAAACCATCCTCTCCAAAAA CCCATCTCTCTCCATCCACATTATCTTAGTTCCACCGCCTTATCAG CCGGAATCAACCGCCACTTACATCTCCTCCGTCTCCTCCTCCTTCC CTTCAATAACCTTCCACCATCTTCCCGCCGTCACACCGTACTCCTC CTCCTCCACCTCTCGCCACCACCACGAATCTCTCCTCCTAGAGATC CTCTGTTTTAGCAACCCAAGTGTCCACCGAACTCTTTTCTCACTCT CTCGGAATTTCAATGTCCGAGCAATGATCATCGATTTCTTCTGCAC CGCCGTTTTAGACATCACCGCTGACTTCACGTTCCCGGTTTACTTC TTCTACACCTCTGGAGCCGCATGTCTCGCCTTTTCCTTCTATCTCC CGACCATCGACGAAACAACCCCCGGAAAAAACCTCAAAGACATTCC TACAGTTCATATCCCCGGCGTTCCTCCGATGAAGGGCTCCGATATG CCTAAGGCGGTGCTCGAACGAGACGATGAGGTCTACGATGTTTTTA TAATGTTCGGTAAACAGCTCTCGAAGTCGTCAGGGATTATTATCAA TACGTTTGATGCTTTAGAAAACAGAGCCATCAAGGCCATAACAGAG GAGCTCTGTTTTCGCAATATTTATCCAATTGGACCGCTCATTGTAA ACGGAAGAATCGAAGATAGAAACGACAACAAGGCAGTTTCTTGTCT CAATTGGCTGGATTCGCAGCCGGAAAAGAGTGTTGTGTTTCTCTGT TTTGGAAGCTTAGGTTTGTTCTCAAAAGAACAGGTGATAGAGATTG CTGTTGGTTTAGAGAAAAGTGGGCAGAGATTCTTGTGGGTGGTCCG TAATCCACCCGAGTTAGAAAAGACAGAACTGGATTTGAAATCACTC TTACCAGAAGGATTCTTAAGCCGAACCGAAGACAAAGGGATGGTCG TGAAATCATGGGCTCCGCAAGTTCCGGTTCTGAATCATAAAGCAGT CGGGGGATTCGTCACTCATTGCGGTTGGAATTCAATTCTTGAAGCT GTTTGTGCTGGTGTGCCGATGGTGGCTTGGCCGTTGTACGCTGAGC AGAGGTTTAATAGAGTGATGATTGTGGATGAGATCAAGATTGCGAT TTCGATGAATGAATCAGAGACGGGTTTCGTGAGCTCTACAGAGGTG GAGAAACGAGTCCAAGAGATAATTGGGGAGTGTCCGGTTAGGGAGC GAACCATGGCTATGAAGAACGCAGCCGAATTAGCCTTGACAGAAAC TGGTTCGTCTCATACCGCATTAACTACTTTACTCCAGTCGTGGAGC CCAAAGTGA | 5 |
| Arabidopsis thaliana | AtUGT88A1 | MGEEAIVLYPAPPIGHLVSMVELGKTILSKNPSLSIHIILVPPPYQ PESTATYISSVSSSFPSITFHHLPAVTPYSSSSTSRHHHESLLLEI LCFSNPSVHRTLFSLSRNFNVRAMIIDFFCTAVLDITADFTFPVYF FYTSGAACLAFSFYLPTIDETTPGKNLKDIPTVHIPGVPPMKGSDM PKAVLERDDEVYDVFIMFGKQLSKSSGIIINTFDALENRAIKAITE ELCFRNIYPIGPLIVNGRIEDRNDNKAVSCLNWLDSQPEKSVVFLC FGSLGLFSKEQVIEIAVGLEKSGQRFLWVVRNPPELEKTELDLKSL LPEGFLSRTEDKGMVVKSWAPQVPVLNHKAVGGFVTHCGWNSILEA VCAGVPMVAWPLYAEQRFNRVMIVDEIKIAISMNESETGFVSSTEV EKRVQEIIGECPVRERTMAMKNAAELALTETGSSHTALTTLLQSWS PK | 6 |
| Arabidopsis thaliana | AtUGT71D1 | ATGCGGAATGTAGAGCTCATCTTCATCCCCACACCAACCGTTGGTC ATCTTGTTCCGTTTCTTGAATTTGCTAGGCGTCTCATTGAGCAAGA TGATAGGATCCGTATCACAATCCTCTTGATGAAACTACAAGGTCAG TCTCATCTAGACACTTATGTTAAATCAATTGCCTCCTCTCAACCGT TTGTTAGATTCATTGATGTCCCTGAGTTAGAGGAGAAACCTACACT TGGTAGTACACAATCTGTGGAAGCTTATGTGTATGATGTTATTGAG AGAAATATCCCTCTTGTGAGGAATATAGTCATGGATATTTTAACTT CTCTTGCATTGGATGGAGTTAAGGTCAAGGGATTAGTTGTTGACTT TTTCTGTCTCCCTATGATTGACGTTGCTAAAGATATAAGTCTCCCT TTCTATGTGTTCTTGACTACAAATTCCGGGTTCTTAGCTATGATGC AGTATCTAGCAGATCGACATAGTAGAGATACATCGGTTTTTGTAAG AAACTCGGAAGAAATGTTGTCGATACCTGGATTTGTAAACCCTGTC CCAGCCAATGTTCTGCCGTCAGCTCTGTTTGTTGAAGATGGTTATG ATGCTTACGTTAAGCTGGCCATATTGTTTACAAAGGCCAATGGAAT CCTAGTGAATAGCTCCTTTGATATTGAGCCTTACTCTGTGAATCAT TTTCTTCAAGAACAGAATTATCCTTCTGTTTATGCTGTTGGCCCCA TATTTGACTTGAAAGCCCAGCCTCATCCAGAGCAGGACCTAACCCG TCGTGACGAGTTGATGAAATGGCTTGATGATCAACCCGAGGCATCG GTTGTATTCCTTTGTTTTGGGAGTATGGCAAGGTTAAGAGGTTCTC | 7 |
| | | TAGTGAAGGAAATAGCTCATGGACTTGAGCTATGTCAATATAGATT CCTCTGGTCACTCCGTAAAGAAGAGGTGACAAAGGATGATTTGCCA GAGGGGTTCCTTGACCGTGTCGATGGACGTGGAATGATATGTGGTT GGTCTCCTCAGGTAGAAATACTGGCCCATAAGGCAGTGGGAGGCTT TGTTTCTCACTGTGGATGGAACTCAATAGTAGAGAGTTTGTGGTTT GGCGTGCCAATTGTGACATGGCCAATGTATGCAGAGCAACAACTCA ATGCGTTTCTGATGGTGAAGGAACTGAAGCTAGCTGTGGAGCTGAA GCTTGATTACAGGGTACATAGTGATGAGATAGTAAACGCAAACGAG ATAGAGACCGCTATTCGTTATGTAATGGACACGGATAATAATGTTG TGAGGAAACGAGTGATGGATATCTCGCAGATGATCCAGAGAGCTAC GAAGAATGGTGGATCTTCGTTTGCCGCAATTGAGAAATTCATATAT GACGTGATAGGAATTAAGCCCTAG | |
| Arabidopsis thaliana | AtUGT71D1 | MRNVELIFIPTPTVGHLVPFLEFARRLIEQDDRIRITILLMKLQGQ SHLDTYVKSIASSQPFVRFIDVPELEEKPTLGSTQSVEAYVYDVIE RNIPLVRNIVMDILTSLALDGVKVKGLVVDFFCLPMIDVAKDISLP FYVFLTTNSGFLAMMQYLADRHSRDTSVFVRNSEEMLSIPGFVNPV PANVLPSALFVEDGYDAYVKLAILFTKANGILVNSSFDIEPYSVNH FLQEQNYPSVYAVGPIFDLKAQPHPEQDLTRRDELMKWLDDQPEAS VVFLCFGSMARLRGSLVKEIAHGLELCQYRFLWSLRKEEVTKDDLP EGFLDRVDGRGMICGWSPQVEILAHKAVGGFVSHCGWNSIVESLWF GVPIVTWPMYAEQQLNAFLMVKELKLAVELKLDYRVHSDEIVNANE IETAIRYVMDTDNNVVRKRVMDISQMIQRATKNGGSSFAAIEKFIY DVIGIKP | 8 |
| Arabidopsis thaliana | AtUGT73B4 | ATGAACAGAGAGCAAATTCATATTTTGTTCTTCCCCTTCATGGCTC ATGGCCACATGATTCCACTCTTAGACATGGCCAAGCTTTTCGCTAG AAGAGGAGCCAAATCAACTCTCCTCACAACCCCAATAAATGCTAAG ATCTTGGAGAAACCCATTGAAGCATTCAAAGTTCAAAATCCTGATC TCGAAATCGGAATCAAGATCCTCAATTTCCCTTGTGTAGAGCTTGG ATTGCCAGAAGGATGCGAGAACCGTGACTTCATTAACTCATACCAA AAATCTGACTCATTTGACTTGTTCTTGAAGTTTCTTTTCTCTACCA AGTATATGAAACAGCAGTTGGAGAGTTTCATTGAAACAACCAAACC GAGTGCTCTTGTAGCCGATATGTTCTTCCCTTGGGCAACAGAATCC GCGGAGAAGATCGGTGTTCCAAGACTTGTGTTCCACGGCACATCAT CCTTTGCCTTGTGTTGTTCGTATAACATGAGGATTCATAAGCCACA CAAGAAAGTCGCTTCGAGTTCTACTCCATTTGTAATCCCTGGTCTC CCTGGAGACATAGTTATTACAGAAGACCAAGCCAATGTCACCAACG AAGAAACTCCATTCGGAAAGTTTTGGAAAGAAGTCAGGGAATCAGA GACCAGTAGCTTTGGTGTTTTGGTGAATAGCTTCTACGAGCTGGAA TCATCTTATGCTGATTTTTACCGTAGTTTTGTGGCGAAAAAAGCGT GGCATATAGGTCCACTTTCACTATCCAACAGAGGGATTGCAGAGAA AGCCGGAAGAGGGAAAAAGGCAAACATTGATGAGCAAGAATGCCTC AAATGGCTTGACTCTAAGACACCTGGCTCAGTAGTTTACTTGTCCT TTGGTAGCGGAACCGGCTTACCCAACGAACAGCTGTTAGAGATTGC TTTCGGCCTTGAAGGCTCTGGACAAAATTTCATTTGGGTGGTTAGC AAAAATGAAAACCAAGTTGGGACAGGTGAAAATGAAGATTGGTTGC CTAAAGGGTTTGAAGAGAGGAATAAAGGAAAAGGGCTGATAATACG CGGATGGGCCCCGCAAGTGCTGATACTTGACCACAAAGCAATCGGA GGATTTGTGACGCATTGCGGATGGAACTCGACTTTGGAGGGCATTG CCGCAGGGCTGCCTATGGTGACTTGGCCGATGGGGGCAGAACAGTT CTACAACGAGAAGTTATTGACAAAAGTGTTGAGAATAGGAGTGAAC GTTGGAGCTACCGAGTTGGTGAAAAAAGGAAAGTTGATTAGTAGAG CACAAGTGGAGAAGGCAGTAAGGGAAGTGATTGGTGGTGAGAAGGC AGAGGAAAGGCGGCTAAGGGCTAAGGAGCTGGGCGAGATGGCTAAA GCCGCTGTGGAAGAAGGAGGGTCTTCTTATAATGATGTGAACAAGT TTATGGAAGAGCTGAATGGTAGAAAGTAG | 9 |
| Arabidopsis thaliana | AtUGT73B4 | MNREQIHILFFPFMAHGHMIPLLDMAKLFARRGAKSTLLTTPINAK ILEKPIEAFKVQNPDLEIGIKILNFPCVELGLPEGCENRDFINSYQ KSDSFDLFLKFLFSTKYMKQQLESFIETTKPSALVADMFFPWATES AEKIGVPRLVFHGTSSFALCCSYNMRIHKPHKKVASSSTPFVIPGL PGDIVITEDQANVTNEETPFGKFWKEVRESETSSFGVLVNSFYELE SSYADFYRSFVAKKAWHIGPLSLSNRGIAEKAGRGKKANIDEQECL KWLDSKTPGSVVYLSFGSGTGLPNEQLLEIAFGLEGSGQNFIWVVS KNENQVGTGENEDWLPKGFEERNKGKGLIIRGWAPQVLILDHKAIG | 10 |
| | | GFVTHCGWNSTLEGIAAGLPMVTWPMGAEQFYNEKLLTKVLRIGVN VGATELVKKGKLISRAQVEKAVREVIGGEKAEERRLRAKELGEMAK AAVEEGGSSYNDVNKFMEELNGRK | |
| Arabidopsis thaliana | AtUGT76C4 | ATGGAGAAGAGTAATGGCCTGCGAGTGATTCTGTTTCCACTTCCAT TACAAGGCTGCATCAACCCTATGATTCAGCTCGCCAAGATCCTCCA CTCAAGAGGTTTTTCAATCACTGTGATCCACACTTGCTTCAACGCG CCAAAAGCTTCAAGCCATCCACTCTTCACCTTCATACAGATCCAAG ATGGCTTGTCTGAAACAGAGACAAGAACTCGCGACGTCAAACTTCT CATAACACTTCTCAACCAAAATTGCGAGTCTCCGGTTCGTGAATGT TTGCGTAAACTGTTGCAATCTGCCAAGGAAGAGAAACAGAGGATTA GCTGTTTGATCAATGATTCTGGTTGGATCTTCACTCAACACTTAGC CAAGAGTTTGAATCTCATGAGATTGGCCTTTAATACCTATAAGATC TCCTTCTTTCGAAGCCATTTTGTTCTTCCTCAGCTCCGGCGTGAAA TGTTTCTTCCATTACAAGATTCAGAACAAGATGATCCAGTTGAGAA GTTTCCACCGCTTAGAAAGAAAGATCTTTTACGGATTCTTGAAGCA GATTCGGTGCAGGGAGACTCGTACTCGGATATGATTTTGGAAAAGA CAAAGGCGTCTTCAGGTCTTATATTCATGTCCTGTGAAGAGTTGGA CCAAGACTCACTGAGTCAATCACGTGAAGATTTTAAGGTTCCGATA TTTGCGATAGGACCTTCTCATAGCCATTTTCCTGCTTCTTCTAGTA GCTTGTTCACACCGGACGAGACTTGCATCCCATGGTTAGACAGACA AGAAGACAAATCCGTAATATACGTGAGTATTGGGAGCCTCGTGACC ATCAACGAAACAGAGCTAATGGAGATTGCTTGGGGTCTAAGTAACA GCGACCAACCATTTTTATGGGTCGTCCGGGTTGGTTCAGTCAATGG CACGGAATGGATTGAAGCAATCCCGGAATATTTCATCAAAAGGCTT AATGAGAAGGGAAAGATAGTGAAATGGGCTCCACAACAAGAGGTTC TAAAGCATCGAGCTATTGGAGGTTTCTTGACACATAATGGTTGGAA CTCGACGGTTGAGAGTGTTTGTGAAGGCGTCCCTATGATCTGTTTG CCTTTTCGTTGGGACCAATTGTTAAATGCAAGATTTGTTAGTGATG TATGGATGGTTGGGATACATCTCGAGGGTCGGATTGAGAGGGATGA GATCGAGAGAGCGATAAGGAGATTATTGTTGGAAACTGAAGGAGAA GCCATCCGAGAGAGGATACAACTTCTTAAGGAAAAAGTAGGAAGAT CAGTTAAACAAAACGGTTCGGCATATCAATCTCTACAAAATTTGAT TAATTATATATCATCTTTCTAG | 11 |
| Arabidopsis thaliana | AtUGT76C4 | MEKSNGLRVILFPLPLQGCINPMIQLAKILHSRGFSITVIHTCFNA PKASSHPLFTFIQIQDGLSETETRTRDVKLLITLLNQNCESPVREC LRKLLQSAKEEKQRISCLINDSGWIFTQHLAKSLNLMRLAFNTYKI SFFRSHFVLPQLRREMFLPLQDSEQDDPVEKFPPLRKKDLLRILEA DSVQGDSYSDMILEKTKASSGLIFMSCEELDQDSLSQSREDFKVPI FAIGPSHSHFPASSSSLFTPDETCIPWLDRQEDKSVIYVSIGSLVT INETELMEIAWGLSNSDQPFLWVVRVGSVNGTEWIEAIPEYFIKRL NEKGKIVKWAPQQEVLKHRAIGGFLTHNGWNSTVESVCEGVPMICL PFRWDQLLNARFVSDVWMVGIHLEGRIERDEIERAIRRLLLETEGE AIRERIQLLKEKVGRSVKQNGSAYQSLQNLINYISSF | 12 |
| Arabidopsis thaliana | AtUGT76E1 2 | ATGCAGGTTTTGGGAATGGAGGAAAAGCCTGCAAGGAGAAGCGTAG TGTTGGTTCCATTTCCAGCACAAGGACATATATCTCCAATGATGCA ACTTGCCAAAACCCTTCACTTAAAGGGTTTCTCGATCACAGTTGTT CAGACTAAGTTCAATTACTTTAGCCCTTCAGATGACTTCACTCATG ATTTTCAGTTCGTCACCATTCCAGAAAGCTTACCAGAGTCTGATTT CAAGAATCTCGGACCAATACAGTTTCTGTTTAAGCTCAACAAAGAG TGTAAGGTGAGCTTCAAGGACTGTTTGGGTCAGTTGGTGCTGCAAC AAAGTAATGAGATCTCATGTGTCATCTACGATGAGTTCATGTACTT TGCTGAAGCTGCAGCCAAAGAGTGTAAGCTTCCAAACATCATTTTC AGCACAACAAGTGCCACGGCTTTCGCTTGCCGCTCTGTATTTGACA AACTATATGCAAACAATGTCCAAGCTCCCTTGAAAGAAACTAAAGG ACAACAAGAAGAGCTAGTTCCGGAGTTTTATCCCTTGAGATATAAA GACTTTCCAGTTTCACGGTTTGCATCATTAGAGAGCATAATGGAGG TGTATAGGAATACAGTTGACAAACGGACAGCTTCCTCGGTGATAAT CAACACTGCGAGCTGTCTAGAGAGCTCATCTCTGTCTTTTCTGCAA CAACAACAGCTACAAATTCCAGTGTATCCTATAGGCCCTCTTCACA TGGTGGCCTCAGCTCCTACAAGTCTGCTTGAAGAGAACAAGAGCTG CATCGAATGGTTGAACAAACAAAAGGTAAACTCGGTGATATACATA AGCATGGGAAGCATAGCTTTAATGGAAATCAACGAGATAATGGAAG TCGCGTCAGGATTGGCTGCTAGCAACCAACACTTCTTATGGGTGAT | 13 |
| | | CCGACCAGGGTCAATACCTGGTTCCGAGTGGATAGAGTCCATGCCT GAAGAGTTTAGTAAGATGGTTTTGGACCGAGGTTACATTGTGAAAT GGGCTCCACAGAAGGAAGTACTTTCTCATCCTGCAGTAGGAGGGTT TTGGAGCCATTGTGGATGGAACTCGACACTAGAAAGCATCGGCCAA GGAGTTCCAATGATCTGCAGGCCATTTTCGGGTGATCAAAAGGTGA ACGCTAGATACTTGGAGTGTGTATGGAAAATTGGGATTCAAGTGGA GGGTGAGCTAGACAGAGGAGTGGTCGAGAGAGCTGTGAAGAGGTTA ATGGTTGACGAAGAAGGAGAGGAGATGAGGAAGAGAGCTTTCAGTT TAAAAGAGCAACTTAGAGCCTCTGTTAAAAGTGGAGGCTCTTCACA CAACTCGCTAGAAGAGTTTGTACACTTCATAAGGACTCTATGA | |
| Arabidopsis thaliana | AtUGT76E1 2 | MQVLGMEEKPARRSVVLVPFPAQGHISPMMQLAKTLHLKGFSITVV QTKFNYFSPSDDFTHDFQFVTIPESLPESDFKNLGPIQFLFKLNKE CKVSFKDCLGQLVLQQSNEISCVIYDEFMYFAEAAAKECKLPNIIF STTSATAFACRSVFDKLYANNVQAPLKETKGQQEELVPEFYPLRYK DFPVSRFASLESIMEVYRNTVDKRTASSVIINTASCLESSSLSFLQ QQQLQIPVYPIGPLHMVASAPTSLLEENKSCIEWLNKQKVNSVIYI SMGSIALMEINEIMEVASGLAASNQHFLWVIRPGSIPGSEWIESMP EEFSKMVLDRGYIVKWAPQKEVLSHPAVGGFWSHCGWNSTLESIGQ GVPMICRPFSGDQKVNARYLECVWKIGIQVEGELDRGVVERAVKRL MVDEEGEEMRKRAFSLKEQLRASVKSGGSSHNSLEEFVHFIRTL | 14 |
| Cannabis sativa | CsUGT73C 6 | ATGGCCTCTGAACCATACAAATTGCATTTGATCGTTATCCCATTCA TGGCTCCAGGTCATTTTATTCCAATGGCTGATATGGCTAGAAAGTT GGCTGAACATGGTGCTATGATTACTTTGATTACCTTGCCAGTTATT GCCGCCAGAATTAGACCAATTATTGAACAAGCTACCGAGAACTCCA ACTTGAAGATTCAATTGGTTCAAGTCTCCTTGCCATTGCATGAATT TGGTTTGCCTGAAGGTTGTGATACCGTTGATTTGGTTCCATCTAGA AACCTGTTGCTGTCTTTCTTCATTGCCTTGGATGAATTGCAACAGC CAATCGAACAAGTTGTCTCTGAATTGAAACCTAGACCATCCTGTAT TATTGCCGACAAACATTTGCCATGGACTGCTGAAATTGCTACCAAG TTTGGTATTCCAAGAGTTTTGTTCGATGGTATGTCTTGCTTCTCTT TGTTGTGCAACCATATGATCAGAAAGTCCCAAGTTCATTTGTCCGT TCCAATGTCTGTTCCATTTGTTGTTCCAGGTATGCCAGATCATTTG GAGTTCACTAGAAATCAATTGGCTGCTGACTTGTACCCTAATTTGG AATTTGGTCAAAAGTTCCACGACAGGATCAGAGAATCTGAAGAAGG TGCTGACGGTTTTTTGGTTAACTCTTTCGAAGAATTGGAGTGGAAG TACGTTGAGGGTTACAGAAAAGAAAAAGTTGGTAAGGCTTGGTGCA TTGGTCCAGTTTCTTTGTTTAACAAGACCAAGTTGGAAGTTGCCCA AAGAGGTAACAATCCAGCTGGTGCTGTTGACGAAAAACAATGTACT GAATGGTTGGATTCTTGGCCAAAGTCTTGTGTTGTTTATGCTTGTT TGGGTTCCGTTTCCAGATTATTGATTCCACAGATGATCGAATTGGG TGTTGCTTTGGAAGCTTCTAACAAACCATTCATCTGGGTTATCAGA GGTTACGATCAAGAGGAAGAAATCGAAAAGTGGATCTCCGAATCTG GTTTTAAAGAAAGGACTAAGTCCAGAGCCTTGTTGATTTTTGGTTG GGCTCCACAAGTTCTGATTTTGTCTCATTCTTCTGTTGGTGGTTTC TTGACTCATTGTGGTTGGAATTCTACCTTGGAAGGTATTACTTATG GCAAGCCAATGATTACATGGCCAATGTTCGCTGAACAATTCTACAA TCAAAAGTTGATCGTCCAGGTTTTGAAGGTTGGTGAATCTGTTGGT CCAAAATTCGTTGTTCCATATGGTAGAGAAGAGGAATTCGGTGTTT TCGTTTTGTCCAAGGATATTTTGGAAGCCATCGAAAAGGTTATGGC CCAAGACAAAGAAGGTGAAGAACGTAGAGAAAGAGTCAAGAGATTG TCAGATATGGCTCAAAAGGCTATTGAGGAAGGTGGTTCTTCTTACT TGGATATGAAGTTGTTCATCGAGGACATCAGAAACTTGCACATCTC TTGA | 15 |
| Cannabis sativa | CsUGT73C 6 | MASEPYKLHLIVIPFMAPGHFIPMADMARKLAEHGAMITLITLPVI AARIRPIIEQATENSNLKIQLVQVSLPLHEFGLPEGCDTVDLVPSR NLLLSFFIALDELQQPIEQVVSELKPRPSCIIADKHLPWTAEIATK FGIPRVLFDGMSCFSLLCNHMIRKSQVHLSVPMSVPFVVPGMPDHL EFTRNQLAADLYPNLEFGQKFHDRIRESEEGADGFLVNSFEELEWK YVEGYRKEKVGKAWCIGPVSLFNKTKLEVAQRGNNPAGAVDEKQCT EWLDSWPKSCVVYACLGSVSRLLIPQMIELGVALEASNKPFIWVIR GYDQEEEIEKWISESGFKERTKSRALLIFGWAPQVLILSHSSVGGF LTHCGWNSTLEGITYGKPMITWPMFAEQFYNQKLIVQVLKVGESVG | 16 |
| | | PKFVVPYGREEEFGVFVLSKDILEAIEKVMAQDKEGEERRERVKRL SDMAQKAIEEGGSSYLDMKLFIEDIRNLHIS | |
| Arabidopsis thaliana | At5g49690 | ATGGTCGACAAGAGAGAAGAAGTTATGCACGTAGCCATGTTTCCAT GGCTAGCTATGGGTCATCTCCTTCCTTTTCTTCGTCTCTCCAAGTT ACTAGCTCAAAAGGGTCACAAGATCTCTTTCATATCAACACCAAGA AACATCGAAAGACTTCCTAAATTACAATCAAACCTCGCCTCCTCCA TCACCTTCGTCTCTTTCCCTCTCCCTCCCATCTCAGGCTTGCCTCC TTCTTCAGAATCATCCATGGACGTTCCTTACAACAAGCAACAGTCT CTTAAAGCCGCTTTTGATCTTCTTCAGCCACCGTTGAAAGAGTTTC TCCGACGGTCTTCTCCGGATTGGATCATATACGACTATGCTTCTCA CTGGCTTCCTTCTATTGCGGCCGAGCTTGGAATCTCTAAGGCTTTC TTTAGTCTCTTTAACGCAGCTACTCTCTGTTTCATGGGACCGTCTT CGTCTTTGATTGAAGAAATTAGATCAACGCCGGAAGATTTCACGGT GGTGCCACCGTGGGTCCCGTTCAAGTCAAACATCGTGTTTCGTTAT CATGAAGTTACTAGATACGTTGAGAAGACAGAGGAAGATGTAACCG GAGTCTCTGACTCAGTTCGGTTTGGTTACTCGATTGACGAAAGCGA TGCGGTTTTTGTCCGTAGCTGTCCGGAGTTTGAACCGGAATGGTTT GGTTTACTAAAAGACCTGTACCGTAAACCGGTATTTCCAATCGGGT TTTTGCCTCCGGTTATTGAAGACGACGATGCCGTTGATACTACATG GGTTCGTATAAAGAAGTGGCTCGACAAGCAACGGCTTAATTCAGTT GTTTACGTGTCACTTGGCACCGAAGCGAGTCTTCGTCATGAGGAAG TAACTGAGCTAGCTCTTGGGTTAGAGAAGTCAGAGACACCGTTCTT TTGGGTCCTAAGGAACGAGCCAAAGATTCCAGATGGGTTCAAAACA CGAGTCAAGGGACGTGGAATGGTTCATGTTGGTTGGGTTCCACAAG TGAAAATACTTAGTCACGAGTCAGTAGGAGGGTTCTTGACACATTG TGGTTGGAACTCAGTGGTGGAAGGGTTAGGGTTTGGTAAAGTTCCA ATCTTTTTTCCGGTGTTGAATGAGCAAGGACTTAATACGAGGTTGT TGCATGGGAAAGGACTTGGTGTTGAGGTTTCAAGAGATGAGAGAGA TGGGTCGTTTGATTCTGACTCGGTCGCTGACTCGATTAGGTTGGTG ATGATTGATGATGCTGGCGAGGAGATAAGGGCTAAGGCTAAAGTGA TGAAGGATTTGTTTGGGAACATGGATGAGAATATTCGTTATGTTGA CGAACTTGTTAGGTTTATGAGAAGTAAAGGATCATCATCATCATCA TGA | 17 |
| Arabidopsis thaliana | At5g49690 | MVDKREEVMHVAMFPWLAMGHLLPFLRLSKLLAQKGHKISFISTPR NIERLPKLQSNLASSITFVSFPLPPISGLPPSSESSMDVPYNKQQS LKAAFDLLQPPLKEFLRRSSPDWIIYDYASHWLPSIAAELGISKAF FSLFNAATLCFMGPSSSLIEEIRSTPEDFTVVPPWVPFKSNIVFRY HEVTRYVEKTEEDVTGVSDSVRFGYSIDESDAVFVRSCPEFEPEWF GLLKDLYRKPVFPIGFLPPVIEDDDAVDTTWVRIKKWLDKQRLNSV VYVSLGTEASLRHEEVTELALGLEKSETPFFWVLRNEPKIPDGFKT RVKGRGMVHVGWVPQVKILSHESVGGFLTHCGWNSVVEGLGFGKVP IFFPVLNEQGLNTRLLHGKGLGVEVSRDERDGSFDSDSVADSIRLV MIDDAGEEIRAKAKVMKDLFGNMDENIRYVDELVRFMRSKGSSSSS | 18 |
| Stevia rebaudiana | SrUGT76G1 | ATGGAAAATAAAACGGAGACCACCGTTCGCCGGCGCCGGAGAATAA TATTATTCCCGGTACCATTTCAAGGCCACATTAACCCAATTCTTCA GCTAGCCAATGTGTTGTACTCTAAAGGATTCAGTATCACCATCTTT CACACCAACTTCAACAAACCCAAAACATCTAATTACCCTCACTTCA CTTTCAGATTCATCCTCGACAACGACCCACAAGACGAACGCATTTC CAATCTACCGACTCATGGTCCGCTCGCTGGTATGCGGATTCCGATT ATCAACGAACACGGAGCTGACGAATTACGACGCGAACTGGAACTGT TGATGTTAGCTTCTGAAGAAGATGAAGAGGTATCGTGTTTAATCAC GGATGCTCTTTGGTACTTCGCGCAATCTGTTGCTGACAGTCTTAAC CTCCGACGGCTTGTTTTGATGACAAGCAGCTTGTTTAATTTTCATG CACATGTTTCACTTCCTCAGTTTGATGAGCTTGGTTACCTCGATCC TGATGACAAAACCCGTTTGGAAGAACAAGCGAGTGGGTTTCCTATG CTAAAAGTGAAAGACATCAAGTCTGCGTATTCGAACTGGCAAATAC TCAAAGAGATATTAGGGAAGATGATAAAACAAACAAGAGCATCTTC AGGAGTCATCTGGAACTCATTTAAGGAACTCGAAGAGTCTGAGCTC GAAACTGTTATCCGTGAGATCCCGGCTCCAAGTTTCTTGATACCAC TCCCCAAGCATTTGACAGCCTCTTCCAGCAGCTTACTAGACCACGA TCGAACCGTTTTTCAATGGTTAGACCAACAACCGCCAAGTTCGGTA CTGTATGTTAGTTTTGGTAGTACTAGTGAAGTGGATGAGAAAGATT TCTTGGAAATAGCTCGTGGGTTGGTTGATAGCAAGCAGTCGTTTTT | 19 |
| | | ATGGGTGGTTCGACCTGGGTTTGTCAAGGGTTCGACGTGGGTCGAA CCGTTGCCAGATGGGTTCTTGGGTGAAAGAGGACGTATTGTGAAAT GGGTTCCACAGCAAGAAGTGCTAGCTCATGGAGCAATAGGCGCATT CTGGACTCATAGCGGATGGAACTCTACGTTGGAAAGCGTTTGTGAA GGTGTTCCTATGATTTTCTCGGATTTTGGGCTCGATCAACCGTTGA ATGCTAGATACATGAGTGATGTTTTGAAGGTAGGGGTGTATTTGGA AAATGGGTGGGAAAGAGGAGAGATAGCAAATGCAATAAGAAGAGTT ATGGTGGATGAAGAAGGAGAATACATTAGACAGAATGCAAGAGTTT TGAAACAAAAGGCAGATGTTTCTTTGATGAAGGGTGGTTCGTCTTA CGAATCATTAGAGTCTCTAGTTTCTTACATTTCATCGTTGTAA | |
| Stevia rebaudiana | SrUGT76G1 | MENKTETTVRRRRRIILFPVPFQGHINPILQLANVLYSKGFSITIF HTNFNKPKTSNYPHFTFRFILDNDPQDERISNLPTHGPLAGMRIPI INEHGADELRRELELLMLASEEDEEVSCLITDALWYFAQSVADSLN LRRLVLMTSSLFNFHAHVSLPQFDELGYLDPDDKTRLEEQASGFPM LKVKDIKSAYSNWQILKEILGKMIKQTRASSGVIWNSFKELEESEL ETVIREIPAPSFLIPLPKHLTASSSSLLDHDRTVFQWLDQQPPSSV LYVSFGSTSEVDEKDFLEIARGLVDSKQSFLWVVRPGFVKGSTWVE PLPDGFLGERGRIVKWVPQQEVLAHGAIGAFWTHSGWNSTLESVCE GVPMIFSDFGLDQPLNARYMSDVLKVGVYLENGWERGEIANAIRRV MVDEEGEYIRQNARVLKQKADVSLMKGGSSYESLESLVSYISSL | 20 |
| Arabidopsis thaliana | AtUGT85A3 | ATGGGATCCCGTTTTGTTTCTAACGAACAAAAACCACACGTAGTTT GCGTGCCTTACCCAGCTCAAGGCCACATTAACCCTATGATGAAAGT GGCTAAACTCCTCCACGTCAAAGGCTTCCACGTCACCTTCGTCAAC ACCGTCTACAACCACAACCGTCTACTCCGATCCCGTGGGGCCAACG CACTCGATGGACTTCCTTCCTTCCAGTTCGAGTCAATACCTGACGG TCTTCCGGAGACTGGCGTGGACGCCACGCAGGACATCCCTGCCCTT TCCGAGTCCACAACGAAAAACTGTCTCGTTCCGTTCAAGAAGCTTC TCCAGCGGATTGTCACGAGAGAGGATGTCCCTCCGGTGAGCTGTAT TGTATCAGATGGTTCGATGAGCTTTACTCTTGACGTAGCGGAAGAG CTTGGTGTTCCGGAGATTCATTTTTGGACCACTAGTGCTTGTGGCT TCATGGCTTATCTACACTTTTATCTCTTCATCGAGAAGGGTTTATG TCCAGTAAAAGATGCGAGTTGCTTGACGAAGGAATACTTGGACACA GTTATAGATTGGATACCGTCAATGAACAATGTAAAACTAAAAGACA TTCCTAGTTTTATACGTACCACTAATCCTAACGACATAATGCTCAA CTTCGTTGTCCGTGAGGCATGTCGAACCAAACGTGCCTCTGCTATC ATTCTGAACACGTTTGATGACCTTGAACATGACATAATCCAGTCTA TGCAATCCATTTTACCACCGGTTTATCCAATCGGACCGCTTCATCT CTTAGTAAACAGGGAGATTGAAGAAGATAGTGAGATTGGAAGGATG GGATCAAATCTATGGAAAGAGGAGACTGAGTGCTTGGGATGGCTTA ATACTAAGTCTCGAAATAGCGTTGTTTATGTTAACTTTGGGAGCAT AACAATAATGACCACGGCACAGCTTTTGGAGTTTGCTTGGGGTTTG GCGGCAACGGGAAAGGAGTTTCTATGGGTGATGCGGCCGGATTCAG TAGCCGGAGAGGAGGCAGTGATTCCAAAAGAGTTTTTAGCGGAGAC AGCTGATCGAAGAATGCTGACAAGTTGGTGTCCTCAGGAGAAAGTT CTTTCTCATCCGGCGGTCGGAGGGTTCTTGACCCATTGCGGGTGGA ATTCGACGTTAGAAAGTCTTTCATGCGGAGTTCCAATGGTATGTTG GCCATTTTTTGCTGAGCAACAAACAAATTGTAAGTTTTCTTGTGAT GAATGGGAGGTTGGTATTGAGATCGGTGGAGATGTCAAGAGGGGAG AGGTTGAGGCGGTGGTTAGAGAGCTCATGGATGGAGAGAAAGGAAA GAAAATGAGAGAGAAGGCTGTAGAGTGGCGGCGCTTGGCCGAGAAA GCTACAAAGCTTCCGTGTGGTTCGTCGGTGATAAATTTTGAGACGA TTGTCAACAAGGTTCTCTTGGGAAAGATCCCTAACACGTAA | 21 |
| Arabidopsis thaliana | AtUGT85A3 | MGSRFVSNEQKPHVVCVPYPAQGHINPMMKVAKLLHVKGFHVTFVN TVYNHNRLLRSRGANALDGLPSFQFESIPDGLPETGVDATQDIPAL SESTTKNCLVPFKKLLQRIVTREDVPPVSCIVSDGSMSFTLDVAEE LGVPEIHFWTTSACGFMAYLHFYLFIEKGLCPVKDASCLTKEYLDT VIDWIPSMNNVKLKDIPSFIRTTNPNDIMLNFVVREACRTKRASAI ILNTFDDLEHDIIQSMQSILPPVYPIGPLHLLVNREIEEDSEIGRM GSNLWKEETECLGWLNTKSRNSVVYVNFGSITIMTTAQLLEFAWGL AATGKEFLWVMRPDSVAGEEAVIPKEFLAETADRRMLTSWCPQEKV LSHPAVGGFLTHCGWNSTLESLSCGVPMVCWPFFAEQQTNCKFSCD EWEVGIEIGGDVKRGEVEAVVRELMDGEKGKKMREKAVEWRRLAEK ATKLPCGSSVINFETIVNKVLLGKIPNT | 22 |
| Arabidopsis thaliana | AtUGT73B1 | ATGGGTGTTTTTGGATCGAATGAATCGTCAAGCATGAGTATTGTGA TGTATCCGTGGTTAGCCTTTGGTCACATGACTCCTTTTCTTCACCT ATCCAACAAGCTCGCAGAGAAAGGTCACAAGATTGTTTTCTTGCTT CCCAAGAAAGCACTAAACCAGCTTGAACCTCTTAATCTCTACCCAA ATCTCATCACTTTCCACACCATCTCTATCCCTCAGGTCAAAGGGCT CCCTCCGGGTGCGGAGACAAACTCCGACGTCCCTTTCTTCTTGACA CATTTGCTTGCAGTTGCAATGGACCAAACCCGGCCAGAGGTCGAGA CCATTTTCCGTACAATCAAACCGGACTTGGTTTTCTATGATTCTGC CCATTGGATACCGGAAATTGCTAAACCGATCGGTGCTAAAACCGTT TGCTTCAACATCGTTAGCGCTGCGTCAATCGCACTGTCTCTTGTCC CTTCTGCGGAGAGAGAGGTCATTGATGGCAAGGAAATGTCAGGGGA GGAGTTAGCTAAGACGCCTCTAGGTTACCCATCTTCGAAAGTAGTC TTACGTCCGCACGAAGCAAAATCCCTGAGTTTCGTGTGGAGGAAGC ACGAGGCGATTGGCTCTTTCTTTGATGGGAAAGTTACCGCGATGAG AAACTGCGACGCAATCGCTATAAGGACTTGCCGTGAGACAGAAGGC AAATTCTGCGATTACATAAGTAGGCAGTACAGTAAACCGGTTTACC TAACAGGACCGGTTCTCCCTGGATCCCAACCTAATCAGCCCTCCTT AGATCCTCAATGGGCGGAGTGGCTAGCCAAATTCAACCACGGTTCG GTTGTGTTCTGCGCTTTCGGTAGCCAACCCGTTGTAAACAAGATAG ATCAGTTTCAAGAACTCTGTTTAGGTCTAGAATCAACTGGTTTTCC GTTTCTGGTTGCCATTAAGCCTCCTTCGGGTGTATCAACCGTCGAG GAAGCCTTACCGGAAGGATTCAAAGAGAGGGTTCAAGGACGTGGCG TTGTGTTTGGAGGTTGGATTCAGCAACCGTTGGTGTTGAACCATCC TTCAGTGGGTTGTTTTGTTAGCCATTGCGGGTTTGGGTCGATGTGG GAGTCGTTGATGAGTGATTGTCAGATCGTTTTGGTTCCGCAGCACG GAGAACAGATTTTGAACGCAAGGCTGATGACGGAGGAGATGGAGGT GGCGGTTGAAGTGGAGAGGGAAAAGAAAGGGTGGTTCTCGCGGCAA AGCTTGGAGAATGCTGTGAAGAGTGTGATGGAGGAAGGTAGTGAGA TCGGTGAGAAAGTGAGGAAGAATCATGACAAGTGGAGATGTGTTTT GACTGACTCTGGTTTTTCAGATGGTTATATTGATAAGTTTGAACAA AATTTAATTGAACTTGTGAAGTCATGA | 23 |
| Arabidopsis thaliana | AtUGT73B1 | MGVFGSNESSSMSIVMYPWLAFGHMTPFLHLSNKLAEKGHKIVFLL PKKALNQLEPLNLYPNLITFHTISIPQVKGLPPGAETNSDVPFFLT HLLAVAMDQTRPEVETIFRTIKPDLVFYDSAHWIPEIAKPIGAKTV CFNIVSAASIALSLVPSAEREVIDGKEMSGEELAKTPLGYPSSKVV LRPHEAKSLSFVWRKHEAIGSFFDGKVTAMRNCDAIAIRTCRETEG KFCDYISRQYSKPVYLTGPVLPGSQPNQPSLDPQWAEWLAKFNHGS VVFCAFGSQPVVNKIDQFQELCLGLESTGFPFLVAIKPPSGVSTVE EALPEGFKERVQGRGVVFGGWIQQPLVLNHPSVGCFVSHCGFGSMW ESLMSDCQIVLVPQHGEQILNARLMTEEMEVAVEVEREKKGWFSRQ SLENAVKSVMEEGSEIGEKVRKNHDKWRCVLTDSGFSDGYIDKFEQ NLIELVKS | 24 |
| Arabidopsis thaliana | At5g65550 | ATGGCCGAGCCAAAACCGAAGCTTCATGTTGCAGTGTTCCCATGGT TAGCTTTAGGTCACATGATTCCTTACTTGCAACTCTCAAAGCTCAT AGCAAGGAAAGGCCATACTGTGTCCTTCATCTCCACAGCTCGTAAC ATTTCACGTCTTCCCAATATATCCTCCGACCTTTCCGTGAATTTCG TTTCTTTGCCGTTAAGTCAAACCGTCGACCATCTCCCAGAGAACGC TGAGGCCACCACTGATGTCCCGGAGACTCACATAGCTTATCTGAAG AAAGCATTTGATGGGCTTTCTGAAGCTTTCACAGAGTTTTTAGAAG CTTCCAAACCAAACTGGATAGTGTATGATATCTTGCACCATTGGGT CCCGCCTATCGCTGAGAAGCTCGGCGTGAGACGAGCCATCTTCTGC ACGTTCAACGCAGCTTCCATCATCATCATCGGTGGGCCAGCATCAG TCATGATTCAAGGTCATGACCCTCGAAAGACTGCTGAAGATCTTAT CGTGCCTCCACCATGGGTCCCGTTTGAGACCAACATAGTTTACCGT CTCTTTGAAGCTAAGAGGATCATGGAGTATCCCACGGCAGGTGTAA CTGGAGTTGAATTGAACGACAACTGTAGATTGGGTTTGGCTTACGT TGGCTCTGAGGTTATTGTGATTAGATCATGTATGGAACTCGAACCT GAGTGGATTCAATTGCTCAGTAAACTCCAAGGAAAGCCTGTGATTC CAATTGGTTTACTCCCGGCTACACCAATGGATGATGCAGATGACGA GGGAACATGGTTAGACATCAGAGAATGGCTAGACAGACATCAAGCA AAGTCTGTGGTTTATGTAGCCTTAGGAACTGAAGTGACAATTAGTA ACGAAGAGATTCAAGGTTTAGCTCATGGGTTGGAGCTTTGCAGGTT ACCTTTCTTTTGGACGCTAAGGAAGAGGACTAGAGCTTCTATGCTA | 25 |
| | | CTACCTGATGGGTTCAAAGAGAGAGTCAAAGAGCGTGGAGTCATTT GGACCGAGTGGGTACCTCAGACCAAGATACTGAGCCATGGTTCAGT TGGTGGGTTTGTTACTCATTGTGGTTGGGGATCAGCTGTGGAAGGG CTTAGCTTTGGTGTCCCTTTGATCATGTTTCCATGTAACCTAGACC AGCCGCTAGTGGCTAGGTTGCTCAGTGGGATGAATATAGGCTTGGA GATTCCAAGGAATGAGCGAGACGGGCTGTTCACGAGTGCTTCTGTT GCAGAGACAATCAGACATGTTGTTGTGGAAGAAGAAGGAAAGATCT ACAGGAACAATGCTGCATCTCAGCAAAAGAAAATATTCGGGAACAA GAGATTGCAAGATCAGTATGCGGATGGTTTTATCGAGTTTCTGGAG AATCCTATAGCAGGAGTGTAG | |
| Arabidopsis thaliana | Atg65550 | MAEPKPKLHVAVFPWLALGHMIPYLQLSKLIARKGHTVSFISTARN ISRLPNISSDLSVNFVSLPLSQTVDHLPENAEATTDVPETHIAYLK KAFDGLSEAFTEFLEASKPNWIVYDILHHWVPPIAEKLGVRRAIFC TFNAASIIIIGGPASVMIQGHDPRKTAEDLIVPPPWVPFETNIVYR LFEAKRIMEYPTAGVTGVELNDNCRLGLAYVGSEVIVIRSCMELEP EWIQLLSKLQGKPVIPIGLLPATPMDDADDEGTWLDIREWLDRHQA KSVVYVALGTEVTISNEEIQGLAHGLELCRLPFFWTLRKRTRASML LPDGFKERVKERGVIWTEWVPQTKILSHGSVGGFVTHCGWGSAVEG LSFGVPLIMFPCNLDQPLVARLLSGMNIGLEIPRNERDGLFTSASV AETIRHVVVEEEGKIYRNNAASQQKKIFGNKRLQDQYADGFIEFLE NPIAGV | 26 |
| Arabidopsis thaliana | AtUGT76B1 | ATGGAGACTAGAGAAACAAAACCAGTGATCTTTCTCTTCCCTTTCC CTTTACAAGGTCACTTAAACCCAATGTTTCAGCTCGCCAACATCTT CTTCAACAGAGGCTTCTCCATCACTGTGATCCACACTGAGTTCAAC TCTCCAAACTCTTCCAATTTCCCTCATTTCACTTTCGTATCCATCC CCGATAGCTTGTCTGAACCTGAATCCTATCCCGATGTCATCGAGAT TCTCCATGACCTCAATTCCAAGTGTGTTGCTCCTTTTGGTGATTGC TTAAAGAAGCTTATATCTGAAGAACCAACAGCAGCTTGTGTGATTG TTGACGCTCTTTGGTACTTCACTCACGATTTAACCGAGAAATTCAA TTTCCCGAGGATTGTTCTCCGAACCGTTAACCTCTCAGCTTTCGTC GCTTTCTCAAAGTTTCATGTTTTACGAGAGAAAGGGTATCTTTCTT TACAAGAGACTAAGGCAGACTCACCGGTTCCGGAGCTTCCGTATCT TAGAATGAAGGATCTTCCATGGTTCCAGACAGAAGATCCAAGATCA GGGGATAAGTTACAGATAGGTGTGATGAAGTCACTAAAGTCTTCCT CAGGAATCATATTCAACGCCATTGAAGATCTTGAAACAGATCAGCT TGATGAAGCCCGCATAGAATTCCCAGTTCCACTCTTCTGTATTGGA CCCTTTCACAGGTACGTTTCAGCTTCATCCAGTAGCTTACTTGCAC ACGACATGACTTGTCTCTCCTGGTTAGACAAGCAAGCAACAAATTC CGTAATCTACGCAAGTCTTGGAAGCATTGCTTCGATCGATGAATCT GAATTCTTGGAGATTGCTTGGGGTCTAAGAAACAGCAACCAACCTT TTCTATGGGTGGTTAGACCCGGTTTAATCCACGGGAAAGAATGGAT CGAGATTCTGCCTAAAGGGTTCATCGAAAATCTCGAGGGCCGGGGT AAAATAGTGAAATGGGCACCTCAGCCTGAAGTTTTAGCTCACCGTG CAACAGGCGGATTCTTAACACATTGTGGATGGAACTCAACACTTGA GGGCATATGTGAAGCTATACCAATGATATGCAGACCATCTTTTGGG GACCAGAGGGTGAATGCTAGATACATTAACGATGTTTGGAAGATCG GATTGCATTTGGAAAACAAGGTAGAGAGACTAGTGATCGAAAACGC GGTTAGAACACTAATGACGAGCTCGGAAGGGGAAGAGATCCGCAAG AGGATTATGCCCATGAAGGAAACTGTTGAACAATGCCTTAAGCTTG GAGGTTCATCATTTCGGAATCTCGAAAACTTAATTGCTTATATATT GTCTTTCTAA | 27 |
| Arabidopsis thaliana | AtUGT76B1 | METRETKPVIFLFPFPLQGHLNPMFQLANIFFNRGFSITVIHTEFN SPNSSNFPHFTFVSIPDSLSEPESYPDVIEILHDLNSKCVAPFGDC LKKLISEEPTAACVIVDALWYFTHDLTEKFNFPRIVLRTVNLSAFV AFSKFHVLREKGYLSLQETKADSPVPELPYLRMKDLPWFQTEDPRS GDKLQIGVMKSLKSSSGIIFNAIEDLETDQLDEARIEFPVPLFCIG PFHRYVSASSSSLLAHDMTCLSWLDKQATNSVIYASLGSIASIDES EFLEIAWGLRNSNQPFLWVVRPGLIHGKEWIEILPKGFIENLEGRG KIVKWAPQPEVLAHRATGGFLTHCGWNSTLEGICEAIPMICRPSFG DQRVNARYINDVWKIGLHLENKVERLVIENAVRTLMTSSEGEEIRK RIMPMKETVEQCLKLGGSSFRNLENLIAYILSF | 28 |
| Arabidopsis thaliana | AtUGT76D1 | ATGGCAGAGATTCGCCAGAGAAGAGTGTTGATGGTCCCAGCACCGT TCCAAGGCCATTTACCTTCGATGATGAATCTAGCGTCCTACCTTTC | 29 |
| | | TTCCCAAGGCTTTTCAATCACAATCGTTAGAAACGAATTCAATTTC AAAGATATCTCCCATAATTTCCCTGGTATAAAATTCTTCACCATCA AGGACGGCTTGTCAGAATCTGACGTGAAGTCTCTGGGTCTCCTTGA ATTTGTCCTGGAGCTTAACTCTGTCTGTGAACCCCTATTGAAAGAG TTTCTAACCAACCATGATGATGTTGTTGACTTTATCATTTATGATG AATTTGTTTACTTCCCTCGACGTGTTGCGGAAGATATGAATCTGCC AAAGATGGTCTTTAGCCCTTCTTCCGCCGCTACCTCGATCAGCCGG TGTGTGCTTATGGAGAACCAATCAAATGGGTTACTTCCTCCACAAG ACGCAAGATCTCAACTAGAAGAAACGGTGCCAGAGTTTCATCCCTT TCGTTTCAAAGATCTGCCTTTTACAGCTTATGGATCTATGGAGAGA TTAATGATACTTTACGAGAATGTAAGCAATAGAGCCTCATCTTCTG GCATAATACACAACTCTTCGGATTGCTTAGAGAACTCATTCATAAC AACTGCACAAGAGAAATGGGGAGTTCCGGTATACCCGGTTGGTCCA CTCCATATGACCAATTCCGCAATGTCATGTCCAAGTTTATTTGAAG AAGAAAGAAACTGTCTTGAATGGCTTGAGAAGCAAGAAACAAGCTC AGTGATCTACATAAGCATGGGGAGCTTGGCGATGACACAAGATATA GAGGCTGTGGAGATGGCCATGGGATTTGTCCAGAGTAATCAACCCT TCTTGTGGGTGATCCGACCAGGCTCTATAAACGGACAAGAATCTTT AGACTTCTTACCGGAACAGTTCAACCAAACGGTGACCGATGGAAGA GGTTTTGTTGTGAAATGGGCCCCACAAAAAGAGGTATTAAGGCATA GAGCAGTGGGAGGGTTTTGGAACCATGGTGGATGGAACTCGTGCTT GGAGAGCATAAGCAGTGGTGTACCAATGATTTGTAGGCCGTATTCT GGTGATCAGAGGGTGAATACTCGACTTATGTCACATGTTTGGCAAA CCGCGTATGAGATCGAAGGTGAATTGGAAAGAGGAGCTGTTGAGAT GGCCGTGAGGAGGCTCATTGTGGATCAAGAAGGTCAGGAGATGAGA ATGAGAGCCACCATATTGAAGGAAGAGGTTGAAGCCTCTGTCACAA CCGAAGGCTCTTCTCACAATTCTTTAAACAATTTGGTCCATGCAAT AATGATGCAAATTGACGAACAATGA | |
| Arabidopsis thaliana | AtUGT76D1 | MAEIRQRRVLMVPAPFQGHLPSMMNLASYLSSQGFSITIVRNEFNF KDISHNFPGIKFFTIKDGLSESDVKSLGLLEFVLELNSVCEPLLKE FLTNHDDVVDFIIYDEFVYFPRRVAEDMNLPKMVFSPSSAATSISR CVLMENQSNGLLPPQDARSQLEETVPEFHPFRFKDLPFTAYGSMER LMILYENVSNRASSSGIIHNSSDCLENSFITTAQEKWGVPVYPVGP LHMTNSAMSCPSLFEEERNCLEWLEKQETSSVIYISMGSLAMTQDI EAVEMAMGFVQSNQPFLWVIRPGSINGQESLDFLPEQFNQTVTDGR GFVVKWAPQKEVLRHRAVGGFWNHGGWNSCLESISSGVPMICRPYS GDQRVNTRLMSHVWQTAYEIEGELERGAVEMAVRRLIVDQEGQEMR MRATILKEEVEASVTTEGSSHNSLNNLVHAIMMQIDEQ | 30 |
| Cannabis sativa | CsUGT75B2 | ATGGTTCAGCCAAGATTCTTGATTTTGGCTTTTCCATTGCAGGGTA CTATTAACCCATGTTTGAACTTGGCTAACCAGTTGATTAGAGTTGC TAACGCTCAAGTTACTTTCGTTACTTCTGTTAACGCCCACAGATTG ATTATGACTACTCATACTGTTGCTACCACCTCCAACAATTTGTTGT CTTTTTCTCCATTCTTCGACGGTTACGATGAAGGTGTTACTGATGG TAAAGGTTTCCATGATCACTTCGTCGAATTCAAAAGAAGAGGTTGG CAAGCTGTTGGTGATATTTTGGAATTGGGTTTCAAAGAAGGTAGGC CATACACTTGTTTGGTCTACTCTATTTTGTTGACTTGGGCTGCTGA TGTTGCTGCTACACATAATGTTCCAGCTTCTATGTTTTGGATGCAA CCAGCTACTGTTTTCGATGTGTATTACTACTACTTCCACGGCCACA AAGAAATTATCTGTGCTAACACTAAGAACCACAGCTTCTCATTGTC TTTCCCAAGAATTCCATTGACCATGAACTTGAAGGATCTGCCATCT TTGATGGTTGACTCTAACTACTCTTACATCTTGACCATGTTGCACG AAATGTACAAGGACTTCGAAAAAGAGTCTAACAACACCAAGATCAT CCTGGTTAACACTTTCGATGAATTGGAACCAGATGCTTTGAGAGCC ATTAACAAGTTCAACTTGATTGGTATCGGTCCCTTGATTACTTCTA AGACCTCATTCTCTTTCAGAAACTACATCGAATGGTTGAACACGAA GCCAAAAAAGACCGTTGTTTACGTTTCCTTCGGTTCCATTCTGATC TTGAAAAAACAACAGATGGACGAAATTGCCAAGGGTTTGTTGGAAT TTGGTCATCCATTTCTGTGGGTCATCAAAGAGAAGAACTCCTCATC TAAAGAAGGTGTCCACGAAGATAACATCAAGAACGAGTTGTCCTAC AAAGAGGAATTGGAAAAGTTGGGTATGATCGTTCCATGGTGTTCTC AAATGGAAGTGTTGAGAAATGAATCCTTGGGTTGTTTCGTTACACA TTGTGGTTGGAACTCTACCTTGGAATCTATAGTTTCTGGTGTTCCA GTTGTTGCTTTTCCACAATGGACTGATCAACAAACAAACGCCAAGT | 31 |
| | | TGATTGAAGAGATGTGGAAGATTGGTGTCAGAGTTAAGCCAGATGA AGATGGTATTGTCAAGTCCGAAGAAATCAAGAGATGTTTGGAGTTG GTCATGTCCAAGAACGAAAACAGAACTGAAATCGTGAAGAACGTCA AGAAGTGGCAAAACTTGACAAAAGAAGCTATGAGAGAAGGCGGTTC TTCTGAAAAAAACTTGATCACCTTCGTGAAGTCCATCCACCAATGA | |
| Cannabis sativa | CsUGT75B2 | MVQPRFLILAFPLQGTINPCLNLANQLIRVANAQVTFVTSVNAHRL IMTTHTVATTSNNLLSFSPFFDGYDEGVTDGKGFHDHFVEFKRRGW QAVGDILELGFKEGRPYTCLVYSILLTWAADVAATHNVPASMFWMQ PATVFDVYYYYFHGHKEIICANTKNHSFSLSFPRIPLTMNLKDLPS LMVDSNYSYILTMLHEMYKDFEKESNNTKIILVNTFDELEPDALRA INKFNLIGIGPLITSKTSFSFRNYIEWLNTKPKKTVVYVSFGSILI LKKQQMDEIAKGLLEFGHPFLWVIKEKNSSSKEGVHEDNIKNELSY KEELEKLGMIVPWCSQMEVLRNESLGCFVTHCGWNSTLESIVSGVP VVAFPQWTDQQTNAKLIEEMWKIGVRVKPDEDGIVKSEEIKRCLEL VMSKNENRTEIVKNVKKWQNLTKEAMREGGSSEKNLITFVKSIHQ | 32 |
| Cannabis sativa | CsUGT75B4 | ATGTCCAAGGGTCATACCATTCCATTATTGCATTTGGCCAGAGTCT TGTTGAACAGACATGTTACTGTTACCATTTTCACTACCCCAGCTAA CAGATCTTTCATCACTAAGTTTTTGCCAGCTACTTCTGCTGCTATA GTCGAATTGCCATTTCCAAAGAATATTCCAGGTGTTCCAAACGGTG TTGAAAACACTGAAGATTTCCCAACCATGTCCATGTCTATGTTCTA CTCATTGGTTTTGGGTACGCAGAATATGAAGCCAGATTTGGATAGA GCCTTGGAAAACATTCAAACCCCAGTTTCTTTCATGGTTTCCGATG GTTTTTTGTGGTGGACTTTGGATTCTGCTTCTAAGTTGGGTTTTCC CAGATTGGTTTTTTACGGTATGTCTCATTATGCCATGGCCGTTTAC CATTCTTTGTTCAATTCTAAGAAGCCAAAGCAGACTGAAACCGAAA CTGTTGTTTCTGATTTCCCATGGATTAAGTTGACCAGATCTGAATA TGATCCATCCGCTCAAAATGGTGAAGATCAATCTTTGGCTCACGAG TTTATGTCTAAAGCTACTGAAGCTACCAACAACTCTTTCGGTATGA TCTACAACTCCTTCTACGAATTGGAACCTATGTTCACCGATTACTG GAATCAAAAAGTTGGTCCAAAATCTTGGCCATTGGGTCCATTGTGT TTACACGATATGAAGATCGAATCCAGAGGTTTGGTTGTTCATCCAT GGTTGGACGAAAAAGAATCTTCTTCTGTCTTGTACGTTGCCTTTGG TTCTCAAGCTACTGTTTCTTCTGAACAGGTTAGAGAAATTGCCAAA GGTTTGGAATACTCCAACGTTAACTTTTTCTGGGTCTTGAGAAAGT TGGAACCAGAAGAAAACAAGTTCTTGGAAGAATTCGAAAAGAGGGT CAAGAACAGAGGTATCGTTGTTAGAGATTGGGTTAACCAGATGGAA ATCTTGAAACACAAGTCCGTTAAGGGTTTCTTCTCTCATTGTGGTT GGAACTCTGTTATGGAATCTTTGTCTGCTGGTTTGCCAATTTTGGG TTTCCCAATGATGGCTGAACAACATATTAACGCCAAGATGGTTGTC GAAGAAATCAAGATAGGTCTGAGAGTTAAGTCCTACGATGGTTCTT TGAATGGTATCGTTAAGTCCGAAGAGGTTAGCAAGATGGTCAAAGA ATTGATGGAAGGTGAAGTCGGTAAAGAGATGAGAAAGAAGGTTGAA GAATTTGCTGTTATGGCTCATAAGGCCGTTCAAAAAGGTGGTTCTT CTTGGGAAACTTTGGACTTGTTGTTGTCCTCTACCAAGCAAAGAAT CCATCACTACTGA | 33 |
| Cannabis sativa | CsUGT75B4 | MSKGHTIPLLHLARVLLNRHVTVTIFTTPANRSFITKFLPATSAAI VELPFPKNIPGVPNGVENTEDFPTMSMSMFYSLVLGTQNMKPDLDR ALENIQTPVSFMVSDGFLWWTLDSASKLGFPRLVFYGMSHYAMAVY HSLFNSKKPKQTETETVVSDFPWIKLTRSEYDPSAQNGEDQSLAHE FMSKATEATNNSFGMIYNSFYELEPMFTDYWNQKVGPKSWPLGPLC LHDMKIESRGLVVHPWLDEKESSSVLYVAFGSQATVSSEQVREIAK GLEYSNVNFFWVLRKLEPEENKFLEEFEKRVKNRGIVVRDWVNQME ILKHKSVKGFFSHCGWNSVMESLSAGLPILGFPMMAEQHINAKMVV EEIKIGLRVKSYDGSLNGIVKSEEVSKMVKELMEGEVGKEMRKKVE EFAVMAHKAVQKGGSSWETLDLLLSSTKQRIHHY | 34 |
| Cannabis sativa | CsUGT73B1 | ATGTCCAAAGAAATCTGGGTTGTTCCATTCTTTGGTCAAGGTCATT TGTTCCCATCTATGGAATTGTGCAAGCAAATTGCCTCCAGAAACAT TAACACCTTGTTGGTTATTCCCTCCAACCTGTCTTTTTCTATCCCA TCTTCTTTGAGACAATACCCCTTGTTGCAAATCGTTGAAATTCAAC CTACTTCTGCTCCATCTGCTCAACCAGGTCCAGATCCAATTGATCA ACCACCACATGGTAATCCAGATCAATTCGAAATGGGTCTAGAAAAC TTGTTGCAGGCTCAAGTTACTGGTCCAGATTCTGTTAGACCAATTT GCGCTATTTTGGATGTTATGATGGATTGGACTACCGAGGTTTTCAA | 35 |
| | | CAAGTTCGATATTCCAACTATCGGCTTCTTTACTTCTGGTGCTTGT TCTGCTGCTATGGAATATGGTTTGTGGAAAGCTCAACCTATCGATT TGAAACCAGGTGAAGTTAGATTATTGCCAGGTTTGCCAGAAGAAAT GGCTGTTACTTACTTGGACACTAATCAAAGACCACATCAACCTCCA GGTCCACCATTGCATTTGTTTGGTGTTGGTCATCAAGGTCCTTTTG TTGATGGTGCTCATAGACCACCAGGTCCTCCACCACCACCATATTT GGGTAGAGCTGGTCCAAGACAAAGGGGTCCACCAAAACATGGTTCA CAACCACCATGGGTTGATGGTTTGAAAGGTTCTATTGCCTTGATGA TTAACACGTGCGATTACTTGGAAAGGCCATTCATTGAATACTTGAC CAAGCAAATCGGTAAACCAGTTTGGGGTGTAGGTCCATTATTACCA GAACAATTTTGGAACTCCGTGTCCTCTAATTCCATATTGCATGATC ACGAAATCAGGACCAACAGACAATCTAACGTTTCCGAAGATGATGT CATCCAATGGTTGGATTCTAAACCTAGAGGTTCTGTCTTGTACGTT TGTTTCGGTACTGAAGTTTCTCCATCCATGGAAGAATACTCTGAAT TGGCTGATGCTTTGGAAGCTTCTACTCAACCTTTTATTTGGGTCGT TCATTCTGGTACTGGTAGAAGTGGTCCACCACCTTCTAGAGGTCCA ACTCAAGAGGATTATTTTCCACATGGTTTGGCTTCTCAAGTTGGTC CAAAAGGTTTGATTATTAACGGTTGGGCTCCACAGTTGTTGATCTT GTCTCATTCTTCTATTGGTGGTTTCTTGACTCATTGTGGTTGGAAC TCTACTGTTGAAGCTATTGGTTTAGGTGTTCCATTATTGGCTTGGC CAATTAGAGGTGATCAAAACTACAATGCCAAGTTGGTTGTTGCCCA TTTGAAGTTGGGTTTCATGATCTCTGATAACCTGTCCGAGAAGATT AAGAAGCACGAAATTGTCAAGGGTATCAAGACTTTGATGGGTGATG ATGATATTAAGTCCAGAGCTAGAAACTTGGCTGCCATTTTTCAAAA GGGTTTCCCAATTTCTTCTACCACTAACTTGGATGTGTTCAGGGAT ATGATCAACAATTTCACGTAA | |
| Cannabis sativa | CsUGT73B1 | MSKEIWVVPFFGQGHLFPSMELCKQIASRNINTLLVIPSNLSFSIP SSLRQYPLLQIVEIQPTSAPSAQPGPDPIDQPPHGNPDQFEMGLEN LLQAQVTGPDSVRPICAILDVMMDWTTEVFNKFDIPTIGFFTSGAC SAAMEYGLWKAQPIDLKPGEVRLLPGLPEEMAVTYLDTNQRPHQPP GPPLHLFGVGHQGPFVDGAHRPPGPPPPPYLGRAGPRQRGPPKHGS QPPWVDGLKGSIALMINTCDYLERPFIEYLTKQIGKPVWGVGPLLP EQFWNSVSSNSILHDHEIRTNRQSNVSEDDVIQWLDSKPRGSVLYV CFGTEVSPSMEEYSELADALEASTQPFIWVVHSGTGRSGPPPSRGP TQEDYFPHGLASQVGPKGLIINGWAPQLLILSHSSIGGFLTHCGWN STVEAIGLGVPLLAWPIRGDQNYNAKLVVAHLKLGFMISDNLSEKI KKHEIVKGIKTLMGDDDIKSRARNLAAIFQKGFPISSTTNLDVFRD MINNFT | 36 |
| Cannabis sativa | CsUGT75D 1_DN11028 | ATGAAGAGGACCTTGTTGTTTATTCCATCTCCAGGTATTGGTCACC TGGTTTCTATGTTGGAATTTGCCAAGAGATTGATCCAATACGATGA CAGGTTGTTCATCACCATCTTGTCTATGAAGTTCCCAAACCATGAT GCCTACATCAATTCTTTGGTTCCATCCTTGTCTCAGTCCAGAGTTA AGTTGGCTCATTTGCCACAAGTTGATCCTCCACCACCAAAGTTGTT GAATTCTCCAGAATCTTACATCTACGTCTACGTCGAATCTTTAGTT CCACATGTTAGAGATGCTTTGAAGCACATAGTTCCATCTCACTCTA ACTCTGAAACTACCCATTCTCAAGGTTGCTTCGTTATGGTTTTGGA TTTCTTCTGTATGCCAATGATGGATGTTGCTAACGAATTGGGTTTG CCATCTTACATGTTCATGCCATCTAACATCGGTTTCTTGTCCTCTA TGTTGTACTTGGCTACTAGACACGATCAGATCAGCTCTGAATTGAA AGAATCTAACCCAGACGAGTACTCCTTGAAGTCTTTTCATAATCCA GTTCCATGGTCTGCTTTACCTCAAGCTTATTTCTGTAAAGACGGTG GTTATTCTGCTTGCGTAAAAATGGCTCAAAGATTCAGAGAAACTAA GGGCATCATCGTCAATTCCTTTGAAGGTTTGGAAGCTCATGGTGCT ACATCTTTTAATGATGGTGAAACTCCACCAATCTACATGGTTGGTC CAGTTGTTAATTTCAAGGGTCAACCACATTCTTCCACTGATCATGT TCAAAACAACAGGATCTTCAAGTGGTTGGACGAACAACCACAATCC TCAGTTGTTTTTTTGTGCTTTGCTTCCTTGGGTACTTTCGATGCTT CACAATTGAGAGAAATTGCCTCTGGTTTGGAATGTTCTGGTCATAG ATTTTTGTGGTGCATCAGAGTTCAACAGCCAACCATTATTGACGAA ATTTTGCCAGAAGGTTTCTTGGAAAGAATCGGTTCTAAAGGTATGA TCTGTAACGAATGGACTCCACAAGTAGAAGTTTTGGCTCATAATGC TGTTGGTGGTTTCGTTTCTCATTGCGGTTGGAATTCAATCTTGGAA TCTTTGTGGTATGGTGTTCCAATAGTTACTTGGCCAGTTTACGCTG | 37 |
| | | AACAACAATTGAATGCTTTCCAGATGGTTAGGGAATTCGATTTGGC TATCGAATTGAGATTGGACTACAGAAACAGAGGTCATAACCAGTTG GTTACCGCTGAAGAAATTGGTAACGCCATCAAAAAATTGATGGAAG GTGATCACAACGTCGTCATGAGAAAAAAAGGTGCTTCTGGTATCTC TAGCATGGTCTTGTCTAGATTCTACACCTGTAAAGAAAACGTGGTC TTGAGACAAGATCCAAGATGGTTTGCTATTAAGGCTGGTGGTGAAG AAAAGTAA | |
| Cannabis sativa | CsUGT75D 1_DN11028 | MKRTLLFIPSPGIGHLVSMLEFAKRLIQYDDRLFITILSMKFPNHD AYINSLVPSLSQSRVKLAHLPQVDPPPPKLLNSPESYIYVYVESLV PHVRDALKHIVPSHSNSETTHSQGCFVMVLDFFCMPMMDVANELGL PSYMFMPSNIGFLSSMLYLATRHDQISSELKESNPDEYSLKSFHNP VPWSALPQAYFCKDGGYSACVKMAQRFRETKGIIVNSFEGLEAHGA TSFNDGETPPIYMVGPVVNFKGQPHSSTDHVQNNRIFKWLDEQPQS SVVFLCFASLGTFDASQLREIASGLECSGHRFLWCIRVQQPTIIDE ILPEGFLERIGSKGMICNEWTPQVEVLAHNAVGGFVSHCGWNSILE SLWYGVPIVTWPVYAEQQLNAFQMVREFDLAIELRLDYRNRGHNQL VTAEEIGNAIKKLMEGDHNVVMRKKGASGISSMVLSRFYTCKENVV LRQDPRWFAIKAGGEEK | 38 |
| Cannabis sativa | CsUGT71D 1_DN48028 | ATGGCCAGAGTCGAATTGGTTTTTATTCCAGCTCCAGCTATTGGTC ATTTGGTTTCTACTTTGGAATTCGCCAAGAGATTGATCCATTACGA TCATAGGTTGTTCATCACCGTTTTGTGCGAATTCTCTTTGAAGTCT CATTTGGATGCCTACATCGATTCTTTGGTTGCTTCTTTGTCTTTGG CCCATAGAATCAAGTTGGTTCATTTGCCATTGGTTGATTCTCCACC AGTTGAGTTGTTGAAGTCCATTGAAAATTTCATCTACCAGTACATG GAAAGCTTGGTCCCACATGTTAGAAAAGCTTTGACTGACATCGTGT CCTCTAACTCTAATTACTCTCAAGGTGATGTTGTCTTGGTCTTGGA TTTTTTCTGTATGCCAATGATGGATGTCGCTAACGAATTGGGTTTG CCATCTTACATGTTCATGACTTCTAACTTGGGCCTGTTGTCTTTGA TGTTTTACTTGGCTACCAGACACAACCAGATCTCTTCAGAATTGGA AGAATCTGATGCTCCATTGAGATTGCAAGGTTTTCAAAATCCAGTT CCATCCTCTGTTTTGCCAACTGCTGCTTTCTGTAAAGATGGTGGTT ATTCTGCTTACGTTAAGTTGGCTCAAAGATTCAGAGAAACTAAGGG CATCATCGTCAACTCATTTGAAGAATTGGAGTCCTACTCCTTCTCC TCTATGAATGATGGTGCTGAAACTCCACCAATCTATATGGTTGGTC CAGTTTTGGATTTGAACGGTCAACCACATCCATCTATGGATCAAGT TCAAAACGACAAGATCCTGAAGTGGTTGGACGAACAACCTCATTCT TCTGTTGTTTTCTTGTGCTTTGGCTCCATGGGTAAATTTGGTGCTT CACAATTGAGAGAAATTGCCTCCGGTTTACAAAGATCTGGTCATAG ATTTTTGTGGTCCGTTAGAGTTCAACAACCTACTACCATTGACGAA ATTTTGCCAGAAGGTTTCTTGGAACAAATCGGTTCTAAAGGTATGA TCTGTAACGAATGGGCTCCACAAGTTAAGATTTTGGCTCATTCAGC TGTTGGTGGTTTCTTGTCTCATTGTGGTTGGAACTCTATCTTGGAA TCTTTGTGGTATGGTGTTCCAGTTGCTACTTGGCCAATCTATGCTG AACAACAATTGAACGCTTTCAGGATGGTTAGAGAATTTGGTTTGGC TGTTGATTTGAGGTTGGATTACAAGGATAGAGGTGATGATCATATC GTTTCCGCCGAAGAAATTGAAACTGCTGTCAAACATTTGATGGAAG GTGACAAAGAGGTCCGTAAAAAGGTCAAAGAAATGTCTGAAACCGC CAGAAAGTCTGTTGAAGAAGGTGGTTCTTCTTTCACCGCTATTGGT AAATTGATCAACTCCATCATCGGCTCCAATTACTACTGA | 39 |
| Cannabis sativa | CsUGT71D 1_DN48028 | MARVELVFIPAPAIGHLVSTLEFAKRLIHYDHRLFITVLCEFSLKS HLDAYIDSLVASLSLAHRIKLVHLPLVDSPPVELLKSIENFIYQYM ESLVPHVRKALTDIVSSNSNYSQGDVVLVLDFFCMPMMDVANELGL PSYMFMTSNLGLLSLMFYLATRHNQISSELEESDAPLRLQGFQNPV PSSVLPTAAFCKDGGYSAYVKLAQRFRETKGIIVNSFEELESYSFS SMNDGAETPPIYMVGPVLDLNGQPHPSMDQVQNDKILKWLDEQPHS SVVFLCFGSMGKFGASQLREIASGLQRSGHRFLWSVRVQQPTTIDE ILPEGFLEQIGSKGMICNEWAPQVKILAHSAVGGFLSHCGWNSILE SLWYGVPVATWPIYAEQQLNAFRMVREFGLAVDLRLDYKDRGDDHI VSAEEIETAVKHLMEGDKEVRKKVKEMSETARKSVEEGGSSFTAIG KLINSIIGSNYY | 40 |

In at least one embodiment, the foregoing methods for synthesizing a glycosylated cannabinoid or cannabinoid precursor using a UGT catalyzed reaction can be carried out in vitro. Thus, in at least one embodiment, the reaction constituents, i.e., a cannabinoid compound, a glycosyl group containing substrate, and a glycosyl transferase are contacted in an aqueous solution contained in a suitable reaction vessel, e.g., a tube, a bottle, or a dish. Reaction conditions suitable for carrying out such in vitro enzymatic reactions are well known in the art, and generally approximate physiological conditions. Furthermore, those of skill in the art will be able to modulate or optimize reaction conditions, for example, by preparing multiple reaction vessels, performing the in vitro reaction under multiple reaction conditions and evaluating the formation of glycosylated cannabinoid compound under these different reaction conditions. Subsequently a desired reaction condition may be selected.

In at least one embodiment, in vitro reaction conditions useful in the methods of the present disclosure can include, for example, 50-200 mM NaCl or KCl, pH 6.5-8.5. 20-45° C., or 30-40° C., and 0.001-10 mM divalent cation (e.g., Mg⁺⁺, Ca⁺⁺). In some embodiments, suitable in vitro reaction conditions can comprise about 150 mM NaCl or KCI, pH 7.2-7.6, 5 mM divalent cation, and often include 0.01-1.0 percent nonspecific protein (e.g., BSA). Additionally, a non-ionic detergent (Tween. NP-40, Triton X-100) can often be present, usually at about 0.001 to 2%, or typically 0.05-0.2% (v/v). Particular aqueous conditions may be selected by the practitioner according to conventional methods. For example, some other buffered aqueous conditions suitable for use in the methods of the present disclosure may include 10-250 mM NaCl, 5-50 mM Tris HC1, pH 5-8, with optional addition of divalent cation(s) and/or metal chelators and/or non-ionic detergents and/or membrane fractions and/or anti-foam agents and/or scintillants. Generally, in carrying out an in vitro reaction, all reaction constituents are mixed, for example by gentle stirring or shaking the reaction vessel. Reaction times may vary, but generally the glycosylated cannabinoid compound can be formed in less than about 30 minutes, for examples less than about 20 minutes, or less than about 5 minutes.

In at least one embodiment, the foregoing methods for synthesizing a glycosylated cannabinoid or cannabinoid precursor using a UGT catalyzed reaction can be carried out in vivo, that is in a recombinant host cell. In such in vivo embodiments, the enzymatic reaction involving contacting a UGT with a glycosyl group bearing substrate and a cannabinoid or a cannabinoid precursor acceptor under suitable reaction conditions comprises in vivo conditions that comprise growing a recombinant host cell comprising a heterologous nucleic acid that encodes the UGT. The growth of the recombinant host cell thereby results in expression of the UGT. In one such in vivo embodiment, it is contemplated that the recombinant host cell expresses the UGT into a culture medium comprising a glycosyl group bearing substrate and a cannabinoid or a cannabinoid precursor acceptor, whereby the glycosylated cannabinoid or cannabinoid precursor compound is produced in the medium.

As described elsewhere herein, a number of UGTs produced by the plant source organisms Arabidopsis thaliana and Helianthus annuus have been identified as capable of catalyzing the glycosylation of cannabinoids. Accordingly, the in vivo embodiments contemplate that the heterologous nucleic acid encoding a UGT in the recombinant host cell can comprise an amino acid sequence having at least 90% identity to a sequence selected from SEQ ID NO: 2, 4, 6, 8, 10, 12, 14, 16, or 18; or that the heterologous nucleic acid itself comprises a nucleotide sequence having at least 90% identity to a sequence selected from SEQ ID NO: 1, 3, 5, 7, 9, 11, 13, 15, or 17.

The present disclosure also provides an in vivo method wherein the recombinant host cell that further comprises a pathway capable of producing the cannabinoid or the cannabinoid precursor compound that undergoes UGT-catalyzed glycosylation. For example, the recombinant host cell can be a prokaryote, such as E. coli, or a eukaryote, such as S. cerevisiae, that has previously been engineered with heterologous nucleic acids encoding a pathway of enzymes capable of converting a carbon source, such as glucose, into a cannabinoid precursor, such as olivetolic acid, and then into a cannabinoid, such as CBGA. Accordingly, in at least one embodiment, the in vivo method comprises growing a recombinant host cell engineered to express a UGT, and also engineered with a pathway comprising enzymes capable of converting hexanoic acid to the cannabinoid precursor compound, olivetolic acid. For example, a recombinant host engineered to express a pathway of enzymes capable of catalyzing the reactions (i) - (iii) from hexanoic to olivetolic acid shown below:

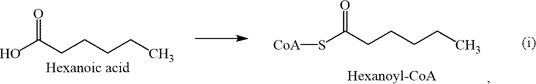

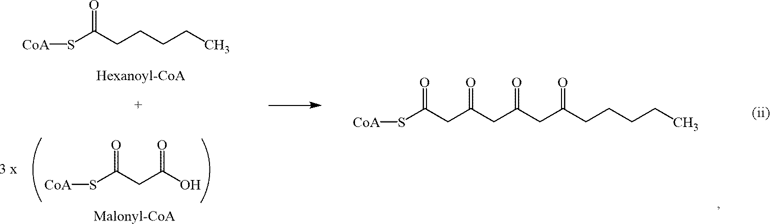

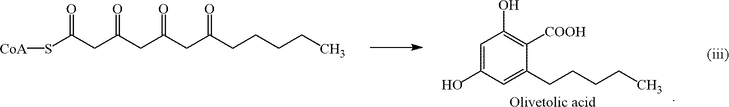

The present disclosure also contemplates that the recombinant host cell engineered with a pathway from hexanoic acid to olivetolic acid can also be engineered to express an enzyme capable of converting olivetolic acid and geranyldiphosphate to the cannabinoid compound, cannabigerolic acid, CBGA. For example, the recombinant host cell can further express enzyme capable of catalyzing reaction (iv) below:

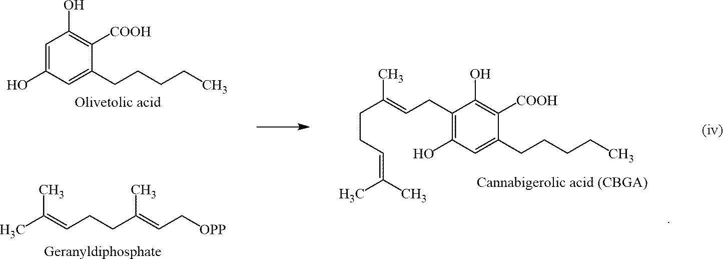

As described elsewhere herein, enzymes capable of catalyzing the reactions (i) -(iv) have been identified and isolated from C. sativa and other organisms, and engineered for recombinant expression in microorganisms, such as yeast. For example, in one embodiment of the method comprising a recombinant host cell engineered with a pathway capable of producing the cannabinoid or the cannabinoid precursor, the pathway can comprise at least the enzymes, AAE, OLS, and OAC, having amino acid sequences of at least 90% identity to SEQ ID NO: 82 (AAE), SEQ ID NO: 84 (OLS), and SEQ ID NO: 86 (OAC), respectively. In at least one embodiment, the engineered pathway can further comprise a prenyltransferase, PT4 having at least 90% identity to SEQ ID NO: 88 or 90.

In at least one embodiment, the in vivo methods of the present disclosure can comprise a recombinant host cell with a pathway that further comprises an enzyme capable of catalyzing the conversion of the cannabinoid, CBGA to Δ⁹-THCA, or CBDA, or CBCA. For example, a pathway comprising enzymes capable of catalyzing the conversions (i) - (iv) as described above, can further comprise an enzyme capable of catalyzing a reaction (v), (vi), and/or (vii):

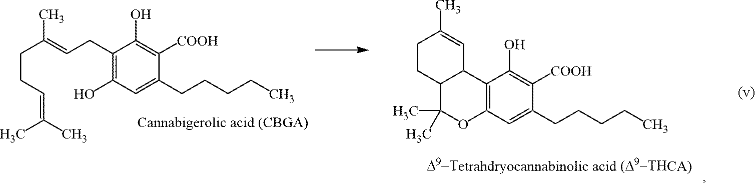

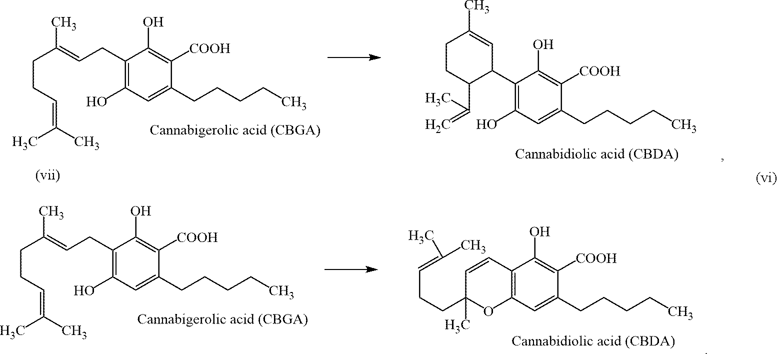

Enzymes capable of catalyzing the conversions (v), (vi), and (vii), have been identified and isolated from C. sativa, and include THCA synthase, CBDA synthase, and CBCA synthase. For example, in at least one embodiment, the recombinant host cell can comprise a pathway that expresses CBDA synthase having an amino acid sequence of at least 90% identity to SEQ ID NO: 12 or 14.

In at least one embodiment, the present disclosure provides an in vivo method of producing a glycosylated cannabinoid or glycosylated cannabinoid precursor compound that comprises: (a) providing a nucleic acid sequence comprising as operably linked components (i) a first nucleic acid sequence encoding a UGT; and (ii) a second nucleic acid sequence capable of controlling expression in a host cell; (b) introducing the nucleic acid sequence into a host cell having a pathway capable of producing a cannabinoid precursor, and optionally capable of producing a cannabinoid; and (c) growing the host cell under conditions in which the host cell expresses the UGT and produces a cannabinoid precursor and/or cannabinoid compound, and in which the UGT produced by the host cell glycosylates the cannabinoid and/or cannabinoid precursor compound.

Preparation of a recombinant host cell capable of being used in such an embodiment initially involves providing a nucleic acid sequence encoding a UGT and introducing the heterologous nucleic acid sequence encoding the UGT into host cells. Accordingly, next example chimeric nucleic acids and example host cells that may be selected and used in accordance with the present disclosure will be described. Thereafter example methodologies and techniques will be described to produce example glycosylated cannabinoid compounds in vivo.

Nucleic acid sequences that may be used include any nucleic acid encoding a glycosyl transferase capable of glycosylating a cannabinoid compound, including, without limitation, the exemplary nucleic acid sequences set forth herein. In at least one embodiment, a nucleic acid encoding a glycosyl transferase that may be used in accordance with the present disclosure include
(a) a nucleic acid sequence that is substantially identical to any one of the nucleic acid sequences having SEQ ID NO: 1, 3, 5, 7, 9, 11, 13, 15, or 17;
(b) a nucleic acid sequence that is substantially identical to any one of the nucleic acid sequences having SEQ ID NO: 1, 3, 5, 7, 9, 11, 13, 15, or 17; but for the degeneration of the genetic code;
(c) a nucleic acid sequence that is complementary to any one of the nucleic acid sequences having SEQ ID NO: 1, 3, 5, 7, 9, 11, 13, 15, or 17;
(d) a nucleic acid sequence encoding a polypeptide having any one of the amino acid sequences set forth in SEQ ID NO: 2, 4, 6, 8, 10, 12, 14, 16, or 18;
(e) a nucleic acid sequence that encodes a functional variant of any one of the amino acid sequences set forth in SEQ ID NO: 2, 4, 6, 8, 10, 12, 14, 16, or 18; and
(f) a nucleic acid sequence that hybridizes under stringent conditions to any one of the nucleic acid sequence having SEQ ID NO: 1, 3, 5, 7, 9, 11, 13, 15, 17, or those set forth in (a), (b), (c), (d), or (e).

The second nucleic acid sequence capable of controlling expression in the host cell includes any transcriptional promoter capable of controlling expression of polypeptides in host cells. Generally, a transcriptional promoter is selected to be compatible with the host cell, so that promoters obtained from bacterial cells are used when a bacterial host cell is selected in accordance herewith, while a fungal promoter is used when a fungal host cell is selected, a plant promoter is used when a plant cell is selected, and so on. Promoters may be constitutive or inducible, provided such promoters are operable in the host cells. Example promoters that may be used to control expression in bacterial cells include Escherichia coli promoters such as a lac, tac, trc, trp or T7 promoter. Promoters that may be used to control expression in fungal cells include a Saccharomyces cerevisiae inducible promoter, such as a GAL1 promoter or GAL10 promoter, a constitutive promoter, such as an alcohol dehydrogenase (ADH) promoter or a glyceraldehyde-3-phosphate dehydrogenase (GPD) promoter, or an S. pombe Nmt, or ADH promoter. Examples of promoters that may be used to control expression in plant cells include, for example, a Cauliflower Mosaic Virus 35S promoter (Odell et al. (1985) Nature 313:810-812), a ubiquitin promoter (U.S. Pat. No. 5,510,474; Christensen et al. (1989)), or a rice actin promoter (McElroy et al. (1990) Plant Cell 2:163-171). Examples of promoters that can be used in mammalian cells include, for example, a viral promoter such as an SV40 promoter or a metallothionine promoter. All of these promoters are readily available to the art. Further nucleic acid elements capable elements of controlling expression that in a host cell include transcriptional terminators, enhancers and the like, all of which may be included in the chimeric nucleic acid sequences of the present disclosure.

In accordance with the present disclosure a first nucleic acid sequence encoding a UDP glycosyl transferase is linked to a second nucleic acid sequence capable of controlling expression in a host cell. As will be known to those of skill in the art, a wide variety of techniques for linking nucleic acid sequences to thereby create a chimeric nucleic acid sequences is available. They are for example described in: Sambrook et al., Molecular Cloning, a Laboratory Manual, Cold Spring Harbor Laboratory Press, 2012, Fourth Ed.

A variety of host cells useful in the context of the methods and compositions of the present disclosure, including microbial host cells, plant host cells, and animal host cells. In some embodiments, the host cell can be a microbial cell such as a bacterial cell (e.g., Escherichia coli) or a fungal cell, such as a yeast cell (e.g., a Saccharomyces cerevisiae, or Yarrowia lipolytica). Other cells are contemplated including an algal cell, or a plant cell, suitable cells obtainable from plants belonging to the plant families of Cannabaceae, and further including plants belonging to the genus Cannabis, including Cannabis sativa.

Nucleic acid sequences encoding cannabinoid pathway polypeptides, and related polypeptide sequences are well known to those of skill in the prior art and thus can readily be selected and used in accordance with the present disclosure. Typically, the nucleic acid sequence encoding enzymes which form a part of a cannabinoid pathway, further include one or more additional nucleic acid sequences, for example, a nucleic acid sequence controlling expression of the proteins which form a part of a cannabinoid biosynthetic enzyme complement, and these one or more additional nucleic acid sequences together with the nucleic acid sequence encoding a protein which form a part of an cannabinoid biosynthetic enzyme complement can be said to form a chimeric nucleic acid sequence.

A variety of techniques and methodologies to manipulate host cells to introduce nucleic acid sequences in host cells and attain expression of a UGT, and optionally, depending on the selected cells, to introduce nucleic acid sequences encoding the cannabinoid biosynthetic enzyme complement and attain expression thereof, exist and are well known to the skilled artisan and can, for example, be found in Sambrook et al., Molecular Cloning, a Laboratory Manual, Cold Spring Harbor Laboratory Press, 2012, Fourth Ed.

Nucleic acid sequences capable of controlling expression in host cells that may be used herein include any transcriptional promoter capable of controlling expression of polypeptides in host cells, and are known to the art. Furthermore, some example promoter sequences have hereinbefore been referenced.

In accordance with the present disclosure, chimeric nucleic acid sequences comprising a promoter capable of controlling expression in host cell linked to a nucleic acid sequence encoding a UDP glycosyl transferase, and, as necessary, other polypeptides constituting a cannabinoid biosynthetic enzyme complement, can be integrated into a recombinant expression vector which ensures good expression in the host cell, wherein the expression vector is suitable for expression in a host cell. The term “suitable for expression in a host cell” means that the recombinant expression vector comprises the chimeric nucleic acid sequence linked to genetic elements required to achieve expression in a cell. Genetic elements that may be included in the expression vector in this regard include a transcriptional termination region, one or more nucleic acid sequences encoding marker genes, one or more origins of replication, and the like. In some embodiments, the expression vector further comprises genetic elements required for the integration of the vector or a portion thereof in the host cell’s genome, for example if a plant host cell is used the T-DNA left and right border sequences which facilitate the integration into the plant’s nuclear genome.

Pursuant to the present disclosure, the expression vector may further contain a marker gene. Marker genes that may be used in accordance with the present disclosure include all genes that allow the distinction of transformed cells from non-transformed cells, including all selectable and screenable marker genes. A marker gene may be a resistance marker such as an antibiotic resistance marker against, for example, kanamycin or ampicillin. Screenable markers that may be employed to identify transformants through visual inspection include β-glucuronidase (GUS) (U.S. Pat. Nos. 5,268,463 and 5,599,670) and green fluorescent protein (GFP) (Niedz et al., 1995, Plant Cell Rep., 14: 403).

One host cell that conveniently may be used is Escherichia coli. The preparation of the E. coli vectors may be accomplished using commonly known techniques such as restriction digestion, ligation, gel electrophoresis, DNA sequencing, the Polymerase Chain Reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13 mp series of vectors, pBluescript etc. Typically, these cloning vectors contain a marker allowing selection of transformed cells. Nucleic acid sequences may be introduced in these vectors, and the vectors may be introduced in E. coli by preparing competent cells, electroporation or using other well-known methodologies to a person of skill in the art. E. coli may be grown in an appropriate medium, such as Luria-Broth medium and harvested. As will be known to those of skill in the art, growth media may be adjusted depending on the host cell that is selected. Yeast cell media that may be used include yeast extract peptone dextrose (YPD) media. Animal cell media that may be used, for example, include Dulbecco Modified Eagle Medium (DMEM) or Opti-mem. Growth conditions, for example temperature, oxygenation, growth time etc. may be adjusted and optimized to achieve efficient host cell growth. These conditions, as will be recognized by those of skill in the art, depend on the host cell that is selected. Thus, for example, Escherichia coli cells may be grown for 12 - 24 hrs at about 37° C. in an incubator shaker that allows continuous stirring of the cells. It is further noted that in accordance with the present disclosure UDP-glycosylated compounds must be supplied. In general, UDP-glycosylated compounds are synthesized by the host cells as part of ordinary cellular metabolism, however if desired, UDP-glycosylated compounds may also be exogenously added to the cellular growth medium. Further, general guidance with respect to the preparation of recombinant vectors and growth of recombinant organisms may be found in, for example: Sambrook et al., Molecular Cloning, a Laboratory Manual, Cold Spring Harbor Laboratory Press, 2012, Fourth Ed.

Growth of the host cells can lead to expression of the UDP glycosyl transferase and enzymes in the cannabinoid biosynthetic enzyme complement, and, unexpectedly, to production of glycosylated cannabinoid compounds and, additionally, glycosylated cannabinoid precursor compounds.

In some embodiments, the glycosylation reaction may take place in the cytosolic compartment of the host cell.

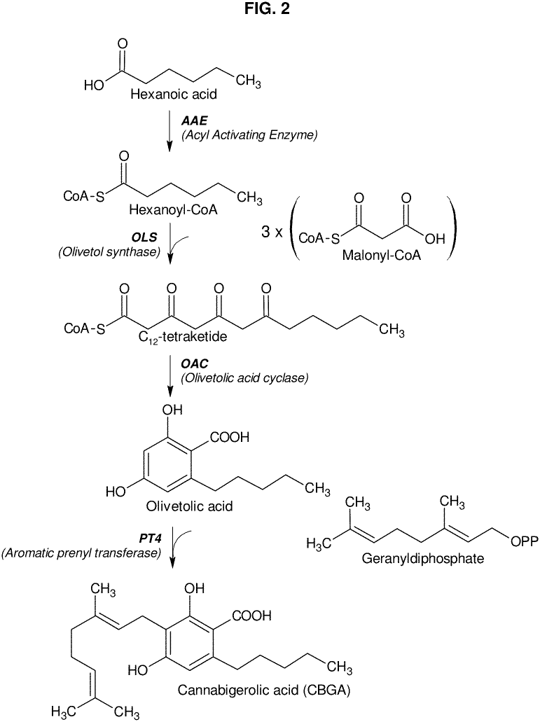

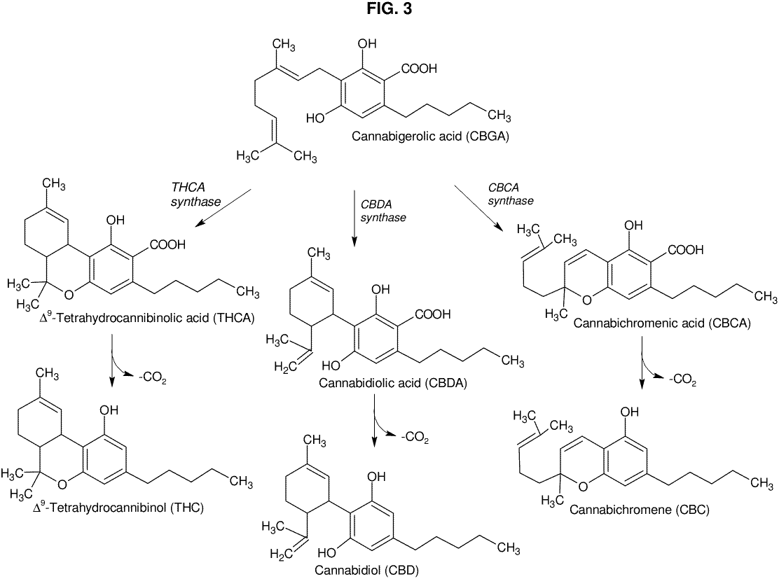

FIG. 2 depicts an exemplary biosynthetic pathway for the conversion of a cannabinoid precursor compound, notably hexanoic acid, hexanoyl-CoA, C₁₂-tetraketide and olivetolic acid to form the exemplary cannabinoid compound, cannabigerolic acid (CBGA). FIG. 3 depicts exemplary extensions of the biosynthetic pathway shown in FIG. 2 to provide the exemplary cannabinoid compounds, cannabidiolic acid (CBDA), Δ⁹-tetrahydrocannabinolic acid (Δ⁹-THCA), or cannabichromenic acid (CBCA). The conversion reactions depicted in the pathways of FIGS. 2 and 3 are catalyzed by various enzymes, including acyl activating enzyme (AAE), olivetol synthase, (OLS), olivetolic acid cyclase (OAC), prenyl transferase (PT), cannabidiolic acid synthase (CBDAS), Δ⁹-tetrahydrocannabinolic acid synthase (THCAS), or cannabichromenic acid synthase (CBCAS), which can be included in the host cell’s cannabinoid biosynthetic enzyme complement. It is noted that the conversion reaction from olivetolic acid to cannabigerolic acid (CBGA) requires the presence of geranyl pyrophosphate (GPP). GPP can be synthesized in the process of ordinary glycolysis by many host cells during cell growth, or alternatively GPP can be exogenously included in the host cell growth medium. In other embodiments, the conversion reaction may be performed using farnesyl pyrophosphate (FPP) in addition to, or instead of GPP.

Although FIG. 1 depicts a single UGT catalyzed glycosylation of the cannabinoid, CBGA, it is contemplated in the in vivo methods of the present disclosure that more than one glycosylated cannabinoid precursor and/or glycosylated cannabinoid compound can be formed by the recombinant host cell, more or less simultaneously. Thus, for example, in accordance with the present disclosure, in a cultured host cell glycosylated olivetolic acid may be formed by glycosylation of olivetolic acid in a reaction catalyzed by UDP glycosyl transferase, and glycosylated cannabigerolic acid (CBGA) may be formed in a reaction catalyzed by UDP glycosyl transferase. By way of another example, in accordance with the present disclosure, in a cultured cell glycosylated cannabigerolic acid (CBGA) may be formed and glycosylated cannabidiolic acid (CBCA) may be formed. Accordingly, it is contemplated that the culture medium produced by such a recombinant host cell is a composition comprising a mixture of the glycosylated cannabinoid precursor and glycosylated cannabinoid compounds described herein, e.g., a composition comprising a mixture of compounds selected from the compounds of structural formulas (I), (Ia), (Ib), (II), (IIa), (IIb), (III), (IIIa), (IV), (IVa), and combinations thereof.

Upon production by the host cells of the glycosylated cannabinoid compounds in accordance with the methods of the present disclosure, the glycosylated cannabinoid compounds may be extracted from the host cell suspension and separated from other constituents within the host cell suspension, such as media constituents and cellular debris. Separation techniques will be known to those of skill in the art and include, for example, solvent extraction (e.g., butane, chloroform, ethanol), column chromatography-based techniques, high-performance liquid chromatography (HPLC), for example, and/or countercurrent separation (CCS) based systems. The recovered glycosylated cannabinoid compounds may be obtained in a more or less pure form, for example, a preparation of halogenated cannabinoid compounds of at least about 60% (w/v), about 70% (w/v), about 80% (w/v), about 90% (w/v), about 95% (w/v) or about 99% (w/v) purity may be obtained.

In another aspect, the present disclosure provides, in at least one embodiment, a glycosylated cannabinoid compound produced in accordance with any one of the methods of the present disclosure.

It will be clear from the foregoing that the methods of the present disclosure may be used to make a variety of glycosylated cannabinoid compounds. The obtained glycosylated cannabinoid compounds may be formulated for use as a pharmaceutical drug, recreational drug, therapeutic agent or medicinal agent. Thus, the present disclosure further includes a pharmaceutical drug composition and a recreational drug composition comprising a glycosylated cannabinoid compound prepared in accordance with the methods of the present disclosure. Pharmaceutical and recreational drug preparations comprising a halogenated cannabinoid compound in accordance with the present disclosure can comprise vehicles, excipients and auxiliary substances, such as wetting or emulsifying agents, pH buffering substances and the like. Where pharmaceutical drug formulations are prepared, these vehicles, excipients and auxiliary substances are generally pharmaceutically acceptable agents that may be administered without undue toxicity. Pharmaceutically acceptable excipients include, but are not limited to, liquids such as water, saline, polyethylene glycol, hyaluronic acid, glycerol and ethanol. Pharmaceutically acceptable salts can also be included therein, for example, mineral acid salts such as hydrochlorides, phosphates, sulfates, and the like; and the salts of organic acids such as acetates, propionates, benzoates, and the like. It is also preferred, although not required, that the preparation will contain a pharmaceutically acceptable excipient that serves as a stabilizer. Examples of suitable carriers that also act as stabilizers include, without limitation, pharmaceutical grades of dextrose, sucrose, lactose, sorbitol, inositol, dextran, and the like. Other suitable carriers include, again without limitation, starch, cellulose, sodium or calcium phosphates, citric acid, glycine, polyethylene glycols (PEGs), and combinations thereof.

The pharmaceutical or recreational drug composition may be formulated for oral administration or for inhalation, or other routes of administration as desired. Dosing may vary and may be optimized, if desired, using routine experimentation.

Thus, in another aspect, the present disclosure provides, in at least one embodiment, a pharmaceutical drug composition or a recreational drug composition comprising a glycosylated cannabinoid compound produced in accordance with any one of the methods of the present disclosure.

In some embodiments, the recreational drug composition is a beverage.

In some embodiments, the recreational drug composition is a food product.

The glycosylated cannabinoid compounds of the present disclosure further may be used as precursor or feedstock material for the production of derivative cannabinoid compounds. Thus, for example, as has been described herein, cannabigerolic acid made in accordance the disclosure can be used as a precursor to make Δ⁹-tetrahydrocannabinolic acid. It will be clear to those of skill in the art that the glycosylated cannabinoid compounds made in accordance with the present disclosure can be used to make a wide variety of derivative glycosylated cannabinoid compounds. Upon finishing synthesis, the halogenated cannabinoid compounds can be used to formulate pharmaceutical drugs or recreational drugs, as hereinbefore described.

In yet further embodiments, the present disclosure provides methods for treating a patient with a pharmaceutical composition comprising a glycosylated cannabinoid compound prepared in accordance with the present disclosure. Accordingly, the present disclosure further provides a method for treating a patient with a glycosylated cannabinoid compound prepared according to the methods of the present disclosure, the method comprising administering to the patient a pharmaceutical composition comprising a glycosylated cannabinoid compound, wherein the pharmaceutical composition is administered in an amount sufficient to ameliorate a medical condition in the patient.

Hereinafter are provided examples of specific implementations for performing the methods of the present disclosure, as well as implementations representing the compositions of the present disclosure. The examples are provided for illustrative purposes only and are not intended to limit the scope of the present disclosure in any way.

### EXAMPLES

### Example 1: Expression of UDP-Glycosyl Transferases in Recombinant Yeast Cells With a Cannabinoid Producing Pathway

This example illustrates transformation of recombinant yeast cells, that are already engineered with a pathway capable of producing cannabinoids (e.g., CBGA) and cannabinoid precursors (e.g., olivetolic acid), with heterologous genes that express UGTs from Arabidopsis and Helianthus annuus.

### Materials and Methods

cDNAs encoding the following UGTs from Arabidopsis thaliana were cloned into pDONOR-zeo and recombined to the yeast expression vector pAG425GPD: AtUGT73C6 (SEQ ID NO: 4), AtUGT88A1 (SEQ ID NO: 6), AtUGT71 D1 (SEQ ID NO: 8), AtUGT73B4 (SEQ ID NO: 10), AtUGT76C4 (SEQ ID NO: 12), AtUGT76E12 (SEQ ID NO: 13), and At5g49690 (SEQ ID NO: 18).

The cDNAs encoding the UGTs derived from Cannabis sativa (CsUGT73C6; SEQ ID NO: 16) and Helianthus annuus (HaUGT76G1L; SEQ ID NO: 2) were also cloned into pDONOR-zeo and recombined to the yeast expression vector pAG425GPD.

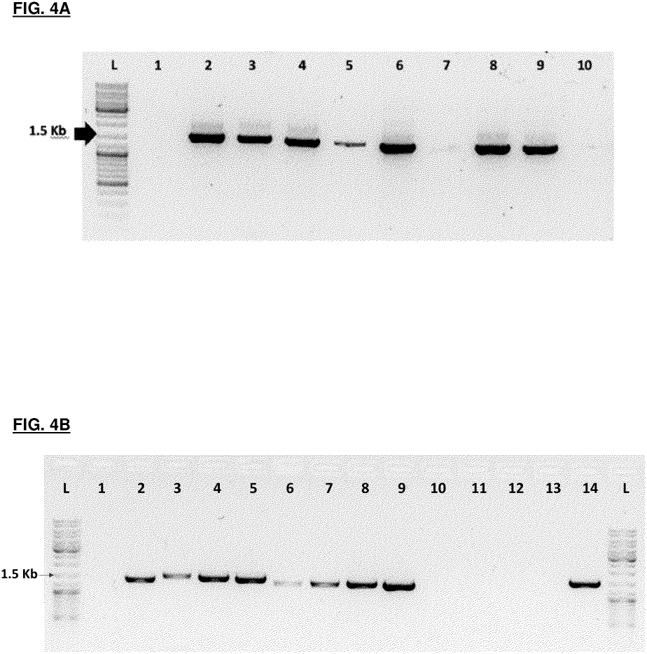

A recombinant yeast strain which includes a pathway capable of converting hexanoic acid to olivetolic acid, CBGA, and CBDA was transformed individually with the pAG425GPD vector constructs of the above noted UGT genes derived Arabidopsis thaliana, Cannabis sativa and Helianthus annuus. A total of 1 mL of 24-hour cultured yeast cells was harvested by centrifugation and total RNA was extracted using the RNeasy mini kit (Qiagen). To eliminate genomic DNA contamination, an additional DNase treatment was performed according to the DNasel protocol (Invitrogen). The extracted RNA was quantified using the EPOCH|2 microplate reader (BioTek). Quality and integrity were checked using 1.2 % agarose gel electrophoresis, images of which are depicted in FIGS. 4A and 4B. One microgram of total RNA was reverse transcribed into cDNA in a 20 µL reaction mixture using OneScript Plus cDNA synthesis kit (ABM). The transcribed cDNA was used to check for the expression of the transgenes by RT-PCR. Primers used for RT-PCR are listed in Table 3 below (see: SEQ ID NO: 41-79).

TABLE 3

| UGT | Forward Primer | SEQ ID NO: | Reverse Primer | SEQ ID NO: |
|---|---|---|---|---|
| AtUGT73C6 | ATGGCTTTCGAAAAAAACA | 41 | TCAATTATTGGACTGTGCTAG | 42 |
| AtUGT73B4 | ATGAACAGAGAGCAAATTC | 43 | CTACTTTCTACCATTCAGCTC | 44 |
| AtUGT71 D1 | ATGCGGAATGTAGAGCTCATC | 45 | CTAGGGCTTAATTCCTATCACG | 46 |
| HaUGT76G1L | ATGGAGACCCAAACAGAAA | 47 | CTAAAAGGAAGAAATATAAGCAA CT | 48 |
| AtUGT76E12 | ATGCAGGTTTTGGGAATG | 49 | TCATAGAGTCCTTATGAAGTGTA C | 50 |
| AtUGT88A1 | ATGGGTGAAGAAGCTATAGTTC TG | 51 | TCACTTTGGGCTCCACGA | 52 |
| At5g49690 | ATGGTCGACAAGAGAGAAGAAG | 53 | TCATGATGATGATGATGATCCT | 54 |
| AtUGT76C4 | ATGGAGAAGAGTAATGGC | 55 | CTAGAAAGATGATATATAATTAA TC | 56 |
| StUGT76G1 | ATGGAAAATAAAACGGAGACC | 57 | TTACAACGATGAAATGTAAGAAA CT | 58 |
| AtUGT85A3 | ATGGGATCCCGTTTTGTT | 59 | TTACGTGTTAGGGATCTTTCC | 60 |
| AtUGT79B1 | ATGGGTGTTTTTGGATCG | 61 | TCATGACTTCACAAGTTCAATTA A | 62 |
| At5g65550 | ATGGCCGAGCCAAAACCG | 63 | CTACACTCCTGCTATAGGATTCT CCA | 64 |
| AtUGT76B1 | ATGGAGACTAGAGAAACAA | 65 | TTAGAAAGACAATATATAAGCA | 66 |
| AtUGT76D1 | ATGGCAGAGATTCGCCAG | 67 | TCATTGTTCGTCAATTTGCATC | 68 |
| CsUGT75B2 | ATGGTTCAGCCAAGATTCTTGA | 69 | TCATTGGTGGATGGACTTCAC | 70 |
| CsUGT73B4 | ATGTCCAAGGGTCATACCATT | 71 | TCAGTAGTGATGGATTCTTTGC | 72 |
| CsUGT73B1 | ATGTCCAAAGAAATCTGGGTTG | 73 | TTACGTGAAATTGTTGATCATAT C | 74 |
| CsUGT71D1_ DN11028 | ATGAAGAGGACCTTGTTGTTTATT | 75 | TTACTTTTCTTCCACCACCAGC | 76 |
| CsUGT71D1_ DN4828 | ATGGCCAGAGTCGAATTGG | 77 | TCAGTAGTAATTGGAGCCGAT | 78 |
| CsUGT73C6 | ATGGCCTCTGAACCATACAAAT | 79 | TCAAGAGATGTGCAAGTTTCTG | 80 |

Results: As shown by the gel images depicted in FIGS. 4A and 4B, the host yeast cells transformed with the UGT vector constructs expressed most of the UDP-glycosyl transferases derived from Arabidopsis thaliana, Helianthus annuus, and Cannabis sativa and Stevia rebaudiana. AtUBG88A1 (lane 7, FIG. 4A), although not visually apparent in the gel image, exhibited activity indicating its expression as described in Example 2.

### Example 2: Detection of Glycosylated Cannabinoid Precursor Compounds and Glycosylated Cannabinoid Compounds in Yeast Cells Expressing UDP-Glycosyl Transferase

This example illustrates the fermentative production of glycosylated cannabinoid and glycosylated cannabinoid precursor compounds from recombinant yeast engineered with cannabinoid producing pathway and further transformed with UGT expressing genes from Arabidopsis thaliana, Helianthus annuus, and Cannabis sativa.

### Materials and Methods

CN3 yeast strain host cells were transformed as described in Example 1 with one of the following heterologous UGT genes: (1) AtUGT73C6, (2) AtUGT73B4, (3) AtUGT71 D1, (4) AtUGT76E12, (5) AtUGT88A1, (6) HaUGT76G1-L, (7) At5g49690, (8) AtUGT76C4, (9) CsUGT73C6, (10) SrUGT76C1, (11) AtUGT85A3, (12) AtUGT73B1, (13) Atg65550, (14) AtUGT76B1, (15) ATUGT76B1, (16) CsUGT75B2, (17) CsUGT73B4, (18) CsUGT73B1, (19) CsUGT75D1-DN11028, and (20) CsUGT71D1-DN48028. The transformed host cells were pre-grown overnight in yeast extract peptone dextrose (YPD) growth medium and then back diluted into yeast extract peptone galactose (YPG) to OD₆₀₀ = 0.2. Growth medium was supplemented with 0.2 mM hexanoic acid or 0.5 g/L CBD. Strains were incubated for 20 h at 28° C. rotating at 600 RPM in an EPOCH|2 microplate reader (BioTek). Subsequently, samples were treated with an extraction solvent (80 % Acetonitrile, 20 % Methanol) for 1 hour rotating at 100 RPM. After 20 minutes centrifugation at 12,000 RPM, the supernatant was filtered with a basix 13 mm syringe filter (0.22 µm pore size, Nylon membrane) and transferred to a new tube for further analysis.

Glycosylated cannabinoid compounds and glycosylated cannabinoid precursor compounds were assayed in the supernatant and the cellular pellet employing HPLC and HPLC-MS analysis. HPLC and HPLC-MS analysis was carried out as described below to detect the following glycosylated cannabinoid and cannabinoid precursor compounds: CBGA monoglucoside, CBGA diglucoside, CBDA monoglucoside, CBDA diglucoside, CBGA glucuronic acid, CBD monoglucoside, CBD diglucoside, olivetolic acid monoglucoside (“OliAcid monoglucoside”), olivetolic acid diglucoside (“OliAcid diglucoside”).

HPLC analysis was carried out on an Agilent Technologies 1290 Infinity system, consisting of a vacuum degasser, a binary pump, a thermostated autosampler, a thermostated column compartment and a diode array detector (DAD). A Zorbax Eclipse Plus EC-18 column (2.1 × 50 mm, 1.8 µm, Agilent, USA) was used with a mobile phase composed of 0.1% formic acid in both (A) water with 0.2 % Formic Acid and (B) Acetonitrile with 0.2 % Formic Acid. The chromatographic conditions were set as follows: 0.0-8.0 min linear gradient from 5 to 95% B; 8.1-9.09 min from 5 to 95% B, 9.10-11.0 min 5 to 95% A for equilibration of the column with the initial conditions. The flow rate was set at 0.4 ml/min. The column temperature was set at 40° C. The sample injection volume was 5 µL. The UV/DAD acquisitions were carried out in the range 190-400 nm and chromatograms were acquired at 265 and 350 nm.

HPLC-MS analysis was carried out to confirm the identity of the HPLC peaks using an Agilent Technologies 6530 Accurate-Mass quadrupole time of flight (QToF) mass spectrometer operating in negative ionization (ESI -) mode. The mass spectrometer experimental parameters were set as follows: the capillary voltage was 3.5 kV, the nebulizer (N₂) pressure was 35 psi, the drying gas temperature was 350° C., the drying gas flow was 11 L/min and the skimmer voltage was 65 V. Data were acquired by Agilent Mass Hunter software. The mass spectrometer was operated in full-scan mode in the m/z range 50-1100. Extracted ion chromatograms (EICs) were obtained with an accuracy of 10 ppm m/z from total ion chromatogram (TIC) employing the m/z corresponding to the molecular ions [M-H]⁻ 385.1504 for Olivetolic Acid Mono-Glucoside, 547.2032 for Olivetolic Acid di-Glucoside, 521.2756 for CBGA Mono-Glucoside, 683.3284 for CBGA Di-Glucoside, 535.2549 for CBGA Glucuronic Acid, 519.2600 for CBDA Mono-Glucoside, 475.2701 for CBD Mono-Glucoside, 637.3302 for CBD Di-Glucoside.

Results: HPLC-MS analysis results are summarized in Table 4 (below). The glycosylated cannabinoid compounds and glycosylated cannabinoid precursor compounds were detected in a relative and semi-quantitative fashion. If detected, relative semi-quantitative values of (+), (++), (+++), (++++) or (+++++), were assigned to express the detected quantity, wherein (+) represents the lowest detected quantities of a glycosylated cannabinoid compound or glycosylated cannabinoid precursor compound, and (+++++) represents the highest detected quantities. As will be understood, (++), (+++), and (++++) signify relative increasing intermediate detected levels of a glycosylated cannabinoid compound or glycosylated cannabinoid precursor compound. No detectable levels of a glycosylated cannabinoid compound or glycosylated cannabinoid precursor compound are indicated by “n.d.” and where compounds were not tested for is indicated by “N.T.”.

TABLE 4

| UGT (aa sequence) | OLA-glc | OLA-(glc)₂ | CBGA-glc | CBGA-(glc)₂ | CBDA-glc | CBGA-glucuronic acid | CBD-glc | CBD-(glc)₂ |
|---|---|---|---|---|---|---|---|---|
| | 20 h growth, detected in PELLET | | | | | | | |
| | 0.05 mM hexanoic acid | | | | | | 0.5 g/L CBD | |
| Negative Control | n.d. | n.d. | n.d. | n.d. | n.d. | n.d. | n.d. | n.d. |
| AtUGT73C6 (SEQ ID NO: 4) | +++ | ++ | ++++ | ++ | +++ | ++ | +++ | ++ |
| AtUGT73B4 (SEQ ID NO: 10) | ++++ | ++ | ++++ | ++ | n.d. | ++ | ++ | n.d. |
| AtUGT71 D1 (SEQ ID NO: 8) | ++ | n.d. | + | n.d. | ++ | n.d. | +++ | n.d. |
| AtUGT76E12 (SEQ ID NO: 14) | +++ | n.d. | +++ | n.d. | n.d. | n.d. | n.d. | n.d. |
| AtUGT88A1 (SEQ ID NO: 6) | + | n.d. | ++ | n.d. | n.d. | n.d. | n.d. | n.d. |
| HaUGT76G1L (SEQ ID NO: 2) | ++ | n.d. | n.d. | n.d. | ++ | n.d. | N.T. | N.T. |
| At5g49690 (SEQ ID NO: 18) | | n.d. | n.d. | n.d. | + | n.d. | N.T. | N.T. |
| AtUGT76C4 (SEQ ID NO: 12) | ++ | n.d. | n.d. | n.d. | n.d. | n.d. | n.d. | n.d. |
| CsUGT73C6 (SEQ ID NO: 16) | ++ | n.d. | n.d. | n.d. | n.d. | n.d. | n.d. | n.d. |
| SrUGT76G1 (SEQ ID NO: 20) | n.d. | n.d. | n.d. | n.d. | n.d. | n.d. | n.d. | n.d. |
| AtUGT85A3 (SEQ ID NO: 22) | n.d. | n.d. | n.d. | n.d. | n.d. | n.d. | n.d. | n.d. |
| AtUGT73B1 (SEQ ID NO: 24) | N.T. | N.T. | N.T. | N.T. | N.T. | N.T. | n.d. | n.d. |
| At5g65550 (SEQ ID NO: 26) | n.d. | n.d. | n.d. | n.d. | n.d. | n.d. | N.T. | N.T. |
| AtUGT76B1 (SEQ ID NO: 28) | N.T. | N.T. | N.T. | N.T. | N.T. | N.T. | n.d. | n.d. |
| AtUGT76D1 (SEQ ID NO: 30) | N.T. | N.T. | N.T. | N.T. | N.T. | N.T. | n.d. | n.d. |
| CsUGT75B2 (SEQ ID NO: 32) | n.d. | n.d. | n.d. | n.d. | n.d. | n.d. | N.T. | N.T. |
| CsUGT73B4 (SEQ ID NO: 34) | n.d. | n.d. | n.d. | n.d. | n.d. | n.d. | N.T. | N.T. |
| CsUGT73B1 (SEQ ID NO: 36) | n.d. | n.d. | n.d. | n.d. | n.d. | n.d. | N.T. | N.T. |
| CsUGT75D1-DN11028 (SEQ ID NO: 38) | n.d. | n.d. | n.d. | n.d. | n.d. | n.d. | N.T. | N.T. |
| CsUGT71 D1-DN48028 (SEQ ID NO: 40) | n.d. | n.d. | n.d. | n.d. | n.d. | n.d. | N.T. | N.T. |
| UGT (aa sequence) | 20 h growth, detected in SUPERNATANT | | | | | | | |
| | 0.2 mM hexanoic acid | | | | | | 0.5 g/L CBD | |
| Negative Control | n.d. | n.d. | n.d. | n.d. | n.d. | n.d. | n.d. | n.d. |
| AtUGT73C6 (SEQ ID NO: 4) | +++ | n.d. | + | + | n.d. | n.d. | +++ | ++ |
| AtUGT73B4 (SEQ ID NO: 10) | +++++ | ++ | ++ | n.d. | n.d. | n.d. | ++ | n.d. |
| AtUGT71 D1 (SEQ ID NO: 8) | ++ | n.d. | n.d. | n.d. | n.d. | n.d. | +++ | n.d. |
| AtUGT76E12 (SEQ ID NO: 14) | +++ | n.d. | + | n.d. | n.d. | n.d. | N.T. | N.T. |
| AtUGT88A1 (SEQ ID NO: 6) | n.d. | n.d. | n.d. | n.d. | n.d. | n.d. | n.d. | n.d. |
| HaUGT76G1L (SEQ ID NO: 2) | n.d. | n.d. | n.d. | n.d. | N.T. | n.d. | n.d. | n.d. |
| At5g49690 (SEQ ID NO: 18) | n.d. | n.d. | n.d. | n.d. | n.d. | n.d. | n.d. | n.d. |
| AtUGT76C4 (SEQ ID NO: 12) | n.d. | n.d. | n.d. | n.d. | n.d. | n.d. | n.d. | n.d. |
| CsUGT73C6 (SEQ ID NO: 16) | ++ | n.d. | n.d. | n.d. | n.d. | n.d. | N.T. | N.T. |
| SrUGT76G1 (SEQ ID NO: 20) | n.d. | n.d. | n.d. | n.d. | n.d. | n.d. | n.d. | n.d. |
| AtUGT85A3 (SEQ ID NO: 22) | n.d. | n.d. | n.d. | n.d. | n.d. | n.d. | n.d. | n.d. |
| AtUGT73B1 (SEQ ID NO: 24) | N.T. | N.T. | N.T. | N.T. | N.T. | N.T. | n.d. | n.d. |
| At5g65550 (SEQ ID NO: 26) | n.d. | n.d. | n.d. | n.d. | n.d. | n.d. | N.T. | N.T. |
| AtUGT76B1 (SEQ ID NO: 28) | N.T. | N.T. | N.T. | N.T. | N.T. | N.T. | n.d. | n.d. |
| AtUGT76D1 (SEQ ID NO: 30) | N.T. | N.T. | N.T. | N.T. | N.T. | N.T. | n.d. | n.d. |
| CsUGT75B2 (SEQ ID NO: 32) | n.d. | n.d. | n.d. | n.d. | n.d. | n.d. | N.T. | N.T. |
| CsUGT73B4 (SEQ ID NO: 34) | n.d. | n.d. | n.d. | n.d. | n.d. | n.d. | N.T. | N.T. |
| CsUGT73B1 (SEQ ID NO: 36) | n.d. | n.d. | n.d. | n.d. | n.d. | n.d. | N.T. | N.T. |
| CsUGT75D1-DN11028 (SEQ ID NO: 38) | n.d. | n.d. | n.d. | n.d. | n.d. | n.d. | N.T. | N.T. |
| CsUGT71 D1-DN48028 (SEQ ID NO: 40) | n.d. | n.d. | n.d. | n.d. | n.d. | n.d. | N.T. | N.T. |

The production of glycosylated cannabinoids or cannabinoid precursor compounds was detected from recombinant yeast host cells transformed with the following UGTs from Arabidopsis thaliana: AtUGT73C6 (SEQ ID NO: 4), AtUGT88A1 (SEQ ID NO: 6), AtUGT71 D1, (SEQ ID NO: 8), AtUGT73B4 (SEQ ID NO: 10), AtUGT76C4 (SEQ ID NO: 12), AtUGT76E12 (SEQ ID NO: 14), At5g49690 (SEQ ID NO: 18). The glycosylated cannabinoids detected at various levels in both pelleted cells and the growth medium supernatant were: CBGA monoglucoside (“CBGA-glc”), CBGA diglucoside (“CBGA-(glc)₂”), CBDA monoglucoside (“CBDA-glc”), CBDA diglucoside (“CBDA-(glc)₂”), CBGA glucuronic acid, CBD monoglucoside (“CBD-glc”) and CBD diglucoside (“CBD-(glc)₂”).

The production of a glycosylated cannabinoid, CBDA-glc, and the glycosylated cannabinoid precursor compound, OLA-glc, was detected in the pellet from recombinant yeast host cells transformed with the UGT from Helianthus annuus, HaUGT76G1L. Only the production of the glycosylated cannabinoid precursor, OLA-glc, was detectable in the pellet or supernatant of recombinant yeast host cells transformed with the UGT from Cannabis sativa, CsUGT73C6.

No production of glycosylated cannabinoids or cannabinoid precursor compounds was detected from the pellet or supernatant of recombinant yeast host cells transformed with the following UGTs from Stevia rebaudiana, Cannabis sativa, and Arabidopsis thaliana: SrUGT76G1 (SEQ ID NO: 20), AtUGT85A3 (SEQ ID NO: 22), AtUGT73B1 (SEQ ID NO: 24), At5g65550 (SEQ ID NO: 26), AtUGT76B1 (SEQ ID NO: 28), AtUGT76D1 (SEQ ID NO: 30), CsUGT75B2 (SEQ ID NO: 32), CsUGT73B4 (SEQ ID NO: 34), CsUGT73B1 (SEQ ID NO: 36), CsUGT75D1-DN11028 (SEQ ID NO: 38), CsUGT71D1-DN48028 (SEQ ID NO: 40).

### Example 3: Production of Glycosylated Cannabinoid and Glycosylated Cannabinoid Precursor Compounds in Prokaryotic Cells Expressing Heterologous UGTs

This example illustrates the fermentative production of glycosylated cannabinoid and glycosylated cannabinoid precursor compounds from recombinant yeast engineered with cannabinoid producing pathway and further transformed with UGT expressing genes from Arabidopsis thaliana, Helianthus annuus, and Cannabis sativa.

### Materials and Methods

The following cDNAs encoding UGTs from Arabidopsis thaliana, Cannabis sativa and Helianthus annuus UGTs were cloned into pDONOR-zeo (as described in Example 1) and then recombined into the prokaryotic expression vector pDEST14: AtUGT73C6 (SEQ ID NO: 3), AtUGT88A1 (SEQ ID NO: 5), AtUGT71 D1 (SEQ ID NO: 7), AtUGT73B4 (SEQ ID NO: 9), AtUGT76C4 (SEQ ID NO: 11), AtUGT76E12 (SEQ ID NO: 13), and At5g49690 (SEQ ID NO: 17), CsUGT73C6 (SEQ ID NO: 15), HaUGT76G1L (SEQ ID NO: 1), and SrUGT76G1 (SEQ ID NO: 19). Host cells from the bacterial strain BL21 (DE3) were transformed individually pDEST14 vector.

A BL21 (DE3) single colony was inoculated in liquid media and incubated at 37° C. overnight. The bacterial cultures were diluted to a final 0.6 OD and CBDA was added to a final concentration of 0.1 mM. The cultures were split, and half was induced with 100 µM IPTG for 4h at 37° C. to express the UGTs and the other half was kept as controls without induction of UGT expression.

Subsequently, samples were treated with a 1:1 volume of acetonitrile for 15 minutes at 250 RPM. After 30 minutes centrifugation at 4,000 RPM, samples were diluted 1000-fold in the same solvent for further analysis.

CBDA depletion was assayed employing UHPLC-MS analysis. The instrument used was a Thermo Vanquish UHPLC connected to a Thermo TSQ Altis mass spectrometer. The UHPLC consists of a vacuum degasser, a ternary pump, a thermostated autosampler held at 5° C., and a thermostated column compartment. An Accucore C18 (150 × 2.2 mm, 2.6 µm, Thermo, USA) was used. The mobile phase is water with 0.1 % formic acid (A) and acetonitrile with 0.1 % formic acid (B) on a linear gradient (see Table 5). The flow rate was set at 0.800 mL/min. The column temperature was set at 30° C. The sample injection volume was 1 µL.

TABLE 5

Gradient timetable

| Time (min) | % A | % B |
|---|---|---|
| 0.000 | 100.0 | 0.0 |
| 1.000 | 50.0 | 50.0 |
| 1.500 | 25.0 | 75.0 |
| 2.000 | 21.0 | 79.0 |
| 2.100 | 20.0 | 80.0 |
| 2.500 | 19.0 | 81.0 |
| 2.600 | 18.0 | 82.0 |
| 3.000 | 18.0 | 82.0 |
| 3.100 | 10.0 | 90.0 |
| 4.000 | 10.0 | 90.0 |
| 4.100 | 100.0 | 0.0 |
| 6.000 | 100.0 | 0.0 |

MS analyses were carried out in order to ensure the identity of the peaks and were performed on a Thermo TSQ Altis triple quadrupole mass spectrometer using electrospray ionization in negative mode. Compounds were analyzed using selected reaction monitoring using two ion pairs for quantitation and confirmation respectively. Settings are summarized in Tables 6 and 7.

TABLE 6

Global ion source parameters

| | |
|---|---|
| Capillary Voltage | 3500 V |
| Sheath Gas | 60 Arb |
| Aux Gas | 15 Arb |
| Sweep gas | 2 Arb |
| Ion Transfer Tube Temp | 380° C. |
| Vaporizer Temp | 350° C. |
| Dwell Time | 38.409 ms |

TABLE 7

SRM parameters

| Compound | Precursor Ion (m/z) | Product Ion (m/z) | Collision Energy (V) | RF Lens (V | |
|---|---|---|---|---|---|
| CBDA | 357.212 | 245.25 | 28.13 | 77 | Confirmation |
| | | 339.28 | 20.24 | 77 | Quantitative |
| | | 341.292 | 19 | 77 | Quantitative |

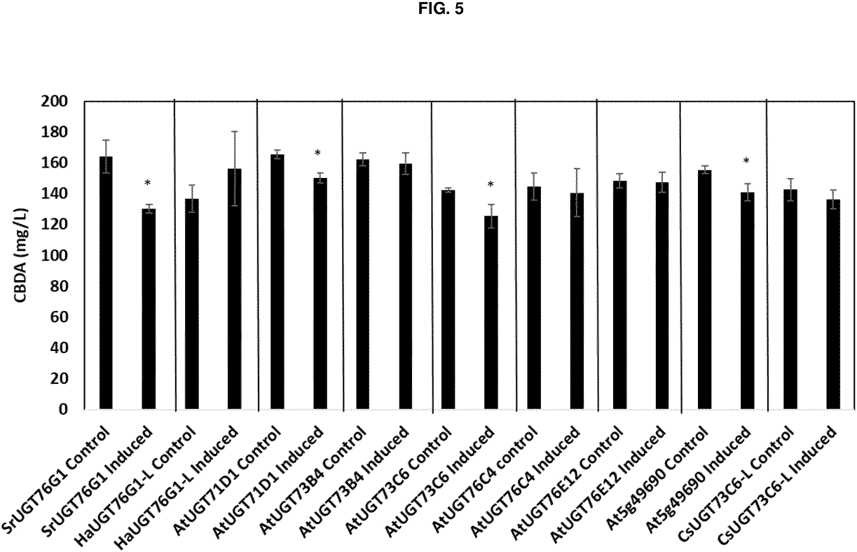

Results: As shown by the results plotted in FIG. 5, the bacterial strains carrying SrUGT76G1, AtUGT71D, AtUGT73C6 and At5g49690 genes showed statistically significant decreases in CBDA content (p ≤ 0.05). While SrUGT76G1 showed the highest decrease in CBDA content of 21%, AtUGT73C6 showed a decrease of 12% and AtUGT71 D1 and At5g49690 showed a 9% decrease in CBDA content. These results strongly suggest that the three UGTs from Arabidopsis thaliana are capable of producing a glycosylated CBDA when expressed in a prokaryotic cell system.

What is claimed is:

1. A recombinant host cell comprising:
(a) a pathway capable of producing a cannabinoid or a cannabinoid precursor; and
(b) a heterologous nucleic acid that encodes a UDP-glycosyl transferase derived from Arabidopsis thaliana or Helianthus annuus; wherein the host cell is capable of producing a glycosylated cannabinoid and/or a glycosylated cannabinoid precursor.

2. The host cell of claim 1, wherein the heterologous nucleic acid encodes an amino acid sequence having at least 90% identity to a sequence selected from SEQ ID NO: 4, 10, 8, 14, 2, 6, 16, or 18.

3. The host cell of claim 1, wherein the pathway capable of producing a cannabinoid or a cannabinoid precursor comprises:
(a) enzymes capable of converting hexanoic acid to olivetolic acid;
(b) an enzyme capable of converting olivetolic acid and geranyldiphosphate to CBGA;
(c) enzymes capable of catalyzing reactions (i) - (iii): 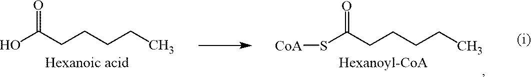 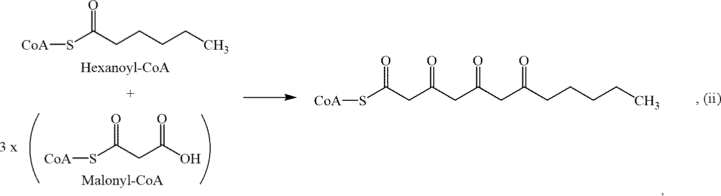 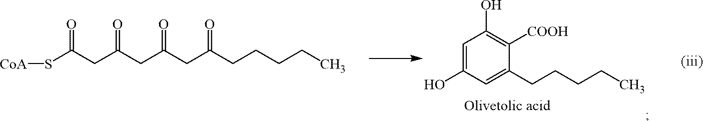
(d) an enzyme capable of catalyzing reaction (iv): 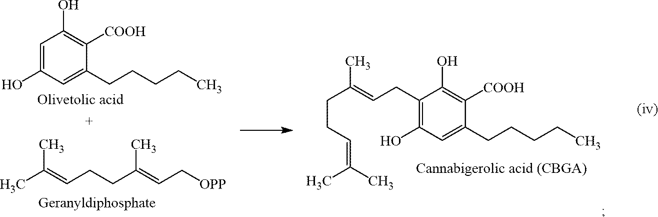
(e) the pathway comprises at least the following enzymes: AAE, OLS, and OAC; optionally, wherein the enzymes AAE, OLS, and OAC have an amino acid sequence of at least 90% identity to SEQ ID NO: 82 (AAE), SEQ ID NO: 84 (OLS), and SEQ ID NO: 86 (OAC), respectively; and/or
(f) the pathway comprises the enzyme PT4; optionally, wherein the enzyme PT4 has an amino acid sequence of at least 90% identity to SEQ ID NO: 88 or 90.

4. The host cell of claim 3, wherein the pathway further comprises an enzyme capable of catalyzing the conversion of CBGA to Δ⁹-THCA, CBDA, and/or CBCA;
optionally, wherein the pathway further comprises
(a) an enzyme capable of catalyzing a reaction (v), (vi), and/or (vii): 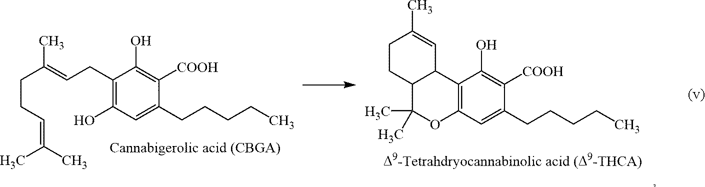 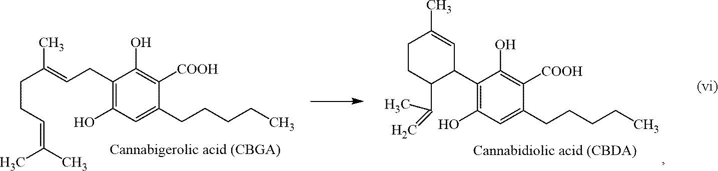 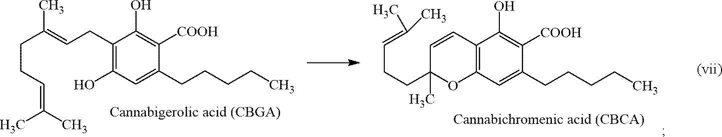
and/or
(b) a THCA synthase, a CBDA synthase, and/or a CBCA synthase; optionally, wherein the pathway comprises a CBDA synthase having an amino acid sequence of at least 90% identity to SEQ ID NO: 92 or 94.

5. The host cell of claim 1, wherein the host cell is a microbial cell; optionally, a cell derived from a source selected from: Saccharomyces cerevisiae, Escherichia coli, Yarrowia lipolytica, and Pichia pastoris.

6. The host cell of claim 1, wherein the cell is capable of producing a glycosylated cannabinoid, wherein:
(i) the glycosylated cannabinoid is a compound of structural formula (I): 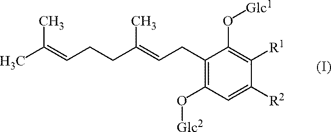
wherein, R¹ is H or COOH; R² is a C2-C7 alkyl chain; and at least one of Glc¹ and Glc² is a glycosyl group, and if either of Glc¹ or Glc² is not a glycosyl group then it is —H;
(ii) the glycosylated cannabinoid is a compound of structural formula (II): 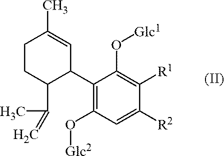
wherein, R¹ is H or COOH; R² is a C2-C7 alkyl chain; and at least one of Glc¹ and Glc² is a glycosyl group, and if either of Glc¹ or Glc² is not a glycosyl group then it is —H;
(iii) the glycosylated cannabinoid is a compound of structural formula (III): 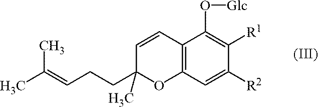
wherein, R¹ is H or COOH; R² is a C2-C7 alkyl chain; and Glc is a glycosyl group;
(iv) the glycosylated cannabinoid is a compound of structural formula (IV): 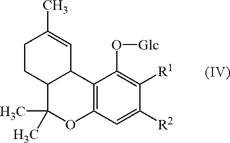
wherein, R¹ is H or COOH; R² is a C2-C7 alkyl chain; and Glc is a glycosyl group;
(v) the glycosylated cannabinoid precursor is a compound of structural formula (V): 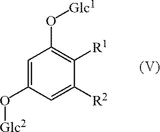
wherein, R¹ is H or COOH; R² is a C2-C7 alkyl chain; and at least one of Glc¹ and Glc² is a glycosyl group, and if either of Glc¹ or Glc² is not a glycosyl group then it is —H; or
(vi) the glycosylated cannabinoid or glycosylated cannabinoid precursor is selected from the compounds of structural formulas (Ia), (Ib), (IIa), (IIb), (IIIa), (IVa), (Va), and (Vb).

7. The host cell of claim 6, wherein:
(a) the glycosyl group, Glc, Glc¹, and/or Glc² is a moiety of structural formula (VI): 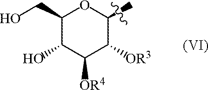
wherein
R³ is H, β-D-glucopyranosyl, or 3-O-β-D-glucopyranosyl-β-D-glucopyranosyl; and
R⁴ is H, β-D-glucopyranosyl, or 3-O-β-D-glucopyranosyl-β-D-glucopyranosyl;
(b) the glycosyl group Glc, Glc¹, and/or Glc² is a mono-saccharide, a di-saccharide, or a tri-saccharide;
(c) the glycosyl group Glc, Glc¹, and/or Glc² comprises a glucosyl group, a galactosyl group, a xylosyl group, a glucuronic acid group, an N-acetylglucosyl group, an N-acetylgalactosyl group, a fucosyl group, a mannosyl group, a sialic acid group, an arabinosyl group, a rhamnosyl group, or a combination thereof.

8. The host cell of claim 1, wherein the UDP-glycosyl substrate is selected from UDP-glucose, UDP-galactose, UDP-xylose, UDP-glucuronic acid, UDP-N-acetylglucosamine, UDP-N-acetylgalactosamine, GDP-fucose, GDP-mannose, CMP-sialic acid, and a mixture thereof.

9. A method for preparing a glycosylated cannabinoid and/or glycosylated cannabinoid precursor, the method comprising:
(a) culturing in a suitable medium a recombinant host cell of claim 1; and
(b) recovering the produced glycosylated cannabinoid, and/or glycosylated cannabinoid precursor.

10. The method of claim 9, wherein the method further comprises: (c) contacting a cell-free extract of the culture with a biocatalytic reagent or chemical reagent.

11. A method of producing a glycosylated cannabinoid or a glycosylated cannabinoid precursor, the method comprising contacting under suitable reaction conditions:
(a) a UDP-glycosyl transferase derived from Arabidopsis thaliana or Helianthus annuus;
(b) a UDP-glycosyl substrate comprising a glycosyl group; and
(c) a cannabinoid or a cannabinoid precursor comprising a hydroxyl group;
whereby the glycosyl group is transferred to the hydroxyl group to form the glycosylated cannabinoid or the glycosylated cannabinoid precursor.

12. The method of claim 11, wherein the UDP-glycosyl transferase comprises an amino acid sequence having at least 90% identity to a sequence selected from SEQ ID NO: 4, 10, 8, 14, 2, 6, 16, or 18.

13. The method of claim 11, wherein the cannabinoid or cannabinoid precursor comprises at least two hydroxyl groups.

14. The method of claim 11, wherein the cannabinoid precursor is a cannabinoid precursor selected from olivetolic acid, divarinic acid, 2-heptyl-4,6-dihydroxybenzoic acid, and 2-butyl-4,6-dihydroxybenzoic acid.

15. The method of 11, wherein the cannabinoid is selected from cannabigerolic acid (CBGA), cannabigerol (CBG), cannabidiolic acid (CBDA), cannabidiol (CBD), Δ9-tetrahydrocannabinolic acid (Δ9-THCA), Δ9-tetrahydrocannabinol (Δ9-THC), Δ8-tetrahydrocannabinolic acid (Δ8-THCA), Δ8-tetrahydrocannabinol (Δ8-THC), cannabichromenic acid (CBCA), cannabichromene (CBC), cannabinolic acid (CBNA), cannabinol (CBN), cannabidivarinic acid (CBDVA), cannabidivarin (CBDV), Δ9-tetrahydrocannabivarinic acid (Δ9-THCVA), Δ9-tetrahydrocannabivarin (Δ9-THCV), cannabidibutolic acid (CBDBA), cannabidibutol (CBDB), Δ9-tetrahydrocannabutolic acid (Δ9-THCBA), Δ9-tetrahydrocannabutol (Δ9-THCB), cannabidiphorolic acid (CBDPA), cannabidiphorol (CBDP), Δ9-tetrahydrocannabiphorolic acid (Δ9-THCPA), Δ9-tetrahydrocannabiphorol (Δ9-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), and cannabielsoin (CBE).

16. The method of claim 11, wherein:
(i) the glycosylated cannabinoid is a compound of structural formula (I): 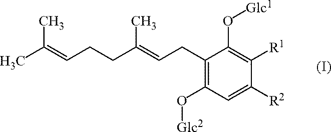
wherein, R¹ is H or COOH; R² is a C2-C7 alkyl chain; and at least one of Glc¹ and Glc² is a glycosyl group, and if either of Glc¹ or Glc² is not a glycosyl group then it is —H;
(ii) the glycosylated cannabinoid is a compound of structural formula (II): 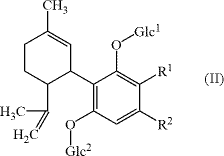
wherein, R¹ is H or COOH; R² is a C2-C7 alkyl chain; and at least one of Glc¹ and Glc² is a glycosyl group, and if either of Glc¹ or Glc² is not a glycosyl group then it is —H;
(iii) the glycosylated cannabinoid is a compound of structural formula (III): 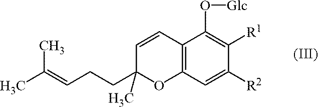
wherein, R¹ is H or COOH; R² is a C2-C7 alkyl chain; and Glc is a glycosyl group;
(iv) the glycosylated cannabinoid is a compound of structural formula (IV): 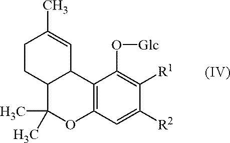
wherein, R¹ is H or COOH; R² is a C2-C7 alkyl chain; and Glc is a glycosyl group;
(v) the glycosylated cannabinoid precursor is a compound of structural formula (V): 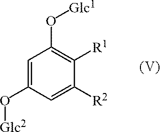
wherein, R¹ is H or COOH; R² is a C2-C7 alkyl chain; and at least one of Glc¹ and Glc² is a glycosyl group, and if either of Glc¹ or Glc² is not a glycosyl group then it is —H; or
(vi) the glycosylated cannabinoid or glycosylated cannabinoid precursor is selected from the compounds of structural formulas (Ia), (Ib), (IIa), (IIb), (IIIa), (IVa), (Va), and (Vb).

17. The method of claim 16, wherein:
(a) the glycosyl group, Glc, Glc¹, and/or Glc² is a moiety of structural formula (VI): 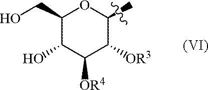
wherein
R³ is H, β-D-glucopyranosyl, or 3-O-β-D-glucopyranosyl-β-D-glucopyranosyl; and
R⁴ is H, β-D-glucopyranosyl, or 3-O-β-D-glucopyranosyl-β-D-glucopyranosyl;
(b) the glycosyl group Glc, Glc¹, and/or Glc² is a mono-saccharide, a di-saccharide, or a tri-saccharide;
(c) the glycosyl group Glc, Glc¹, and/or Glc² comprises a glucosyl group, a galactosyl group, a xylosyl group, a glucuronic acid group, an N-acetylglucosyl group, an N-acetylgalactosyl group, a fucosyl group, a mannosyl group, a sialic acid group, an arabinosyl group, a rhamnosyl group, or a combination thereof.

18. The method of claim 11, wherein the UDP-glycosyl substrate is selected from UDP-glucose, UDP-galactose, UDP-xylose, UDP-glucuronic acid, UDP-N-acetylglucosamine, UDP-N-acetylgalactosamine, GDP-fucose, GDP-mannose, CMP-sialic acid, and a mixture thereof.

19. The method of claim 11, wherein the contacting under suitable reaction conditions comprises of in vitro conditions.


SEQUENCE LISTING (94 sequences)

SEQ ID NO: 1 - DNA, 1392 bp, Helianthus annuus
  source 1..1392: mol_type = genomic DNA
atggagacccaaacagaaaccaccaacaccgttcgccggaaccagagaataatattcttcccgttaccatatcaaggccacataaacccaatgctccaacttgccaatctactctactccaaaggcttcagtatcaccatcctccacaccaacttcaacaagcccaaaacatccaactaccctcacttcactttcaaattcatcctggacaacgatccacacgacgaacgctattccaatctaccgttacatggcatgggcgcttttaaccgccttttcgtgttcaacgaagatggtgcagatgaattgcgccatgaacttgaactgttaatgttagcttcgaaagaagatgacgaacatgtatcgtgtttaatcaccgatgcgctttggcacttcacgcaatcagtcgctgacagccttaacctcccacggcttgttttgaggacaagcagcttgttttgttttcttgcttatgcttcatttcctgtttttgatgatcttggttaccttaatcttgctgatcaaacacgtctggacgaacaagtggctgagtttcctatgttgaaagtgagagatattataaagttgggctttaagagctcgaaagattctattggaatgatgcttggtaatatggtgaaacaaacgaaagcgtctttgggtattatctttaactcgtttaaggaactcgaagagccggaggttgaaactgttatccgtgatattctggcaccgagttttctgataccatttccaaagcatttcacagcgtcatccagcagcttactagaccaagatcgaaccgtttttccatggttagaccaacagccgcctaattccgttttgtatgttagttttggtagcacgactgaagtggatgagaaagatttcttggaaatagctcatgggttggttgatagcgagcagacgtttttatgggtggttcgacctgggtatgtcaagggtccgatttggatcgaactgttggatgatgggttcgttggtgaaaaggggcgtattgtgaaatgggctcctcagcaagaagtgctagctcatgaagcaataggtgcgttttggactcatagtggatggaactcgacattggaaagcgtttgtgaaggtgttcctatgataatgtcgccttttatgggcgatcaagcgttgaacgctagatacatgagtgatgtttccaaggtaggggtgtatttgggaaacgggtgggaaagacgagagatagcgagtgccataaggaaagtaatggtggatgaagaaggagaacacattagagagaatgcaagagatttgaaacaaaaggcagatgattctttagtgaagggtgggtcttcctatgagtcattagagtctctagttgcttatatttcttccttttag

SEQ ID NO: 2 - protein, 477 aa, Helianthus annuus
METQTETTNTVRRNRRIIFFPLPYQGHINPMLQLANLLYSKGFSITILHTNFNKPKTSNYPHFTFKFILDNDPHDGRYSNLPLHGMGAFNRVFVFNEDGADELRHELELLMLASKEDDEHVSCLITDALWHFTQSVADSLSLPRLVLRTSSLFSFLIYASIPLLDDRGYLSLSDNTMALILDGLGYHDLSDNKRLEEQVEEFPMLKVKDIVKMGFKREKDGAGGMIDNMVKQTKTSSGIIWNSFKELEESELETIRRDIPAPSFPIPFAKHFTASSSSLLEHDRSFFPWLDQQPPKSVVYVSFGSVAQVEEKHFMEMVHGLVDSKQLFLWVVRPGFVSGSTWLEPLPDGFPGERGRIVKWAPQQEVLGHEAIGAFWTHGGWNSTLESVCEGVPMICSPFWGDQPLDARYVSDVWKVGVYLENGWKREEITGAIRRVMTDEEMRERARVLKQKLDVSLMKGGSSYESVESLVAYVSSF

SEQ ID NO: 3 - DNA, 1488 bp, Arabidopsis thaliana
  source 1..1488: mol_type = genomic DNA
atggctttcgaaaaaaacaacgaaccttttcctcttcactttgttctcttccctttcatggctcaaggccacatgattcccatggttgatattgcaaggctcttggctcagcgaggtgtgcttataacaattgtcacgacgcctcacaatgcagcaaggttcaagaatgtcctaaaccgtgccattgagtctggtttgcccatcaacctagtgcaagtcaagtttccatatcaagaagctggtctgcaagaaggacaagaaaatatggatttgcttaccacgatggagcagataacatctttctttaaagcggttaacttactcaaagaaccagtccagaaccttattgaagagatgagcccgcgaccaagctgtctaatctctgatatgtgtttgtcgtatacaagcgaaatcgccaagaagttcaaaataccaaagatcctcttccatggcatgggttgcttttgtcttctgtgtgttaacgttctgcgcaagaaccgtgagatcttggacaatttaaagtctgataaggagtacttcattgttccttattttcctgatagagttgaattcacaagacctcaagttccggtggaaacatatgttcctgcaggctggaaagagatcttggaggatatggtagaagcggataagacatcttatggtgttatagtcaactcatttcaagagctcgaacctgcgtatgccaaagacttcaaggaggcaaggtctggtaaagcatggaccattggacctgtttccttgtgcaacaaggtaggagtagacaaagcagagaggggaaacaaatcagatattgatcaagatgagtgccttgaatggctcgattctaaggaaccgggatctgtgctctacgtttgccttggaagtatttgtaatcttcctctgtctcagctccttgagctgggactaggcctagaggaatcccaaagacctttcatctgggtcataagaggttgggagaaatacaaagagttagttgagtggttctcggaaagcggctttgaagatagaatccaagatagaggacttctcatcaaaggatggtcccctcaaatgcttatcctttcacatccttctgttggagggttcttaacgcactgcggatggaactcgactcttgaggggataactgctggtctaccaatgcttacatggccactatttgcagaccaattctgcaacgagaaactggtcgtacaaatactaaaagtcggtgtaagtgccgaggttaaagaggtcatgaaatggggagaagaagagaagataggagtgttggtggataaagaaggagtgaagaaggcagtggaagaactaatgggtgagagtgatgatgcaaaagagagaagaagaagagccaaagagcttggagaatcagctcacaaggctgtggaagaaggaggctcctctcattctaatatcactttcttgctacaagacataatgcaactagcacagtccaataattga

SEQ ID NO: 4 - protein, 495 aa, Arabidopsis thaliana
MAFEKNNEPFPLHFVLFPFMAQGHMIPMVDIARLLAQRGVLITIVTTPHNAARFKNVLNRAIESGLPINLVQVKFPYQEAGLQEGQENMDLLTTMEQITSFFKAVNLLKEPVQNLIEEMSPRPSCLISDMCLSYTSEIAKKFKIPKILFHGMGCFCLLCVNVLRKNREILDNLKSDKEYFIVPYFPDRVEFTRPQVPVETYVPAGWKEILEDMVEADKTSYGVIVNSFQELEPAYAKDFKEARSGKAWTIGPVSLCNKVGVDKAERGNKSDIDQDECLEWLDSKEPGSVLYVCLGSICNLPLSQLLELGLGLEESQRPFIWVIRGWEKYKELVEWFSESGFEDRIQDRGLLIKGWSPQMLILSHPSVGGFLTHCGWNSTLEGITAGLPMLTWPLFADQFCNEKLVVQILKVGVSAEVKEVMKWGEEEKIGVLVDKEGVKKAVEELMGESDDAKERRRRAKELGESAHKAVEEGGSSHSNITFLLQDIMQLAQSNN

SEQ ID NO: 5 - DNA, 1389 bp, Arabidopsis thaliana
  source 1..1389: mol_type = genomic DNA
atgggtgaagaagctatagttctgtatcctgcaccaccaataggtcacttagtgtccatggttgagttaggtaaaaccatcctctccaaaaacccatctctctccatccacattatcttagttccaccgccttatcagccggaatcaaccgccacttacatctcctccgtctcctcctccttcccttcaataaccttccaccatcttcccgccgtcacaccgtactcctcctcctccacctctcgccaccaccacgaatctctcctcctagagatcctctgttttagcaacccaagtgtccaccgaactcttttctcactctctcggaatttcaatgtccgagcaatgatcatcgatttcttctgcaccgccgttttagacatcaccgctgacttcacgttcccggtttacttcttctacacctctggagccgcatgtctcgccttttccttctatctcccgaccatcgacgaaacaacccccggaaaaaacctcaaagacattcctacagttcatatccccggcgttcctccgatgaagggctccgatatgcctaaggcggtgctcgaacgagacgatgaggtctacgatgtttttataatgttcggtaaacagctctcgaagtcgtcagggattattatcaatacgtttgatgctttagaaaacagagccatcaaggccataacagaggagctctgttttcgcaatatttatccaattggaccgctcattgtaaacggaagaatcgaagatagaaacgacaacaaggcagtttcttgtctcaattggctggattcgcagccggaaaagagtgttgtgtttctctgttttggaagcttaggtttgttctcaaaagaacaggtgatagagattgctgttggtttagagaaaagtgggcagagattcttgtgggtggtccgtaatccacccgagttagaaaagacagaactggatttgaaatcactcttaccagaaggattcttaagccgaaccgaagacaaagggatggtcgtgaaatcatgggctccgcaagttccggttctgaatcataaagcagtcgggggattcgtcactcattgcggttggaattcaattcttgaagctgtttgtgctggtgtgccgatggtggcttggccgttgtacgctgagcagaggtttaatagagtgatgattgtggatgagatcaagattgcgatttcgatgaatgaatcagagacgggtttcgtgagctctacagaggtggagaaacgagtccaagagataattggggagtgtccggttagggagcgaaccatggctatgaagaacgcagccgaattagccttgacagaaactggttcgtctcataccgcattaactactttactccagtcgtggagcccaaagtga

SEQ ID NO: 6 - protein, 462 aa, Arabidopsis thaliana
MGEEAIVLYPAPPIGHLVSMVELGKTILSKNPSLSIHIILVPPPYQPESTATYISSVSSSFPSITFHHLPAVTPYSSSSTSRHHHESLLLEILCFSNPSVHRTLFSLSRNFNVRAMIIDFFCTAVLDITADFTFPVYFFYTSGAACLAFSFYLPTIDETTPGKNLKDIPTVHIPGVPPMKGSDMPKAVLERDDEVYDVFIMFGKQLSKSSGIIINTFDALENRAIKAITEELCFRNIYPIGPLIVNGRIEDRNDNKAVSCLNWLDSQPEKSVVFLCFGSLGLFSKEQVIEIAVGLEKSGQRFLWVVRNPPELEKTELDLKSLLPEGFLSRTEDKGMVVKSWAPQVPVLNHKAVGGFVTHCGWNSILEAVCAGVPMVAWPLYAEQRFNRVMIVDEIKIAISMNESETGFVSSTEVEKRVQEIIGECPVRERTMAMKNAAELALTETGSSHTALTTLLQSWSPK

SEQ ID NO: 7 - DNA, 1404 bp, Arabidopsis thaliana
  source 1..1404: mol_type = genomic DNA
atgcggaatgtagagctcatcttcatccccacaccaaccgttggtcatcttgttccgtttcttgaatttgctaggcgtctcattgagcaagatgataggatccgtatcacaatcctcttgatgaaactacaaggtcagtctcatctagacacttatgttaaatcaattgcctcctctcaaccgtttgttagattcattgatgtccctgagttagaggagaaacctacacttggtagtacacaatctgtggaagcttatgtgtatgatgttattgagagaaatatccctcttgtgaggaatatagtcatggatattttaacttctcttgcattggatggagttaaggtcaagggattagttgttgactttttctgtctccctatgattgacgttgctaaagatataagtctccctttctatgtgttcttgactacaaattccgggttcttagctatgatgcagtatctagcagatcgacatagtagagatacatcggtttttgtaagaaactcggaagaaatgttgtcgatacctggatttgtaaaccctgtcccagccaatgttctgccgtcagctctgtttgttgaagatggttatgatgcttacgttaagctggccatattgtttacaaaggccaatggaatcctagtgaatagctcctttgatattgagccttactctgtgaatcattttcttcaagaacagaattatccttctgtttatgctgttggccccatatttgacttgaaagcccagcctcatccagagcaggacctaacccgtcgtgacgagttgatgaaatggcttgatgatcaacccgaggcatcggttgtattcctttgttttgggagtatggcaaggttaagaggttctctagtgaaggaaatagctcatggacttgagctatgtcaatatagattcctctggtcactccgtaaagaagaggtgacaaaggatgatttgccagaggggttccttgaccgtgtcgatggacgtggaatgatatgtggttggtctcctcaggtagaaatactggcccataaggcagtgggaggctttgtttctcactgtggatggaactcaatagtagagagtttgtggtttggcgtgccaattgtgacatggccaatgtatgcagagcaacaactcaatgcgtttctgatggtgaaggaactgaagctagctgtggagctgaagcttgattacagggtacatagtgatgagatagtaaacgcaaacgagatagagaccgctattcgttatgtaatggacacggataataatgttgtgaggaaacgagtgatggatatctcgcagatgatccagagagctacgaagaatggtggatcttcgtttgccgcaattgagaaattcatatatgacgtgataggaattaagccctag

SEQ ID NO: 8 - protein, 467 aa, Arabidopsis thaliana
MRNVELIFIPTPTVGHLVPFLEFARRLIEQDDRIRITILLMKLQGQSHLDTYVKSIASSQPFVRFIDVPELEEKPTLGSTQSVEAYVYDVIERNIPLVRNIVMDILTSLALDGVKVKGLVVDFFCLPMIDVAKDISLPFYVFLTTNSGFLAMMQYLADRHSRDTSVFVRNSEEMLSIPGFVNPVPANVLPSALFVEDGYDAYVKLAILFTKANGILVNSSFDIEPYSVNHFLQEQNYPSVYAVGPIFDLKAQPHPEQDLTRRDELMKWLDDQPEASVVFLCFGSMARLRGSLVKEIAHGLELCQYRFLWSLRKEEVTKDDLPEGFLDRVDGRGMICGWSPQVEILAHKAVGGFVSHCGWNSIVESLWFGVPIVTWPMYAEQQLNAFLMVKELKLAVELKLDYRVHSDEIVNANEIETAIRYVMDTDNNVVRKRVMDISQMIQRATKNGGSSFAAIEKFIYDVIGIKP

SEQ ID NO: 9 - DNA, 1455 bp, Arabidopsis thaliana
  source 1..1455: mol_type = genomic DNA
atgaacagagagcaaattcatattttgttcttccccttcatggctcatggccacatgattccactcttagacatggccaagcttttcgctagaagaggagccaaatcaactctcctcacaaccccaataaatgctaagatcttggagaaacccattgaagcattcaaagttcaaaatcctgatctcgaaatcggaatcaagatcctcaatttcccttgtgtagagcttggattgccagaaggatgcgagaaccgtgacttcattaactcataccaaaaatctgactcatttgacttgttcttgaagtttcttttctctaccaagtatatgaaacagcagttggagagtttcattgaaacaaccaaaccgagtgctcttgtagccgatatgttcttcccttgggcaacagaatccgcggagaagatcggtgttccaagacttgtgttccacggcacatcatcctttgccttgtgttgttcgtataacatgaggattcataagccacacaagaaagtcgcttcgagttctactccatttgtaatccctggtctccctggagacatagttattacagaagaccaagccaatgtcaccaacgaagaaactccattcggaaagttttggaaagaagtcagggaatcagagaccagtagctttggtgttttggtgaatagcttctacgagctggaatcatcttatgctgatttttaccgtagttttgtggcgaaaaaagcgtggcatataggtccactttcactatccaacagagggattgcagagaaagccggaagagggaaaaaggcaaacattgatgagcaagaatgcctcaaatggcttgactctaagacacctggctcagtagtttacttgtcctttggtagcggaaccggcttacccaacgaacagctgttagagattgctttcggccttgaaggctctggacaaaatttcatttgggtggttagcaaaaatgaaaaccaagttgggacaggtgaaaatgaagattggttgcctaaagggtttgaagagaggaataaaggaaaagggctgataatacgcggatgggccccgcaagtgctgatacttgaccacaaagcaatcggaggatttgtgacgcattgcggatggaactcgactttggagggcattgccgcagggctgcctatggtgacttggccgatgggggcagaacagttctacaacgagaagttattgacaaaagtgttgagaataggagtgaacgttggagctaccgagttggtgaaaaaaggaaagttgattagtagagcacaagtggagaaggcagtaagggaagtgattggtggtgagaaggcagaggaaaggcggctaagggctaaggagctgggcgagatggctaaagccgctgtggaagaaggagggtcttcttataatgatgtgaacaagtttatggaagagctgaatggtagaaagtag

SEQ ID NO: 10 - protein, 484 aa, Arabidopsis thaliana
MNREQIHILFFPFMAHGHMIPLLDMAKLFARRGAKSTLLTTPINAKILEKPIEAFKVQNPDLEIGIKILNFPCVELGLPEGCENRDFINSYQKSDSFDLFLKFLFSTKYMKQQLESFIETTKPSALVADMFFPWATESAEKIGVPRLVFHGTSSFALCCSYNMRIHKPHKKVASSSTPFVIPGLPGDIVITEDQANVTNEETPFGKFWKEVRESETSSFGVLVNSFYELESSYADFYRSFVAKKAWHIGPLSLSNRGIAEKAGRGKKANIDEQECLKWLDSKTPGSVVYLSFGSGTGLPNEQLLEIAFGLEGSGQNFIWVVSKNENQVGTGENEDWLPKGFEERNKGKGLIIRGWAPQVLILDHKAIGGFVTHCGWNSTLEGIAAGLPMVTWPMGAEQFYNEKLLTKVLRIGVNVGATELVKKGKLISRAQVEKAVREVIGGEKAEERRLRAKELGEMAKAAVEEGGSSYNDVNKFMEELNGRK

SEQ ID NO: 11 - DNA, 1356 bp, Arabidopsis thaliana
  source 1..1356: mol_type = genomic DNA
atggagaagagtaatggcctgcgagtgattctgtttccacttccattacaaggctgcatcaaccctatgattcagctcgccaagatcctccactcaagaggtttttcaatcactgtgatccacacttgcttcaacgcgccaaaagcttcaagccatccactcttcaccttcatacagatccaagatggcttgtctgaaacagagacaagaactcgcgacgtcaaacttctcataacacttctcaaccaaaattgcgagtctccggttcgtgaatgtttgcgtaaactgttgcaatctgccaaggaagagaaacagaggattagctgtttgatcaatgattctggttggatcttcactcaacacttagccaagagtttgaatctcatgagattggcctttaatacctataagatctccttctttcgaagccattttgttcttcctcagctccggcgtgaaatgtttcttccattacaagattcagaacaagatgatccagttgagaagtttccaccgcttagaaagaaagatcttttacggattcttgaagcagattcggtgcagggagactcgtactcggatatgattttggaaaagacaaaggcgtcttcaggtcttatattcatgtcctgtgaagagttggaccaagactcactgagtcaatcacgtgaagattttaaggttccgatatttgcgataggaccttctcatagccattttcctgcttcttctagtagcttgttcacaccggacgagacttgcatcccatggttagacagacaagaagacaaatccgtaatatacgtgagtattgggagcctcgtgaccatcaacgaaacagagctaatggagattgcttggggtctaagtaacagcgaccaaccatttttatgggtcgtccgggttggttcagtcaatggcacggaatggattgaagcaatcccggaatatttcatcaaaaggcttaatgagaagggaaagatagtgaaatgggctccacaacaagaggttctaaagcatcgagctattggaggtttcttgacacataatggttggaactcgacggttgagagtgtttgtgaaggcgtccctatgatctgtttgccttttcgttgggaccaattgttaaatgcaagatttgttagtgatgtatggatggttgggatacatctcgagggtcggattgagagggatgagatcgagagagcgataaggagattattgttggaaactgaaggagaagccatccgagagaggatacaacttcttaaggaaaaagtaggaagatcagttaaacaaaacggttcggcatatcaatctctacaaaatttgattaattatatatcatctttctag

SEQ ID NO: 12 - protein, 451 aa, Arabidopsis thaliana
MEKSNGLRVILFPLPLQGCINPMIQLAKILHSRGFSITVIHTCFNAPKASSHPLFTFIQIQDGLSETETRTRDVKLLITLLNQNCESPVRECLRKLLQSAKEEKQRISCLINDSGWIFTQHLAKSLNLMRLAFNTYKISFFRSHFVLPQLRREMFLPLQDSEQDDPVEKFPPLRKKDLLRILEADSVQGDSYSDMILEKTKASSGLIFMSCEELDQDSLSQSREDFKVPIFAIGPSHSHFPASSSSLFTPDETCIPWLDRQEDKSVIYVSIGSLVTINETELMEIAWGLSNSDQPFLWVVRVGSVNGTEWIEAIPEYFIKRLNEKGKIVKWAPQQEVLKHRAIGGFLTHNGWNSTVESVCEGVPMICLPFRWDQLLNARFVSDVWMVGIHLEGRIERDEIERAIRRLLLETEGEAIRERIQLLKEKVGRSVKQNGSAYQSLQNLINYISSF

SEQ ID NO: 13 - DNA, 1377 bp, Arabidopsis thaliana
  source 1..1377: mol_type = genomic DNA
atgcaggttttgggaatggaggaaaagcctgcaaggagaagcgtagtgttggttccatttccagcacaaggacatatatctccaatgatgcaacttgccaaaacccttcacttaaagggtttctcgatcacagttgttcagactaagttcaattactttagcccttcagatgacttcactcatgattttcagttcgtcaccattccagaaagcttaccagagtctgatttcaagaatctcggaccaatacagtttctgtttaagctcaacaaagagtgtaaggtgagcttcaaggactgtttgggtcagttggtgctgcaacaaagtaatgagatctcatgtgtcatctacgatgagttcatgtactttgctgaagctgcagccaaagagtgtaagcttccaaacatcattttcagcacaacaagtgccacggctttcgcttgccgctctgtatttgacaaactatatgcaaacaatgtccaagctcccttgaaagaaactaaaggacaacaagaagagctagttccggagttttatcccttgagatataaagactttccagtttcacggtttgcatcattagagagcataatggaggtgtataggaatacagttgacaaacggacagcttcctcggtgataatcaacactgcgagctgtctagagagctcatctctgtcttttctgcaacaacaacagctacaaattccagtgtatcctataggccctcttcacatggtggcctcagctcctacaagtctgcttgaagagaacaagagctgcatcgaatggttgaacaaacaaaaggtaaactcggtgatatacataagcatgggaagcatagctttaatggaaatcaacgagataatggaagtcgcgtcaggattggctgctagcaaccaacacttcttatgggtgatccgaccagggtcaatacctggttccgagtggatagagtccatgcctgaagagtttagtaagatggttttggaccgaggttacattgtgaaatgggctccacagaaggaagtactttctcatcctgcagtaggagggttttggagccattgtggatggaactcgacactagaaagcatcggccaaggagttccaatgatctgcaggccattttcgggtgatcaaaaggtgaacgctagatacttggagtgtgtatggaaaattgggattcaagtggagggtgagctagacagaggagtggtcgagagagctgtgaagaggttaatggttgacgaagaaggagaggagatgaggaagagagctttcagtttaaaagagcaacttagagcctctgttaaaagtggaggctcttcacacaactcgctagaagagtttgtacacttcataaggactctatga

SEQ ID NO: 14 - protein, 458 aa, Arabidopsis thaliana
MQVLGMEEKPARRSVVLVPFPAQGHISPMMQLAKTLHLKGFSITVVQTKFNYFSPSDDFTHDFQFVTIPESLPESDFKNLGPIQFLFKLNKECKVSFKDCLGQLVLQQSNEISCVIYDEFMYFAEAAAKECKLPNIIFSTTSATAFACRSVFDKLYANNVQAPLKETKGQQEELVPEFYPLRYKDFPVSRFASLESIMEVYRNTVDKRTASSVIINTASCLESSSLSFLQQQQLQIPVYPIGPLHMVASAPTSLLEENKSCIEWLNKQKVNSVIYISMGSIALMEINEIMEVASGLAASNQHFLWVIRPGSIPGSEWIESMPEEFSKMVLDRGYIVKWAPQKEVLSHPAVGGFWSHCGWNSTLESIGQGVPMICRPFSGDQKVNARYLECVWKIGIQVEGELDRGVVERAVKRLMVDEEGEEMRKRAFSLKEQLRASVKSGGSSHNSLEEFVHFIRTL

SEQ ID NO: 15 - DNA, 1476 bp, Cannabis sativa
  source 1..1476: mol_type = genomic DNA
atggcctctgaaccatacaaattgcatttgatcgttatcccattcatggctccaggtcattttattccaatggctgatatggctagaaagttggctgaacatggtgctatgattactttgattaccttgccagttattgccgccagaattagaccaattattgaacaagctaccgagaactccaacttgaagattcaattggttcaagtctccttgccattgcatgaatttggtttgcctgaaggttgtgataccgttgatttggttccatctagaaacctgttgctgtctttcttcattgccttggatgaattgcaacagccaatcgaacaagttgtctctgaattgaaacctagaccatcctgtattattgccgacaaacatttgccatggactgctgaaattgctaccaagtttggtattccaagagttttgttcgatggtatgtcttgcttctctttgttgtgcaaccatatgatcagaaagtcccaagttcatttgtccgttccaatgtctgttccatttgttgttccaggtatgccagatcatttggagttcactagaaatcaattggctgctgacttgtaccctaatttggaatttggtcaaaagttccacgacaggatcagagaatctgaagaaggtgctgacggttttttggttaactctttcgaagaattggagtggaagtacgttgagggttacagaaaagaaaaagttggtaaggcttggtgcattggtccagtttctttgtttaacaagaccaagttggaagttgcccaaagaggtaacaatccagctggtgctgttgacgaaaaacaatgtactgaatggttggattcttggccaaagtcttgtgttgtttatgcttgtttgggttccgtttccagattattgattccacagatgatcgaattgggtgttgctttggaagcttctaacaaaccattcatctgggttatcagaggttacgatcaagaggaagaaatcgaaaagtggatctccgaatctggttttaaagaaaggactaagtccagagccttgttgatttttggttgggctccacaagttctgattttgtctcattcttctgttggtggtttcttgactcattgtggttggaattctaccttggaaggtattacttatggcaagccaatgattacatggccaatgttcgctgaacaattctacaatcaaaagttgatcgtccaggttttgaaggttggtgaatctgttggtccaaaattcgttgttccatatggtagagaagaggaattcggtgttttcgttttgtccaaggatattttggaagccatcgaaaaggttatggcccaagacaaagaaggtgaagaacgtagagaaagagtcaagagattgtcagatatggctcaaaaggctattgaggaaggtggttcttcttacttggatatgaagttgttcatcgaggacatcagaaacttgcacatctcttga

SEQ ID NO: 16 - protein, 491 aa, Cannabis sativa
MASEPYKLHLIVIPFMAPGHFIPMADMARKLAEHGAMITLITLPVIAARIRPIIEQATENSNLKIQLVQVSLPLHEFGLPEGCDTVDLVPSRNLLLSFFIALDELQQPIEQVVSELKPRPSCIIADKHLPWTAEIATKFGIPRVLFDGMSCFSLLCNHMIRKSQVHLSVPMSVPFVVPGMPDHLEFTRNQLAADLYPNLEFGQKFHDRIRESEEGADGFLVNSFEELEWKYVEGYRKEKVGKAWCIGPVSLFNKTKLEVAQRGNNPAGAVDEKQCTEWLDSWPKSCVVYACLGSVSRLLIPQMIELGVALEASNKPFIWVIRGYDQEEEIEKWISESGFKERTKSRALLIFGWAPQVLILSHSSVGGFLTHCGWNSTLEGITYGKPMITWPMFAEQFYNQKLIVQVLKVGESVGPKFVVPYGREEEFGVFVLSKDILEAIEKVMAQDKEGEERRERVKRLSDMAQKAIEEGGSSYLDMKLFIEDIRNLHIS

SEQ ID NO: 17 - DNA, 1383 bp, Arabidopsis thaliana
  source 1..1383: mol_type = genomic DNA
atggtcgacaagagagaagaagttatgcacgtagccatgtttccatggctagctatgggtcatctccttccttttcttcgtctctccaagttactagctcaaaagggtcacaagatctctttcatatcaacaccaagaaacatcgaaagacttcctaaattacaatcaaacctcgcctcctccatcaccttcgtctctttccctctccctcccatctcaggcttgcctccttcttcagaatcatccatggacgttccttacaacaagcaacagtctcttaaagccgcttttgatcttcttcagccaccgttgaaagagtttctccgacggtcttctccggattggatcatatacgactatgcttctcactggcttccttctattgcggccgagcttggaatctctaaggctttctttagtctctttaacgcagctactctctgtttcatgggaccgtcttcgtctttgattgaagaaattagatcaacgccggaagatttcacggtggtgccaccgtgggtcccgttcaagtcaaacatcgtgtttcgttatcatgaagttactagatacgttgagaagacagaggaagatgtaaccggagtctctgactcagttcggtttggttactcgattgacgaaagcgatgcggtttttgtccgtagctgtccggagtttgaaccggaatggtttggtttactaaaagacctgtaccgtaaaccggtatttccaatcgggtttttgcctccggttattgaagacgacgatgccgttgatactacatgggttcgtataaagaagtggctcgacaagcaacggcttaattcagttgtttacgtgtcacttggcaccgaagcgagtcttcgtcatgaggaagtaactgagctagctcttgggttagagaagtcagagacaccgttcttttgggtcctaaggaacgagccaaagattccagatgggttcaaaacacgagtcaagggacgtggaatggttcatgttggttgggttccacaagtgaaaatacttagtcacgagtcagtaggagggttcttgacacattgtggttggaactcagtggtggaagggttagggtttggtaaagttccaatcttttttccggtgttgaatgagcaaggacttaatacgaggttgttgcatgggaaaggacttggtgttgaggtttcaagagatgagagagatgggtcgtttgattctgactcggtcgctgactcgattaggttggtgatgattgatgatgctggcgaggagataagggctaaggctaaagtgatgaaggatttgtttgggaacatggatgagaatattcgttatgttgacgaacttgttaggtttatgagaagtaaaggatcatcatcatcatcatga

SEQ ID NO: 18 - protein, 460 aa, Arabidopsis thaliana
MVDKREEVMHVAMFPWLAMGHLLPFLRLSKLLAQKGHKISFISTPRNIERLPKLQSNLASSITFVSFPLPPISGLPPSSESSMDVPYNKQQSLKAAFDLLQPPLKEFLRRSSPDWIIYDYASHWLPSIAAELGISKAFFSLFNAATLCFMGPSSSLIEEIRSTPEDFTVVPPWVPFKSNIVFRYHEVTRYVEKTEEDVTGVSDSVRFGYSIDESDAVFVRSCPEFEPEWFGLLKDLYRKPVFPIGFLPPVIEDDDAVDTTWVRIKKWLDKQRLNSVVYVSLGTEASLRHEEVTELALGLEKSETPFFWVLRNEPKIPDGFKTRVKGRGMVHVGWVPQVKILSHESVGGFLTHCGWNSVVEGLGFGKVPIFFPVLNEQGLNTRLLHGKGLGVEVSRDERDGSFDSDSVADSIRLVMIDDAGEEIRAKAKVMKDLFGNMDENIRYVDELVRFMRSKGSSSSS

SEQ ID NO: 19 - DNA, 1377 bp, Stevia rebaudiana
  source 1..1377: mol_type = genomic DNA
atggaaaataaaacggagaccaccgttcgccggcgccggagaataatattattcccggtaccatttcaaggccacattaacccaattcttcagctagccaatgtgttgtactctaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcacggatgctctttggtacttcgcgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaagagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttcaatggttagaccaacaaccgccaagttcggtactgtatgttagttttggtagtactagtgaagtggatgagaaagatttcttggaaatagctcgtgggttggttgatagcaagcagtcgtttttatgggtggttcgacctgggtttgtcaagggttcgacgtgggtcgaaccgttgccagatgggttcttgggtgaaagaggacgtattgtgaaatgggttccacagcaagaagtgctagctcatggagcaataggcgcattctggactcatagcggatggaactctacgttggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaataagaagagttatggtggatgaagaaggagaatacattagacagaatgcaagagttttgaaacaaaaggcagatgtttctttgatgaagggtggttcgtcttacgaatcattagagtctctagtttcttacatttcatcgttgtaa

SEQ ID NO: 20 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTRASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 21 - DNA, 1467 bp, Arabidopsis thaliana
  source 1..1467: mol_type = genomic DNA
atgggatcccgttttgtttctaacgaacaaaaaccacacgtagtttgcgtgccttacccagctcaaggccacattaaccctatgatgaaagtggctaaactcctccacgtcaaaggcttccacgtcaccttcgtcaacaccgtctacaaccacaaccgtctactccgatcccgtggggccaacgcactcgatggacttccttccttccagttcgagtcaatacctgacggtcttccggagactggcgtggacgccacgcaggacatccctgccctttccgagtccacaacgaaaaactgtctcgttccgttcaagaagcttctccagcggattgtcacgagagaggatgtccctccggtgagctgtattgtatcagatggttcgatgagctttactcttgacgtagcggaagagcttggtgttccggagattcatttttggaccactagtgcttgtggcttcatggcttatctacacttttatctcttcatcgagaagggtttatgtccagtaaaagatgcgagttgcttgacgaaggaatacttggacacagttatagattggataccgtcaatgaacaatgtaaaactaaaagacattcctagttttatacgtaccactaatcctaacgacataatgctcaacttcgttgtccgtgaggcatgtcgaaccaaacgtgcctctgctatcattctgaacacgtttgatgaccttgaacatgacataatccagtctatgcaatccattttaccaccggtttatccaatcggaccgcttcatctcttagtaaacagggagattgaagaagatagtgagattggaaggatgggatcaaatctatggaaagaggagactgagtgcttgggatggcttaatactaagtctcgaaatagcgttgtttatgttaactttgggagcataacaataatgaccacggcacagcttttggagtttgcttggggtttggcggcaacgggaaaggagtttctatgggtgatgcggccggattcagtagccggagaggaggcagtgattccaaaagagtttttagcggagacagctgatcgaagaatgctgacaagttggtgtcctcaggagaaagttctttctcatccggcggtcggagggttcttgacccattgcgggtggaattcgacgttagaaagtctttcatgcggagttccaatggtatgttggccattttttgctgagcaacaaacaaattgtaagttttcttgtgatgaatgggaggttggtattgagatcggtggagatgtcaagaggggagaggttgaggcggtggttagagagctcatggatggagagaaaggaaagaaaatgagagagaaggctgtagagtggcggcgcttggccgagaaagctacaaagcttccgtgtggttcgtcggtgataaattttgagacgattgtcaacaaggttctcttgggaaagatccctaacacgtaa

SEQ ID NO: 22 - protein, 488 aa, Arabidopsis thaliana
MGSRFVSNEQKPHVVCVPYPAQGHINPMMKVAKLLHVKGFHVTFVNTVYNHNRLLRSRGANALDGLPSFQFESIPDGLPETGVDATQDIPALSESTTKNCLVPFKKLLQRIVTREDVPPVSCIVSDGSMSFTLDVAEELGVPEIHFWTTSACGFMAYLHFYLFIEKGLCPVKDASCLTKEYLDTVIDWIPSMNNVKLKDIPSFIRTTNPNDIMLNFVVREACRTKRASAIILNTFDDLEHDIIQSMQSILPPVYPIGPLHLLVNREIEEDSEIGRMGSNLWKEETECLGWLNTKSRNSVVYVNFGSITIMTTAQLLEFAWGLAATGKEFLWVMRPDSVAGEEAVIPKEFLAETADRRMLTSWCPQEKVLSHPAVGGFLTHCGWNSTLESLSCGVPMVCWPFFAEQQTNCKFSCDEWEVGIEIGGDVKRGEVEAVVRELMDGEKGKKMREKAVEWRRLAEKATKLPCGSSVINFETIVNKVLLGKIPNT

SEQ ID NO: 23 - DNA, 1407 bp, Arabidopsis thaliana
  source 1..1407: mol_type = genomic DNA
atgggtgtttttggatcgaatgaatcgtcaagcatgagtattgtgatgtatccgtggttagcctttggtcacatgactccttttcttcacctatccaacaagctcgcagagaaaggtcacaagattgttttcttgcttcccaagaaagcactaaaccagcttgaacctcttaatctctacccaaatctcatcactttccacaccatctctatccctcaggtcaaagggctccctccgggtgcggagacaaactccgacgtccctttcttcttgacacatttgcttgcagttgcaatggaccaaacccggccagaggtcgagaccattttccgtacaatcaaaccggacttggttttctatgattctgcccattggataccggaaattgctaaaccgatcggtgctaaaaccgtttgcttcaacatcgttagcgctgcgtcaatcgcactgtctcttgtcccttctgcggagagagaggtcattgatggcaaggaaatgtcaggggaggagttagctaagacgcctctaggttacccatcttcgaaagtagtcttacgtccgcacgaagcaaaatccctgagtttcgtgtggaggaagcacgaggcgattggctctttctttgatgggaaagttaccgcgatgagaaactgcgacgcaatcgctataaggacttgccgtgagacagaaggcaaattctgcgattacataagtaggcagtacagtaaaccggtttacctaacaggaccggttctccctggatcccaacctaatcagccctccttagatcctcaatgggcggagtggctagccaaattcaaccacggttcggttgtgttctgcgctttcggtagccaacccgttgtaaacaagatagatcagtttcaagaactctgtttaggtctagaatcaactggttttccgtttctggttgccattaagcctccttcgggtgtatcaaccgtcgaggaagccttaccggaaggattcaaagagagggttcaaggacgtggcgttgtgtttggaggttggattcagcaaccgttggtgttgaaccatccttcagtgggttgttttgttagccattgcgggtttgggtcgatgtgggagtcgttgatgagtgattgtcagatcgttttggttccgcagcacggagaacagattttgaacgcaaggctgatgacggaggagatggaggtggcggttgaagtggagagggaaaagaaagggtggttctcgcggcaaagcttggagaatgctgtgaagagtgtgatggaggaaggtagtgagatcggtgagaaagtgaggaagaatcatgacaagtggagatgtgttttgactgactctggtttttcagatggttatattgataagtttgaacaaaatttaattgaacttgtgaagtcatga

SEQ ID NO: 24 - protein, 468 aa, Arabidopsis thaliana
MGVFGSNESSSMSIVMYPWLAFGHMTPFLHLSNKLAEKGHKIVFLLPKKALNQLEPLNLYPNLITFHTISIPQVKGLPPGAETNSDVPFFLTHLLAVAMDQTRPEVETIFRTIKPDLVFYDSAHWIPEIAKPIGAKTVCFNIVSAASIALSLVPSAEREVIDGKEMSGEELAKTPLGYPSSKVVLRPHEAKSLSFVWRKHEAIGSFFDGKVTAMRNCDAIAIRTCRETEGKFCDYISRQYSKPVYLTGPVLPGSQPNQPSLDPQWAEWLAKFNHGSVVFCAFGSQPVVNKIDQFQELCLGLESTGFPFLVAIKPPSGVSTVEEALPEGFKERVQGRGVVFGGWIQQPLVLNHPSVGCFVSHCGFGSMWESLMSDCQIVLVPQHGEQILNARLMTEEMEVAVEVEREKKGWFSRQSLENAVKSVMEEGSEIGEKVRKNHDKWRCVLTDSGFSDGYIDKFEQNLIELVKS

SEQ ID NO: 25 - DNA, 1401 bp, Arabidopsis thaliana
  source 1..1401: mol_type = genomic DNA
atggccgagccaaaaccgaagcttcatgttgcagtgttcccatggttagctttaggtcacatgattccttacttgcaactctcaaagctcatagcaaggaaaggccatactgtgtccttcatctccacagctcgtaacatttcacgtcttcccaatatatcctccgacctttccgtgaatttcgtttctttgccgttaagtcaaaccgtcgaccatctcccagagaacgctgaggccaccactgatgtcccggagactcacatagcttatctgaagaaagcatttgatgggctttctgaagctttcacagagtttttagaagcttccaaaccaaactggatagtgtatgatatcttgcaccattgggtcccgcctatcgctgagaagctcggcgtgagacgagccatcttctgcacgttcaacgcagcttccatcatcatcatcggtgggccagcatcagtcatgattcaaggtcatgaccctcgaaagactgctgaagatcttatcgtgcctccaccatgggtcccgtttgagaccaacatagtttaccgtctctttgaagctaagaggatcatggagtatcccacggcaggtgtaactggagttgaattgaacgacaactgtagattgggtttggcttacgttggctctgaggttattgtgattagatcatgtatggaactcgaacctgagtggattcaattgctcagtaaactccaaggaaagcctgtgattccaattggtttactcccggctacaccaatggatgatgcagatgacgagggaacatggttagacatcagagaatggctagacagacatcaagcaaagtctgtggtttatgtagccttaggaactgaagtgacaattagtaacgaagagattcaaggtttagctcatgggttggagctttgcaggttacctttcttttggacgctaaggaagaggactagagcttctatgctactacctgatgggttcaaagagagagtcaaagagcgtggagtcatttggaccgagtgggtacctcagaccaagatactgagccatggttcagttggtgggtttgttactcattgtggttggggatcagctgtggaagggcttagctttggtgtccctttgatcatgtttccatgtaacctagaccagccgctagtggctaggttgctcagtgggatgaatataggcttggagattccaaggaatgagcgagacgggctgttcacgagtgcttctgttgcagagacaatcagacatgttgttgtggaagaagaaggaaagatctacaggaacaatgctgcatctcagcaaaagaaaatattcgggaacaagagattgcaagatcagtatgcggatggttttatcgagtttctggagaatcctatagcaggagtgtag

SEQ ID NO: 26 - protein, 466 aa, Arabidopsis thaliana
MAEPKPKLHVAVFPWLALGHMIPYLQLSKLIARKGHTVSFISTARNISRLPNISSDLSVNFVSLPLSQTVDHLPENAEATTDVPETHIAYLKKAFDGLSEAFTEFLEASKPNWIVYDILHHWVPPIAEKLGVRRAIFCTFNAASIIIIGGPASVMIQGHDPRKTAEDLIVPPPWVPFETNIVYRLFEAKRIMEYPTAGVTGVELNDNCRLGLAYVGSEVIVIRSCMELEPEWIQLLSKLQGKPVIPIGLLPATPMDDADDEGTWLDIREWLDRHQAKSVVYVALGTEVTISNEEIQGLAHGLELCRLPFFWTLRKRTRASMLLPDGFKERVKERGVIWTEWVPQTKILSHGSVGGFVTHCGWGSAVEGLSFGVPLIMFPCNLDQPLVARLLSGMNIGLEIPRNERDGLFTSASVAETIRHVVVEEEGKIYRNNAASQQKKIFGNKRLQDQYADGFIEFLENPIAGV

SEQ ID NO: 27 - DNA, 1344 bp, Arabidopsis thaliana
  source 1..1344: mol_type = genomic DNA
atggagactagagaaacaaaaccagtgatctttctcttccctttccctttacaaggtcacttaaacccaatgtttcagctcgccaacatcttcttcaacagaggcttctccatcactgtgatccacactgagttcaactctccaaactcttccaatttccctcatttcactttcgtatccatccccgatagcttgtctgaacctgaatcctatcccgatgtcatcgagattctccatgacctcaattccaagtgtgttgctccttttggtgattgcttaaagaagcttatatctgaagaaccaacagcagcttgtgtgattgttgacgctctttggtacttcactcacgatttaaccgagaaattcaatttcccgaggattgttctccgaaccgttaacctctcagctttcgtcgctttctcaaagtttcatgttttacgagagaaagggtatctttctttacaagagactaaggcagactcaccggttccggagcttccgtatcttagaatgaaggatcttccatggttccagacagaagatccaagatcaggggataagttacagataggtgtgatgaagtcactaaagtcttcctcaggaatcatattcaacgccattgaagatcttgaaacagatcagcttgatgaagcccgcatagaattcccagttccactcttctgtattggaccctttcacaggtacgtttcagcttcatccagtagcttacttgcacacgacatgacttgtctctcctggttagacaagcaagcaacaaattccgtaatctacgcaagtcttggaagcattgcttcgatcgatgaatctgaattcttggagattgcttggggtctaagaaacagcaaccaaccttttctatgggtggttagacccggtttaatccacgggaaagaatggatcgagattctgcctaaagggttcatcgaaaatctcgagggccggggtaaaatagtgaaatgggcacctcagcctgaagttttagctcaccgtgcaacaggcggattcttaacacattgtggatggaactcaacacttgagggcatatgtgaagctataccaatgatatgcagaccatcttttggggaccagagggtgaatgctagatacattaacgatgtttggaagatcggattgcatttggaaaacaaggtagagagactagtgatcgaaaacgcggttagaacactaatgacgagctcggaaggggaagagatccgcaagaggattatgcccatgaaggaaactgttgaacaatgccttaagcttggaggttcatcatttcggaatctcgaaaacttaattgcttatatattgtctttctaa

SEQ ID NO: 28 - protein, 447 aa, Arabidopsis thaliana
METRETKPVIFLFPFPLQGHLNPMFQLANIFFNRGFSITVIHTEFNSPNSSNFPHFTFVSIPDSLSEPESYPDVIEILHDLNSKCVAPFGDCLKKLISEEPTAACVIVDALWYFTHDLTEKFNFPRIVLRTVNLSAFVAFSKFHVLREKGYLSLQETKADSPVPELPYLRMKDLPWFQTEDPRSGDKLQIGVMKSLKSSSGIIFNAIEDLETDQLDEARIEFPVPLFCIGPFHRYVSASSSSLLAHDMTCLSWLDKQATNSVIYASLGSIASIDESEFLEIAWGLRNSNQPFLWVVRPGLIHGKEWIEILPKGFIENLEGRGKIVKWAPQPEVLAHRATGGFLTHCGWNSTLEGICEAIPMICRPSFGDQRVNARYINDVWKIGLHLENKVERLVIENAVRTLMTSSEGEEIRKRIMPMKETVEQCLKLGGSSFRNLENLIAYILSF

SEQ ID NO: 29 - DNA, 1359 bp, Arabidopsis thaliana
  source 1..1359: mol_type = genomic DNA
atggcagagattcgccagagaagagtgttgatggtcccagcaccgttccaaggccatttaccttcgatgatgaatctagcgtcctacctttcttcccaaggcttttcaatcacaatcgttagaaacgaattcaatttcaaagatatctcccataatttccctggtataaaattcttcaccatcaaggacggcttgtcagaatctgacgtgaagtctctgggtctccttgaatttgtcctggagcttaactctgtctgtgaacccctattgaaagagtttctaaccaaccatgatgatgttgttgactttatcatttatgatgaatttgtttacttccctcgacgtgttgcggaagatatgaatctgccaaagatggtctttagcccttcttccgccgctacctcgatcagccggtgtgtgcttatggagaaccaatcaaatgggttacttcctccacaagacgcaagatctcaactagaagaaacggtgccagagtttcatccctttcgtttcaaagatctgccttttacagcttatggatctatggagagattaatgatactttacgagaatgtaagcaatagagcctcatcttctggcataatacacaactcttcggattgcttagagaactcattcataacaactgcacaagagaaatggggagttccggtatacccggttggtccactccatatgaccaattccgcaatgtcatgtccaagtttatttgaagaagaaagaaactgtcttgaatggcttgagaagcaagaaacaagctcagtgatctacataagcatggggagcttggcgatgacacaagatatagaggctgtggagatggccatgggatttgtccagagtaatcaacccttcttgtgggtgatccgaccaggctctataaacggacaagaatctttagacttcttaccggaacagttcaaccaaacggtgaccgatggaagaggttttgttgtgaaatgggccccacaaaaagaggtattaaggcatagagcagtgggagggttttggaaccatggtggatggaactcgtgcttggagagcataagcagtggtgtaccaatgatttgtaggccgtattctggtgatcagagggtgaatactcgacttatgtcacatgtttggcaaaccgcgtatgagatcgaaggtgaattggaaagaggagctgttgagatggccgtgaggaggctcattgtggatcaagaaggtcaggagatgagaatgagagccaccatattgaaggaagaggttgaagcctctgtcacaaccgaaggctcttctcacaattctttaaacaatttggtccatgcaataatgatgcaaattgacgaacaatga

SEQ ID NO: 30 - protein, 452 aa, Arabidopsis thaliana
MAEIRQRRVLMVPAPFQGHLPSMMNLASYLSSQGFSITIVRNEFNFKDISHNFPGIKFFTIKDGLSESDVKSLGLLEFVLELNSVCEPLLKEFLTNHDDVVDFIIYDEFVYFPRRVAEDMNLPKMVFSPSSAATSISRCVLMENQSNGLLPPQDARSQLEETVPEFHPFRFKDLPFTAYGSMERLMILYENVSNRASSSGIIHNSSDCLENSFITTAQEKWGVPVYPVGPLHMTNSAMSCPSLFEEERNCLEWLEKQETSSVIYISMGSLAMTQDIEAVEMAMGFVQSNQPFLWVIRPGSINGQESLDFLPEQFNQTVTDGRGFVVKWAPQKEVLRHRAVGGFWNHGGWNSCLESISSGVPMICRPYSGDQRVNTRLMSHVWQTAYEIEGELERGAVEMAVRRLIVDQEGQEMRMRATILKEEVEASVTTEGSSHNSLNNLVHAIMMQIDEQ

SEQ ID NO: 31 - DNA, 1380 bp, Cannabis sativa
  source 1..1380: mol_type = genomic DNA
atggttcagccaagattcttgattttggcttttccattgcagggtactattaacccatgtttgaacttggctaaccagttgattagagttgctaacgctcaagttactttcgttacttctgttaacgcccacagattgattatgactactcatactgttgctaccacctccaacaatttgttgtctttttctccattcttcgacggttacgatgaaggtgttactgatggtaaaggtttccatgatcacttcgtcgaattcaaaagaagaggttggcaagctgttggtgatattttggaattgggtttcaaagaaggtaggccatacacttgtttggtctactctattttgttgacttgggctgctgatgttgctgctacacataatgttccagcttctatgttttggatgcaaccagctactgttttcgatgtgtattactactacttccacggccacaaagaaattatctgtgctaacactaagaaccacagcttctcattgtctttcccaagaattccattgaccatgaacttgaaggatctgccatctttgatggttgactctaactactcttacatcttgaccatgttgcacgaaatgtacaaggacttcgaaaaagagtctaacaacaccaagatcatcctggttaacactttcgatgaattggaaccagatgctttgagagccattaacaagttcaacttgattggtatcggtcccttgattacttctaagacctcattctctttcagaaactacatcgaatggttgaacacgaagccaaaaaagaccgttgtttacgtttccttcggttccattctgatcttgaaaaaacaacagatggacgaaattgccaagggtttgttggaatttggtcatccatttctgtgggtcatcaaagagaagaactcctcatctaaagaaggtgtccacgaagataacatcaagaacgagttgtcctacaaagaggaattggaaaagttgggtatgatcgttccatggtgttctcaaatggaagtgttgagaaatgaatccttgggttgtttcgttacacattgtggttggaactctaccttggaatctatagtttctggtgttccagttgttgcttttccacaatggactgatcaacaaacaaacgccaagttgattgaagagatgtggaagattggtgtcagagttaagccagatgaagatggtattgtcaagtccgaagaaatcaagagatgtttggagttggtcatgtccaagaacgaaaacagaactgaaatcgtgaagaacgtcaagaagtggcaaaacttgacaaaagaagctatgagagaaggcggttcttctgaaaaaaacttgatcaccttcgtgaagtccatccaccaatga

SEQ ID NO: 32 - protein, 459 aa, Cannabis sativa
MVQPRFLILAFPLQGTINPCLNLANQLIRVANAQVTFVTSVNAHRLIMTTHTVATTSNNLLSFSPFFDGYDEGVTDGKGFHDHFVEFKRRGWQAVGDILELGFKEGRPYTCLVYSILLTWAADVAATHNVPASMFWMQPATVFDVYYYYFHGHKEIICANTKNHSFSLSFPRIPLTMNLKDLPSLMVDSNYSYILTMLHEMYKDFEKESNNTKIILVNTFDELEPDALRAINKFNLIGIGPLITSKTSFSFRNYIEWLNTKPKKTVVYVSFGSILILKKQQMDEIAKGLLEFGHPFLWVIKEKNSSSKEGVHEDNIKNELSYKEELEKLGMIVPWCSQMEVLRNESLGCFVTHCGWNSTLESIVSGVPVVAFPQWTDQQTNAKLIEEMWKIGVRVKPDEDGIVKSEEIKRCLELVMSKNENRTEIVKNVKKWQNLTKEAMREGGSSEKNLITFVKSIHQ

SEQ ID NO: 33 - DNA, 1347 bp, Cannabis sativa
  source 1..1347: mol_type = genomic DNA
atgtccaagggtcataccattccattattgcatttggccagagtcttgttgaacagacatgttactgttaccattttcactaccccagctaacagatctttcatcactaagtttttgccagctacttctgctgctatagtcgaattgccatttccaaagaatattccaggtgttccaaacggtgttgaaaacactgaagatttcccaaccatgtccatgtctatgttctactcattggttttgggtacgcagaatatgaagccagatttggatagagccttggaaaacattcaaaccccagtttctttcatggtttccgatggttttttgtggtggactttggattctgcttctaagttgggttttcccagattggttttttacggtatgtctcattatgccatggccgtttaccattctttgttcaattctaagaagccaaagcagactgaaaccgaaactgttgtttctgatttcccatggattaagttgaccagatctgaatatgatccatccgctcaaaatggtgaagatcaatctttggctcacgagtttatgtctaaagctactgaagctaccaacaactctttcggtatgatctacaactccttctacgaattggaacctatgttcaccgattactggaatcaaaaagttggtccaaaatcttggccattgggtccattgtgtttacacgatatgaagatcgaatccagaggtttggttgttcatccatggttggacgaaaaagaatcttcttctgtcttgtacgttgcctttggttctcaagctactgtttcttctgaacaggttagagaaattgccaaaggtttggaatactccaacgttaactttttctgggtcttgagaaagttggaaccagaagaaaacaagttcttggaagaattcgaaaagagggtcaagaacagaggtatcgttgttagagattgggttaaccagatggaaatcttgaaacacaagtccgttaagggtttcttctctcattgtggttggaactctgttatggaatctttgtctgctggtttgccaattttgggtttcccaatgatggctgaacaacatattaacgccaagatggttgtcgaagaaatcaagataggtctgagagttaagtcctacgatggttctttgaatggtatcgttaagtccgaagaggttagcaagatggtcaaagaattgatggaaggtgaagtcggtaaagagatgagaaagaaggttgaagaatttgctgttatggctcataaggccgttcaaaaaggtggttcttcttgggaaactttggacttgttgttgtcctctaccaagcaaagaatccatcactactga

SEQ ID NO: 34 - protein, 448 aa, Cannabis sativa
MSKGHTIPLLHLARVLLNRHVTVTIFTTPANRSFITKFLPATSAAIVELPFPKNIPGVPNGVENTEDFPTMSMSMFYSLVLGTQNMKPDLDRALENIQTPVSFMVSDGFLWWTLDSASKLGFPRLVFYGMSHYAMAVYHSLFNSKKPKQTETETVVSDFPWIKLTRSEYDPSAQNGEDQSLAHEFMSKATEATNNSFGMIYNSFYELEPMFTDYWNQKVGPKSWPLGPLCLHDMKIESRGLVVHPWLDEKESSSVLYVAFGSQATVSSEQVREIAKGLEYSNVNFFWVLRKLEPEENKFLEEFEKRVKNRGIVVRDWVNQMEILKHKSVKGFFSHCGWNSVMESLSAGLPILGFPMMAEQHINAKMVVEEIKIGLRVKSYDGSLNGIVKSEEVSKMVKELMEGEVGKEMRKKVEEFAVMAHKAVQKGGSSWETLDLLLSSTKQRIHHY

SEQ ID NO: 35 - DNA, 1539 bp, Cannabis sativa
  source 1..1539: mol_type = genomic DNA
atgtccaaagaaatctgggttgttccattctttggtcaaggtcatttgttcccatctatggaattgtgcaagcaaattgcctccagaaacattaacaccttgttggttattccctccaacctgtctttttctatcccatcttctttgagacaataccccttgttgcaaatcgttgaaattcaacctacttctgctccatctgctcaaccaggtccagatccaattgatcaaccaccacatggtaatccagatcaattcgaaatgggtctagaaaacttgttgcaggctcaagttactggtccagattctgttagaccaatttgcgctattttggatgttatgatggattggactaccgaggttttcaacaagttcgatattccaactatcggcttctttacttctggtgcttgttctgctgctatggaatatggtttgtggaaagctcaacctatcgatttgaaaccaggtgaagttagattattgccaggtttgccagaagaaatggctgttacttacttggacactaatcaaagaccacatcaacctccaggtccaccattgcatttgtttggtgttggtcatcaaggtccttttgttgatggtgctcatagaccaccaggtcctccaccaccaccatatttgggtagagctggtccaagacaaaggggtccaccaaaacatggttcacaaccaccatgggttgatggtttgaaaggttctattgccttgatgattaacacgtgcgattacttggaaaggccattcattgaatacttgaccaagcaaatcggtaaaccagtttggggtgtaggtccattattaccagaacaattttggaactccgtgtcctctaattccatattgcatgatcacgaaatcaggaccaacagacaatctaacgtttccgaagatgatgtcatccaatggttggattctaaacctagaggttctgtcttgtacgtttgtttcggtactgaagtttctccatccatggaagaatactctgaattggctgatgctttggaagcttctactcaaccttttatttgggtcgttcattctggtactggtagaagtggtccaccaccttctagaggtccaactcaagaggattattttccacatggtttggcttctcaagttggtccaaaaggtttgattattaacggttgggctccacagttgttgatcttgtctcattcttctattggtggtttcttgactcattgtggttggaactctactgttgaagctattggtttaggtgttccattattggcttggccaattagaggtgatcaaaactacaatgccaagttggttgttgcccatttgaagttgggtttcatgatctctgataacctgtccgagaagattaagaagcacgaaattgtcaagggtatcaagactttgatgggtgatgatgatattaagtccagagctagaaacttggctgccatttttcaaaagggtttcccaatttcttctaccactaacttggatgtgttcagggatatgatcaacaatttcacgtaa

SEQ ID NO: 36 - protein, 512 aa, Cannabis sativa
MSKEIWVVPFFGQGHLFPSMELCKQIASRNINTLLVIPSNLSFSIPSSLRQYPLLQIVEIQPTSAPSAQPGPDPIDQPPHGNPDQFEMGLENLLQAQVTGPDSVRPICAILDVMMDWTTEVFNKFDIPTIGFFTSGACSAAMEYGLWKAQPIDLKPGEVRLLPGLPEEMAVTYLDTNQRPHQPPGPPLHLFGVGHQGPFVDGAHRPPGPPPPPYLGRAGPRQRGPPKHGSQPPWVDGLKGSIALMINTCDYLERPFIEYLTKQIGKPVWGVGPLLPEQFWNSVSSNSILHDHEIRTNRQSNVSEDDVIQWLDSKPRGSVLYVCFGTEVSPSMEEYSELADALEASTQPFIWVVHSGTGRSGPPPSRGPTQEDYFPHGLASQVGPKGLIINGWAPQLLILSHSSIGGFLTHCGWNSTVEAIGLGVPLLAWPIRGDQNYNAKLVVAHLKLGFMISDNLSEKIKKHEIVKGIKTLMGDDDIKSRARNLAAIFQKGFPISSTTNLDVFRDMINNFT

SEQ ID NO: 37 - DNA, 1434 bp, Cannabis sativa
  source 1..1434: mol_type = genomic DNA
atgaagaggaccttgttgtttattccatctccaggtattggtcacctggtttctatgttggaatttgccaagagattgatccaatacgatgacaggttgttcatcaccatcttgtctatgaagttcccaaaccatgatgcctacatcaattctttggttccatccttgtctcagtccagagttaagttggctcatttgccacaagttgatcctccaccaccaaagttgttgaattctccagaatcttacatctacgtctacgtcgaatctttagttccacatgttagagatgctttgaagcacatagttccatctcactctaactctgaaactacccattctcaaggttgcttcgttatggttttggatttcttctgtatgccaatgatggatgttgctaacgaattgggtttgccatcttacatgttcatgccatctaacatcggtttcttgtcctctatgttgtacttggctactagacacgatcagatcagctctgaattgaaagaatctaacccagacgagtactccttgaagtcttttcataatccagttccatggtctgctttacctcaagcttatttctgtaaagacggtggttattctgcttgcgtaaaaatggctcaaagattcagagaaactaagggcatcatcgtcaattcctttgaaggtttggaagctcatggtgctacatcttttaatgatggtgaaactccaccaatctacatggttggtccagttgttaatttcaagggtcaaccacattcttccactgatcatgttcaaaacaacaggatcttcaagtggttggacgaacaaccacaatcctcagttgtttttttgtgctttgcttccttgggtactttcgatgcttcacaattgagagaaattgcctctggtttggaatgttctggtcatagatttttgtggtgcatcagagttcaacagccaaccattattgacgaaattttgccagaaggtttcttggaaagaatcggttctaaaggtatgatctgtaacgaatggactccacaagtagaagttttggctcataatgctgttggtggtttcgtttctcattgcggttggaattcaatcttggaatctttgtggtatggtgttccaatagttacttggccagtttacgctgaacaacaattgaatgctttccagatggttagggaattcgatttggctatcgaattgagattggactacagaaacagaggtcataaccagttggttaccgctgaagaaattggtaacgccatcaaaaaattgatggaaggtgatcacaacgtcgtcatgagaaaaaaaggtgcttctggtatctctagcatggtcttgtctagattctacacctgtaaagaaaacgtggtcttgagacaagatccaagatggtttgctattaaggctggtggtgaagaaaagtaa

SEQ ID NO: 38 - protein, 477 aa, Cannabis sativa
MKRTLLFIPSPGIGHLVSMLEFAKRLIQYDDRLFITILSMKFPNHDAYINSLVPSLSQSRVKLAHLPQVDPPPPKLLNSPESYIYVYVESLVPHVRDALKHIVPSHSNSETTHSQGCFVMVLDFFCMPMMDVANELGLPSYMFMPSNIGFLSSMLYLATRHDQISSELKESNPDEYSLKSFHNPVPWSALPQAYFCKDGGYSACVKMAQRFRETKGIIVNSFEGLEAHGATSFNDGETPPIYMVGPVVNFKGQPHSSTDHVQNNRIFKWLDEQPQSSVVFLCFASLGTFDASQLREIASGLECSGHRFLWCIRVQQPTIIDEILPEGFLERIGSKGMICNEWTPQVEVLAHNAVGGFVSHCGWNSILESLWYGVPIVTWPVYAEQQLNAFQMVREFDLAIELRLDYRNRGHNQLVTAEEIGNAIKKLMEGDHNVVMRKKGASGISSMVLSRFYTCKENVVLRQDPRWFAIKAGGEEK

SEQ ID NO: 39 - DNA, 1419 bp, Cannabis sativa
  source 1..1419: mol_type = genomic DNA
atggccagagtcgaattggtttttattccagctccagctattggtcatttggtttctactttggaattcgccaagagattgatccattacgatcataggttgttcatcaccgttttgtgcgaattctctttgaagtctcatttggatgcctacatcgattctttggttgcttctttgtctttggcccatagaatcaagttggttcatttgccattggttgattctccaccagttgagttgttgaagtccattgaaaatttcatctaccagtacatggaaagcttggtcccacatgttagaaaagctttgactgacatcgtgtcctctaactctaattactctcaaggtgatgttgtcttggtcttggattttttctgtatgccaatgatggatgtcgctaacgaattgggtttgccatcttacatgttcatgacttctaacttgggcctgttgtctttgatgttttacttggctaccagacacaaccagatctcttcagaattggaagaatctgatgctccattgagattgcaaggttttcaaaatccagttccatcctctgttttgccaactgctgctttctgtaaagatggtggttattctgcttacgttaagttggctcaaagattcagagaaactaagggcatcatcgtcaactcatttgaagaattggagtcctactccttctcctctatgaatgatggtgctgaaactccaccaatctatatggttggtccagttttggatttgaacggtcaaccacatccatctatggatcaagttcaaaacgacaagatcctgaagtggttggacgaacaacctcattcttctgttgttttcttgtgctttggctccatgggtaaatttggtgcttcacaattgagagaaattgcctccggtttacaaagatctggtcatagatttttgtggtccgttagagttcaacaacctactaccattgacgaaattttgccagaaggtttcttggaacaaatcggttctaaaggtatgatctgtaacgaatgggctccacaagttaagattttggctcattcagctgttggtggtttcttgtctcattgtggttggaactctatcttggaatctttgtggtatggtgttccagttgctacttggccaatctatgctgaacaacaattgaacgctttcaggatggttagagaatttggtttggctgttgatttgaggttggattacaaggatagaggtgatgatcatatcgtttccgccgaagaaattgaaactgctgtcaaacatttgatggaaggtgacaaagaggtccgtaaaaaggtcaaagaaatgtctgaaaccgccagaaagtctgttgaagaaggtggttcttctttcaccgctattggtaaattgatcaactccatcatcggctccaattactactga

SEQ ID NO: 40 - protein, 472 aa, Cannabis sativa
MARVELVFIPAPAIGHLVSTLEFAKRLIHYDHRLFITVLCEFSLKSHLDAYIDSLVASLSLAHRIKLVHLPLVDSPPVELLKSIENFIYQYMESLVPHVRKALTDIVSSNSNYSQGDVVLVLDFFCMPMMDVANELGLPSYMFMTSNLGLLSLMFYLATRHNQISSELEESDAPLRLQGFQNPVPSSVLPTAAFCKDGGYSAYVKLAQRFRETKGIIVNSFEELESYSFSSMNDGAETPPIYMVGPVLDLNGQPHPSMDQVQNDKILKWLDEQPHSSVVFLCFGSMGKFGASQLREIASGLQRSGHRFLWSVRVQQPTTIDEILPEGFLEQIGSKGMICNEWAPQVKILAHSAVGGFLSHCGWNSILESLWYGVPVATWPIYAEQQLNAFRMVREFGLAVDLRLDYKDRGDDHIVSAEEIETAVKHLMEGDKEVRKKVKEMSETARKSVEEGGSSFTAIGKLINSIIGSNYY

SEQ ID NO: 41 - DNA, 19 bp, synthetic construct
  misc_feature 1..19: note = Synthetic polynucleotide
  source 1..19: mol_type = other DNA
atggctttcgaaaaaaaca

SEQ ID NO: 42 - DNA, 21 bp, synthetic construct
  misc_feature 1..21: note = Synthetic polynucleotide
  source 1..21: mol_type = other DNA
tcaattattggactgtgctag

SEQ ID NO: 43 - DNA, 19 bp, synthetic construct
  misc_feature 1..19: note = Synthetic polynucleotide
  source 1..19: mol_type = other DNA
atgaacagagagcaaattc

SEQ ID NO: 44 - DNA, 21 bp, synthetic construct
  misc_feature 1..21: note = Synthetic polynucleotide
  source 1..21: mol_type = other DNA
ctactttctaccattcagctc

SEQ ID NO: 45 - DNA, 21 bp, synthetic construct
  misc_feature 1..21: note = Synthetic polynucleotide
  source 1..21: mol_type = other DNA
atgcggaatgtagagctcatc

SEQ ID NO: 46 - DNA, 22 bp, synthetic construct
  misc_feature 1..22: note = Synthetic polynucleotide
  source 1..22: mol_type = other DNA
ctagggcttaattcctatcacg

SEQ ID NO: 47 - DNA, 19 bp, synthetic construct
  misc_feature 1..19: note = Synthetic polynucleotide
  source 1..19: mol_type = other DNA
atggagacccaaacagaaa

SEQ ID NO: 48 - DNA, 25 bp, synthetic construct
  misc_feature 1..25: note = Synthetic polynucleotide
  source 1..25: mol_type = other DNA
ctaaaaggaagaaatataagcaact

SEQ ID NO: 49 - DNA, 18 bp, synthetic construct
  misc_feature 1..18: note = Synthetic polynucleotide
  source 1..18: mol_type = other DNA
atgcaggttttgggaatg

SEQ ID NO: 50 - DNA, 24 bp, synthetic construct
  misc_feature 1..24: note = Synthetic polynucleotide
  source 1..24: mol_type = other DNA
tcatagagtccttatgaagtgtac

SEQ ID NO: 51 - DNA, 24 bp, synthetic construct
  misc_feature 1..24: note = Synthetic polynucleotide
  source 1..24: mol_type = other DNA
atgggtgaagaagctatagttctg

SEQ ID NO: 52 - DNA, 18 bp, synthetic construct
  misc_feature 1..18: note = Synthetic polynucleotide
  source 1..18: mol_type = other DNA
tcactttgggctccacga

SEQ ID NO: 53 - DNA, 22 bp, synthetic construct
  misc_feature 1..22: note = Synthetic polynucleotide
  source 1..22: mol_type = other DNA
atggtcgacaagagagaagaag

SEQ ID NO: 54 - DNA, 22 bp, synthetic construct
  misc_feature 1..22: note = Synthetic polynucleotide
  source 1..22: mol_type = other DNA
tcatgatgatgatgatgatcct

SEQ ID NO: 55 - DNA, 18 bp, synthetic construct
  misc_feature 1..18: note = Synthetic polynucleotide
  source 1..18: mol_type = other DNA
atggagaagagtaatggc

SEQ ID NO: 56 - DNA, 25 bp, synthetic construct
  misc_feature 1..25: note = Synthetic polynucleotide
  source 1..25: mol_type = other DNA
ctagaaagatgatatataattaatc

SEQ ID NO: 57 - DNA, 21 bp, synthetic construct
  misc_feature 1..21: note = Synthetic polynucleotide
  source 1..21: mol_type = other DNA
atggaaaataaaacggagacc

SEQ ID NO: 58 - DNA, 25 bp, synthetic construct
  misc_feature 1..25: note = Synthetic polynucleotide
  source 1..25: mol_type = other DNA
ttacaacgatgaaatgtaagaaact

SEQ ID NO: 59 - DNA, 18 bp, synthetic construct
  misc_feature 1..18: note = Synthetic polynucleotide
  source 1..18: mol_type = other DNA
atgggatcccgttttgtt

SEQ ID NO: 60 - DNA, 21 bp, synthetic construct
  misc_feature 1..21: note = Synthetic polynucleotide
  source 1..21: mol_type = other DNA
ttacgtgttagggatctttcc

SEQ ID NO: 61 - DNA, 18 bp, synthetic construct
  misc_feature 1..18: note = Synthetic polynucleotide
  source 1..18: mol_type = other DNA
atgggtgtttttggatcg

SEQ ID NO: 62 - DNA, 24 bp, synthetic construct
  misc_feature 1..24: note = Synthetic polynucleotide
  source 1..24: mol_type = other DNA
tcatgacttcacaagttcaattaa

SEQ ID NO: 63 - DNA, 18 bp, synthetic construct
  misc_feature 1..18: note = Synthetic polynucleotide
  source 1..18: mol_type = other DNA
atggccgagccaaaaccg

SEQ ID NO: 64 - DNA, 26 bp, synthetic construct
  misc_feature 1..26: note = Synthetic polynucleotide
  source 1..26: mol_type = other DNA
ctacactcctgctataggattctcca

SEQ ID NO: 65 - DNA, 19 bp, synthetic construct
  misc_feature 1..19: note = Synthetic polynucleotide
  source 1..19: mol_type = other DNA
atggagactagagaaacaa

SEQ ID NO: 66 - DNA, 22 bp, synthetic construct
  misc_feature 1..22: note = Synthetic polynucleotide
  source 1..22: mol_type = other DNA
ttagaaagacaatatataagca

SEQ ID NO: 67 - DNA, 18 bp, synthetic construct
  misc_feature 1..18: note = Synthetic polynucleotide
  source 1..18: mol_type = other DNA
atggcagagattcgccag

SEQ ID NO: 68 - DNA, 22 bp, synthetic construct
  misc_feature 1..22: note = Synthetic polynucleotide
  source 1..22: mol_type = other DNA
tcattgttcgtcaatttgcatc

SEQ ID NO: 69 - DNA, 22 bp, synthetic construct
  misc_feature 1..22: note = Synthetic polynucleotide
  source 1..22: mol_type = other DNA
atggttcagccaagattcttga

SEQ ID NO: 70 - DNA, 21 bp, synthetic construct
  misc_feature 1..21: note = Synthetic polynucleotide
  source 1..21: mol_type = other DNA
tcattggtggatggacttcac

SEQ ID NO: 71 - DNA, 21 bp, synthetic construct
  misc_feature 1..21: note = Synthetic polynucleotide
  source 1..21: mol_type = other DNA
atgtccaagggtcataccatt

SEQ ID NO: 72 - DNA, 22 bp, synthetic construct
  misc_feature 1..22: note = Synthetic polynucleotide
  source 1..22: mol_type = other DNA
tcagtagtgatggattctttgc

SEQ ID NO: 73 - DNA, 22 bp, synthetic construct
  misc_feature 1..22: note = Synthetic polynucleotide
  source 1..22: mol_type = other DNA
atgtccaaagaaatctgggttg

SEQ ID NO: 74 - DNA, 24 bp, synthetic construct
  misc_feature 1..24: note = Synthetic polynucleotide
  source 1..24: mol_type = other DNA
ttacgtgaaattgttgatcatatc

SEQ ID NO: 75 - DNA, 24 bp, synthetic construct
  misc_feature 1..24: note = Synthetic polynucleotide
  source 1..24: mol_type = other DNA
atgaagaggaccttgttgtttatt

SEQ ID NO: 76 - DNA, 22 bp, synthetic construct
  misc_feature 1..22: note = Synthetic polynucleotide
  source 1..22: mol_type = other DNA
ttacttttcttccaccaccagc

SEQ ID NO: 77 - DNA, 19 bp, synthetic construct
  misc_feature 1..19: note = Synthetic polynucleotide
  source 1..19: mol_type = other DNA
atggccagagtcgaattgg

SEQ ID NO: 78 - DNA, 21 bp, synthetic construct
  misc_feature 1..21: note = Synthetic polynucleotide
  source 1..21: mol_type = other DNA
tcagtagtaattggagccgat

SEQ ID NO: 79 - DNA, 22 bp, synthetic construct
  misc_feature 1..22: note = Synthetic polynucleotide
  source 1..22: mol_type = other DNA
atggcctctgaaccatacaaat

SEQ ID NO: 80 - DNA, 22 bp, synthetic construct
  misc_feature 1..22: note = Synthetic polynucleotide
  source 1..22: mol_type = other DNA
tcaagagatgtgcaagtttctg

SEQ ID NO: 81 - DNA, 2163 bp, Cannabis sativa
  source 1..2163: mol_type = genomic DNA
atgggaaaaaattataagtcacttgacagtgtggttgctagtgatttcatagccttgggtatcacatctgaggtagcggagactcttcatgggcgtttagctgagattgtgtgtaattacggggcggccacccctcagacttggatcaatatagcaaaccacatcttatctcctgatctacccttttctctacaccaaatgctgttctatgggtgttacaaagatttcgggcccgcaccgcccgcatggataccggatccagagaaagtcaaatccaccaacttaggcgcgttgcttgaaaaaagaggcaaggaattcctgggcgtcaagtacaaggaccccatctcatctttctcccacttccaagagttctctgtacgtaatccggaggtttactggcgtaccgtccttatggatgagatgaagatatcattctccaaggacccggaatgtatactaagaagggatgacattaacaacccagggggcagtgagtggcttccgggtggataccttaatagtgcaaaaaactgtcttaacgtgaactcaaacaaaaagttaaacgatacaatgatcgtgtggcgtgatgagggcaatgatgaccttccgctaaataagttgacgcttgatcagctaagaaagcgtgtctggcttgtcggatatgccctagaggagatgggccttgagaagggatgcgcgatcgccatagatatgcctatgcatgtcgatgctgtggttatatatctggccatcgttctagcgggctatgttgttgtgagtattgccgatagcttcagtgcacccgaaatatcaacgagactgcgtttgtccaaagccaaagcaatattcacacaggatcatatcattagaggaaagaagcgtattccgctatattcaagggtggtggaggccaaaagcccgatggctatagtgataccctgttcaggcagcaacatcggagctgaattaagagatggagatatatcctgggactattttttagagagggcgaaagaatttaagaactgcgagttcaccgcaagggaacagccagtggatgcgtatacaaacatactattcagttcaggtactacgggagaaccaaaggctataccctggacgcaggcaaccccgctaaaagctgccgctgacgggtggtctcatttggatatcaggaagggtgatgttatcgtatggccaacaaatcttggatggatgatgggaccgtggcttgtgtacgctagcttgttaaatggagcttcaatcgctctatataatggttcccctcttgtcagcggttttgcgaagttcgtacaggatgccaaggtaactatgctgggagtcgttcctagcatcgtcaggagttggaaatcaactaactgtgtgtctggttacgactggtccactatcagatgttttagctccagtggtgaggcctccaacgtagatgagtacctttggcttatgggacgtgccaactataaaccggtaatagagatgtgtgggggtactgaaataggaggagcgttttcagctggcagttttcttcaggctcaaagcttgtctagtttctcttcacagtgtatgggctgtacgttatacattcttgataagaacggttatccaatgcccaaaaacaaaccaggtataggagaattggcgctgggtccagtcatgtttggcgctagtaaaactctactgaacggaaatcatcacgacgtttactttaaaggtatgcctactcttaatggagaagtacttaggcgtcatggcgatatctttgagttgacatccaatggctactatcacgcccatggcagggcggatgacaccatgaacatcggggggatcaaaatctccagtatagagatcgagagagtgtgtaacgaggtagacgaccgtgtattcgagacaacagccattggggttccacccctaggtggtggccccgaacaacttgttatcttttttgtgctgaaggactcaaatgacactaccattgatttaaatcaactacgtttgtcattcaatctgggattacaaaagaagttgaatcctttattcaaggtcacaagagtagtaccccttagttccctgccaagaactgcgacaaacaagataatgcgtagagtgctaaggcagcagtttagtcattttgagtaa

SEQ ID NO: 82 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 83 - DNA, 1158 bp, Cannabis sativa
  source 1..1158: mol_type = genomic DNA
atgaaccacttgagagcagaagggccggccagtgtactggctatagggacagccaaccccgaaaatatactgttgcaagatgagttcccagattattactttagagtgactaaatccgagcacatgacgcaacttaaggagaaattcaggaaaatatgcgacaaatctatgattagaaaaagaaattgtttcttaaatgaggagcatctaaagcagaatccccgtctggtagaacatgaaatgcaaactttggacgcgcgtcaagacatgctagttgtcgaagtgcctaaattaggcaaagacgcgtgtgcaaaggctataaaagagtggggccaaccgaagtccaaaattacacacctaatattcacttctgcgtccaccaccgacatgcccggagccgactaccactgtgcgaaacttctaggcctatccccttcagtcaagcgtgtaatgatgtatcaactggggtgctacggaggaggcaccgttttgaggattgcaaaggatatcgctgaaaataacaagggggctcgtgtacttgctgtgtgctgtgatatcatggcctgccttttcagaggcccctcagagtcagatcttgaactgttagtaggtcaggctatcttcggagatggcgctgcagccgtcatagttggggcggagcctgacgaatcagttggggagaggcccattttcgagctggtcagtacgggacagaccatcttgccaaatagcgagggcacgatcggaggccacataagggaggcgggtttgatatttgaccttcataaggatgtaccgatgttgatctccaataatattgagaagtgtcttattgaagcatttacccctattggtatttcagactggaacagtatcttctggattacgcatccgggaggtaaggcgattcttgataaagtcgaagaaaagctacacctgaagtcagacaagttcgttgactccagacacgttctttcagagcacggcaacatgagttcttccaccgtccttttcgtaatggacgagctgaggaaacgtagccttgaggaaggtaaaagtacgacaggagatgggtttgagtggggagtgttgtttggcttcggcccagggttaacagttgaacgtgtagtcgttagatctgtccctattaaatactaa

SEQ ID NO: 84 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 85 - DNA, 306 bp, Cannabis sativa
  source 1..306: mol_type = genomic DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaataa

SEQ ID NO: 86 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 87 - DNA, 1517 bp, Cannabis sativa
  source 1..1517: mol_type = genomic DNA
atcaataataatcttcatgggactctcattagtttgtaccttttcatttcaaactaattatcatactttattaaaccctcataataagaatcccaaaaactcattattatcttatcaacaccccaaaacaccaataattaaatcctcttatgataattttccctctaaatattgcttaaccaagaactttcatttacttggactcaattcacacaacagaataagctcacaatcaaggtccattagggcaggtagcgatcaaattgaaggttctcctcatcatgaatctgataattcaatagcaactaaaattttaaattttggacatacttgttggaaacttcaaagaccatatgtagtaaaagggatgatttcaatcgcttgtggtttgtttgggagagagttgttcaataacagacatttattcagttggggtttgatgtggaaggcattctttgctttggtgcctatattgtccttcaatttctttgcagcaatcatgaatcaaatttacgatgtggacatcgacaggataaacaagcctgatctaccactagtttcaggggaaatgtcaattgaaacagcttggattttgagcataattgtggcactaactgggttgatagtaactataaaattgaaatctgcaccactttttgttttcatttacatttttggtatatttgctgggtttgcctattctgttccaccaattagatggaagcaatatccttttaccaattttctaattaccatatcgagtcatgtgggcttagctttcacatcatattctgcaaccacatcagctcttggtttaccatttgtgtggaggcctgcttttagtttcatcatagcattcatgacagttatgggtatgactattgcttttgccaaagatatttcagatattgaaggcgacgccaaatatggggtatcaactgttgcaaccaaattaggtgctaggaacatgacatttgttgtttctggagttcttcttctaaactacttggtttctatatctattgggataatttggcctcaggttttcaagagtaacataatgatactttctcatgcaatcttagcattttgcttaatcttccagactcgtgagcttgctctagcaaattacgcctcggcgccaagcagacaattcttcgagtttatctggttgctatattatgctgaatactttgtatatgtatttatataagaccataatataacatatatatgtttattacataaaattgggacacaaaaacgtcaattatttggacaaaagtactcagaaagacctctttcactacaaggggaggccatttagttatacttgggtttcaatcaacaaatttataaatttttaagattttatttacaaaacattttcatgtgtaattaaatcgatcgtcatttattttttggatacaacttggttcaacttattttaattagagtgcttcgtaatttaactacaattatagaagggcattttataaaaatactggatttggggt

SEQ ID NO: 88 - protein, 398 aa, Cannabis sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 89 - DNA, 954 bp, synthetic construct
  misc_feature 1..954: note = Synthetic polynucleotide
  source 1..954: mol_type = other DNA
atgatcgaaggttcacctcatcatgaaagtgataacagcatagccacgaagattttgaatttcggccatacttgttggaagctacagaggccgtacgtcgttaaggggatgatttccattgcgtgcggtctgtttggcagggaattatttaacaacagacacttattcagttggggcctgatgtggaaggccttcttcgctcttgtacccattctgtccttcaacttttttgcagcgatcatgaatcaaatatacgatgtagacatcgatagaataaacaagcccgatttacctctggtatcaggcgaaatgagcatcgaaactgcgtggattttatcaatcatcgttgcattgactgggctgatagtgaccataaagttaaagtcagccccgttgtttgtcttcatatacatcttcggcattttcgcgggctttgcgtatagtgtacctcccattagatggaagcagtacccgtttactaactttcttattacaattagcagccatgtcggtcttgcattcacgtcctactcagccaccacatccgcactggggctaccgtttgtgtggcgtccagccttcagcttcatcatcgcattcatgacagtaatgggtatgacgatagcttttgcaaaggatataagtgatatcgagggtgacgctaagtatggagtgtctactgtggccacgaagctgggggcccgtaatatgactttcgtggtatcaggtgtactattgcttaattaccttgtttctatatcaatcggaattatttggccacaagttttcaaatccaatataatgatcctatcacacgctattttagcgttttgtttgatatttcagactagagagcttgcactagcgaattacgcgagtgccccgagtaggcagtttttcgagttcatatggctattatactatgctgagtactttgtttacgtatttatttaa

SEQ ID NO: 90 - protein, 317 aa, synthetic construct
  REGION 1..317: note = Synthetic polypeptide
MIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 91 - DNA, 1554 bp, Cannabis sativa
  source 1..1554: mol_type = genomic DNA
atgaacccccgtgaaaattttttgaaatgtttctctcaatacatacccaacaatgcaaccaacttaaagctggtatatactcaaaacaaccccctatatatgtctgttctaaatagtactatccataacttacgtttcacctcagataccacccctaaaccgctggtcatcgtgactccgtctcatgtttcacacatacagggcacgatattgtgctcaaaaaaggtcgggttacagattcgtacccgttcaggaggtcatgatagtgagggaatgtcttacatctcccaggtcccttttgtaattgtcgaccttcgtaatatgagatccataaagatcgacgttcattcacagacggcgtgggtagaggctggtgcaaccctaggtgaagtctactactgggtcaacgaaaaaaacgagaacttatcattagctgcggggtattgccctacagtttgtgccggaggtcattttggaggtggaggctacgggccactgatgaggaactacggtctggcagcagacaacattatagatgcacacctagtgaacgtgcatggtaaagttttagatagaaagtccatgggagaagatttgttttgggcactacgtggaggaggggctgagtcattcgggattattgtagcgtggaagatccgtctggtcgcagtccctaaatctacgatgttttccgtgaaaaagattatggaaattcacgagctagtgaaacttgtcaataagtggcagaatatagcatacaaatatgacaaggatctattgttgatgacgcatttcatcacaagaaacattacggacaatcaaggtaagaataagacggctattcacacttacttcagctccgtttttctaggaggggtagattccctagttgacctgatgaataagagttttcccgagttgggtattaaaaaaactgattgtagacagctgtcttggatcgacacaatcatattctactctggtgtggtaaactatgacaccgataatttcaacaaagaaatcttactggatagatcagccggtcaaaacggcgcgtttaaaatcaagctggattacgtaaagaagcctatacccgaatccgtatttgtacagattctggaaaagttatacgaggaagacattggggcgggtatgtacgctctttacccttacggcgggatcatggatgagatttccgaaagtgctatcccgttccctcatcgtgctggcattctgtacgagttatggtatatttgcagttgggagaagcaggaggataatgaaaagcacctaaattggattcgtaatatttataatttcatgactccctatgttagtaagaaccccagactggcctaccttaattatagagacctggacatcgggataaatgatccgaagaacccaaataactatacgcaggccaggatttggggggaaaagtatttcggaaagaactttgacagactggtgaaagttaagaccctggtggatccaaataattttttcaggaacgagcagagtattcccccgcttccacgtcacaggcattaa

SEQ ID NO: 92 - protein, 587 aa, Cannabis sativa
MNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRHMKCSTFSFWFVCKIIFFFFSFNIQTSIANPRENFLKCFSQYIPNNATNLKLVYTQNNPLYMSVLNSTIHN

SEQ ID NO: 93 - DNA, 1635 bp, synthetic construct
  misc_feature 1..1635: note = Synthetic polynucleotide
  source 1..1635: mol_type = other DNA
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcttccacggcatcgtcattaa

SEQ ID NO: 94 - protein, 544 aa, synthetic construct
  REGION 1..544: note = Synthetic polypeptide
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH